SEQUENCE LISTING (49 sequences)

SEQ ID NO: 1 - protein, 21 aa, Homo sapiens
MALPVTALLLPLALLLHAARP

SEQ ID NO: 2 - protein, 22 aa, Homo sapiens
MQSGTHWRVLGLCLLSVGVWGQ

SEQ ID NO: 3 - protein, 22 aa, Homo sapiens
MEQGKGLAVLILAIILLQGTLA

SEQ ID NO: 4 - protein, 21 aa, Homo sapiens
MEHSTFLSGLVLATLLSQVSP

SEQ ID NO: 5 - protein, 45 aa, Homo sapiens
TTTPAPRPPTPAPTIASQPLSLRPEACRPAAGGAVHTRGLDFACD

SEQ ID NO: 6 - protein, 24 aa, Homo sapiens
IYIWAPLAGTCGVLLLSLVITLYC

SEQ ID NO: 7 - protein, 122 aa, Artificial
  anti-BCMA VHH
QVQLVESGGGLVQPGGSLRLSCEASGFTLDYYAIGWFRQAPGKEREGVICISRSDGSTYYADSVKGRFTISRDNAKKTVYLQMISLKPEDTAAYYCAAGADCSGYLRDYEFRGQGTQVTVSS

SEQ ID NO: 8 - protein, 128 aa, Artificial
  anti-BCMA VHH
QVKLEESGGRLVQPRGSLRLSCAGSGRTFSTYGMAWFRQAPGKEREFVASKASMNYSGRTYYADSVKGRFTIARDNAKNMVFLQMNNLKPEDTAVYYCAAGTGCSTYGCFDAQIIDYWGKGTLVTVSS

SEQ ID NO: 9 - protein, 118 aa, Artificial
  anti-BCMA VHH
EVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSS

SEQ ID NO: 10 - protein, 120 aa, Artificial
  anti-BCMA VHH
AVQLVDSGGGLVQPGGSLRLSCVASGGIFVINAMGWYRQAPGKQRELVASIRGLGRTNYDDSVKGRFTISRDNANNTVYLQMNSLEPEDTAVYYCTVYVTLLGGVNRDYWGQGTQVTVSS

SEQ ID NO: 11 - protein, 119 aa, Artificial
  anti-BCMA VHH
EVQLVESGGGLVQAGGSLRLSCAASGRTFSSIVMGWFRQAPGKEREFVGAIMWNDGITYLQDSVKGRFTIFRDNAKNTVYLQMNSLKLEDTAVYYCAASKGRYSEYEYWGQGTQVTVSS

SEQ ID NO: 12 - protein, 128 aa, Artificial
  anti-BCMA VHH
EVQLVESGGGVVQAGGSLTVSCTASGFTFDRAVIVWFRQAPGKGREGVSFIKPSDGTIYYIDSLKGRFTISSDIAKNTVYLQMKSLESEDSAVYYCAASPEDWYTDWIDWSIYRWQHWGQGTQVTVSS

SEQ ID NO: 13 - protein, 119 aa, Artificial
  anti-BCMA VHH
EVQLVESGGGMVQAGDSLRLSCVQSTYTVNSDVMGWFRQAPGKEREFVGAIMWNDGITYLQDSVKGRFTIFRDNAKNTVYLQMNSLKLEDTAVYYCAASKGRYSEYEYWGQGTQVTVSS

SEQ ID NO: 14 - protein, 120 aa, Artificial
  anti-BCMA VHH
AVQLVESGGGLVQAGDSLRLSCTASGATLTNDHMAWFRQAPGKGREFVAAIDWSGRTTNYADPVEGRFTISRNNAKNTVYLEMNSLKLEDTAVYYCAVLRAWISYDNDYWGQGTQVTVSS

SEQ ID NO: 15 - protein, 120 aa, Artificial
  anti-BCMA VHH
QVQLVESGGGLVQAGGSLRLSCAASGGTLSKNTVAWFRQAPGKERGFVASITWDGRTTYYADSVKGRFTISRDNAKNTVYLQMNSLKPEDTAVYVCADLGKWPAGPADYWGQGTQVTVSS

SEQ ID NO: 16 - protein, 119 aa, Artificial
  anti-BCMA VHH
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSS

SEQ ID NO: 17 - protein, 119 aa, Artificial
  anti-BCMA VHH
AVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSS

SEQ ID NO: 18 - protein, 117 aa, Artificial
  anti-CD19 VHH
QVKLEESGGELVQPGGPLRLSCAASGNIFSINRMGWYRQAPGKQRAFVASITVRGITNYADSVKGRFTISVDKSKNTIYLQMNALKPEDTAVYYCNAVSSNRDPDYWGQGTQVTVSS

SEQ ID NO: 19 - protein, 123 aa, Artificial
  anti-CD19 VHH
QVKLEESGGGLVQAGESLRLSCAASGHTLSAYTMGWFRQAPEREREFVAAITRSGGRTSYGDSVKGRFTISRDTAKNTVYLQMNSLKPEDTAVYYCAADLRYRTVVNGLADYWGQGTQVTVSS

SEQ ID NO: 20 - protein, 119 aa, Artificial
  anti-CD19 VHH
QVKLEESGGGLVQAGGSLRLSCAASGRSFSNYDMGWFRQAPGKEREFVARISRRGDSTYYADSVKGRFIISRDNAKNTVYLQMNSLKPEDTAVYYCAARWRGSREIDYWGQGTQVTVSS

SEQ ID NO: 21 - protein, 130 aa, Artificial
  anti-CD33 VHH
QVQLAESGGGSVQAGGSLRLSCAASGYTYSSKRMGWFRQAPGKKREGVAGIVTEDGTTYYADSVKGRFTISQDNAKNTVYLQMNSLKPEDTATYYCAAGTTYGGLNDWWYTLHAGGYNYWGQGTQVTVSS

SEQ ID NO: 22 - protein, 126 aa, Artificial
  anti-CD33 VHH
EVQLAESGGGLVQAGGSLRLSCTASGFTFDDYVMGWFRQAPGKEREGVSCISWSGDTTYYADSVKGRFTASRDNAKNTLYLQMNSLKPEDTAMYYCAADQGKCSLASAEPDDMDYWGRGTLVTVSS

SEQ ID NO: 23 - protein, 121 aa, Artificial
  anti-CD33 VHH
EVQLAESGGGSVQAGGSLRLSCAASGVTFSSTSVAWFRQAPGKEREGVAYIYTGDSSTYYADSVKGRFTIAQDNAKNAVYLQMNSLKPEDTAMYYCAADGFLLNHRSYQYWGQGTQVTVSS

SEQ ID NO: 24 - protein, 127 aa, Artificial
  anti-CD33 VHH
EVQLVESGGGSVQAGGSLRLSCAASGYTYSINCMGWFRQAPGKEREGVAVISTGGGRTDYRDSVKGRFTISQDNAKNTVYLQMNSLKPEDTAMYYCAGKTTYPGYGCGLGRSAYNYWGQGTQVTVSS

SEQ ID NO: 25 - protein, 126 aa, Artificial
  anti-CD33 VHH
QIQLVESGGGSVQAGGSLRLSCVASGYIGGHYYMGWFRQAPGKEREGVAAIDIDSDGRTRYAGSVQGRFTISQDNAKNTLHLQMSSLKPEDTGMYYCAVGVGWVPARLTPQAVSYWGKGTLVTVSS

SEQ ID NO: 26 - protein, 128 aa, Artificial
  anti-CD33 VHH
EVQLVESGGGSVQAGGSLRLSCVASGYTYGINCMGWFRQAPGKEREGIAAISTGGGTTGVADSVKGRFTISQDNAKNTVYLQMNSLKPEDTAMYYCAARSTYSGYACVYSEVNGYNYRGQGTQVTVSS

SEQ ID NO: 27 - protein, 126 aa, Artificial
  anti-CD33 VHH
EVQLVESGGGSVQAGGSLRLSCVASGYTWCRYDMSWYRQAPGKEREFVSGFDNDGTTSYADSVKGRFTISQDNDKNTVYLQMNSLKPEDTAMYYCKEDRSRGSLDGRVCPLSYDNWGQGTQVTVSS

SEQ ID NO: 28 - protein, 124 aa, Artificial
  anti-CD33 VHH
EVQLVESGGGLVQAGGSLRLSCTASGFTFDDYVMGWFRQAPGKEREGVSCISWSGDTTYYADSVKGRFTVSRDNAKNTLYLQMNSLKPEDTAMYYCAADQSLCSLAPPYNYAYWGQGTQVTVSS

SEQ ID NO: 29 - protein, 126 aa, Artificial
  anti-CD33 VHH
QVQLVESGGGLVQAGGSLRLSCTASGFTFDNYVMGWFRQAPGKEREGVSCIGWSGGSTYYADSVKGRFTISRDNAKNTLYLQMNSLKPEDTAMYYCAADQGKCSLGSAGADDMDYWGRGTLVTVSS

SEQ ID NO: 30 - protein, 120 aa, Artificial
  anti-CD33 VHH
EVQLVESGGGSVQAGGSLRLSCAAPGYTYCTYDMMWYRQALGKEREFVSAVYTDGSTLYADSVKGRFTISQDNAKNTLYLQMNSLKPEDTAMYYCKTETAVTYDKPCDFWGQGTQVTVSS

SEQ ID NO: 31 - protein, 124 aa, Artificial
  anti-CD33 VHH
QVQLVESGGGLVQPGGSLRLSCAASGSIFSITAMGWYRQAPGKQRELVATVTSGGSTKYVESVKGRFTISNDNAKNTVYLQMNSLKPEDTAVYYCWADIVARWNSDYNVYDDYWGQGTQVTVSS

SEQ ID NO: 32 - protein, 121 aa, Artificial
  anti-CD33 VHH
QVKLEESGGGLVQEGGSLRLSCAASGTIYTDSTIYWYRQAPGKQRELVASITRGGDTKYVDSVKGRFAISSDNAKNTVYLQMNSLKPEDTAVYYCNLYYRTGPSSYYRNDWGQGTQVTVSS

SEQ ID NO: 33 - protein, 112 aa, Artificial
  anti-CD33 VHH
AVQLVESGGGLVQPGGSLRLSCAVSGIYYSINLMGWYRQAPGKPRELAASITDSGITNYADSVKGRFTISRDNAKNTVHLQMNNLKPEDTAVYYCKPMGVNWGQGIQVTVSS

SEQ ID NO: 34 - protein, 121 aa, Artificial
  anti-CD33 VHH
AVQLVESGGGLVQAGGSLRLSCTASGSILRTYEMGWYRQAPGSQRELVASITSDGDTNYVDSVKGRFTISRDNGKNMVYLQMNSLKPDDTAVYYCNAVILSPSYSMRTSHWGQGTQVTVSS

SEQ ID NO: 35 - protein, 124 aa, Artificial
  anti-CD33 VHH
QVQLVESGGGLVQAGGSLRLSCAASGNVFRFNIMGWYRQAPGNQRELVASIDDGGDRSYADSVEGRFTISRENGKKIMYLQMNSLKPEDTAVYYCAAGLGTYLNGRVSMATNYWGQGTQVTVSS

SEQ ID NO: 36 - protein, 121 aa, Artificial
  anti-CD33 VHH
EVQLVESGGGLVQAGGSLRLSCAASGDTFSFAAMGWYRQAPGKQRELVASISFSGGESTVYANSVRGRFTISGDNAKNRVSLEMNNLKPEDTAVYYCTAKRGPYLPGPEYWGQGTQVTVSS

SEQ ID NO: 37 - protein, 121 aa, Artificial
  anti-CLL-1 VHH
QVQLVESGGDLVRPGGSLRLSCAASGFTFSIYDMNWVRQAPGKGLEWVAGISGNGYSTSYAESVKGRFTISRDNAKNTVYLQLSSLKFEDTAMYYCVRDAERWDENDLRRKGQGTQVTVSS

SEQ ID NO: 38 - protein, 121 aa, Artificial
  anti-CLL-1 VHH
EVQLVESGGGSVQAGGSLRLSCAASGVTYSSACMGWFRQAPGKGREVVAVLYAGGSTTHYASSVKERFTISQDNAKNTVYLQMNSLKPEDTAVYYCAAALGDRSSCEWRYWGQGTQVTVSS

SEQ ID NO: 39 - protein, 112 aa, Artificial
  anti-CLL-1 VHH
QVQLVESGGGLVQPGGSLRLSCAASGFTFSVYDMNWFRQAPGKGLEWVSGITGNGYTTSYADSVKGRFTISRDNAKNTLYLQLNSLKSEDTAMYYCAKETNRGQGTQVTVSS

SEQ ID NO: 40 - protein, 126 aa, Artificial
  anti-CLL-1 VHH
QVQLAESGGGLVQPGGSLRLSCVASGFTFSSYDMSWVRQAPGKGVEWVSTINSGGGSTYYAESAKGRFTISRDNAKNTLYLQLNSLKTEDTAMYYCVKGFPDDDGPGELSREYNYWGQGTQVTVSS

SEQ ID NO: 41 - protein, 114 aa, Artificial
  anti-CLL-1 VHH
EVQLVESGGALVQPGGSLRLSCTASGFLFRVYDMNWVRQAPGKGVEWIVGITNNGYTTAYADSVKGRFTISRDNTENTLFLQMNSLKPEDTAMYYCQTDNGRVRGQGTQVTVSS

SEQ ID NO: 42 - protein, 116 aa, Artificial
  anti-CLL-1 VHH
QVQLVESGGGSVQAGGALSLSCAASGYTVRIDYMGWYRQTPGKGREPVATIASNGGTAYADSVEGRFTISQDNAKNSVYLQMNTLKPGDTAMYYCAAGTWPTLTYFGQGTQVTVSS

SEQ ID NO: 43 - protein, 136 aa, Artificial
  anti-CLL-1 VHH
QVQLAESGGGLVQTGGSLRLSCTASGLNFGLYAMGWFRQAPGKEREGVSCINGGGGITVYSDFVKSRFTISRDNAKNTLYLQMNSLKPDDTATYYCAADRSPFGSCSSDWSRSSDWSRMAEKFGYWGQGTQVTVSS

SEQ ID NO: 44 - protein, 119 aa, Artificial
  anti-CLL-1 VHH
QVQLVESGGGSVQAGGSLRLSCVVSAATNCRYIAWYRQAPGKAREFVSTLGSDGNTNYADSVKGRFTISQGNIKNMAYLEMNSLKPEDTGMYYCGTRCQIGDDWRSSDWAQGTQVTVSS

SEQ ID NO: 45 - protein, 123 aa, Artificial
  anti-CLL-1 VHH
QVHLVESGGGSVQSGGSLRLSCAASGYAYRSYCMGWFRQAPGKVLEGVAAIESDGTTTYADSVMGRFTISQDNAKNALYLQMNSLKPEDTAMYHCAAVKGSCDSASSDTPSYWGQGTQVTVSS

SEQ ID NO: 46 - protein, 121 aa, Artificial
  anti-CLL-1 VHH
EVQLVESGGDLVRPGGSLRLSCAASGFTFSIYDMNWVRQAPGKGLEWVAGISGNGYSTSYAESVKGRFTISKDNAKNTVYLQLSSLKFEDTAMYYCVRGGEKWDENDLRRKGQGTQVTVSS

SEQ ID NO: 47 - protein, 126 aa, Artificial
  anti-CLL-1 VHH
QVRLVESGGGSVQSGGSLRLSCAASGYARSSTCLGWFRQAPGKEVEGVAIIGRDGSTGYADSVKGRFTISQDNAKNTLYLHMDSLKPEDTAMYYCAAVEGGCEVSEGTGEQQLAYWGQGTQVTVSS

SEQ ID NO: 48 - protein, 122 aa, Artificial
  anti-CLL-1 VHH
QVHLMESGGGLVQPGESLRLSCAASGFIFANYEMSWVRQAPGKVLEWVSGINSRGNATYYADSVKGRFTISRDNAEHTLYLQMNSLKPEDTAMYHCVVGGMTTDQGSPDFYWGQGTQVTVSS

SEQ ID NO: 49 - protein, 122 aa, Artificial
  anti-CLL-1 VHH
QVKLVESGGGLVQPGGSLRLSCVASGFAFSSADMSWVRQAPGKGVEAVSVINRDGASTYYADSVKGRFTISRDNAKSTLYLQMNSLKPEDTAMYHCAVVPENEYESGSYNYWGQGTQVTVSS

(19) United States
(12) Patent Application Publication
(10) Pub. No.: US 2023/0144237 A1
(43) Pub. Date: May 11, 2023

(54) ENGINEERED CELLS AND USES THEREOF

(71) Applicant: Nanjing Legend Biotech Co., Ltd., Nanjing, Jiangsu (CN)

(72) Inventors: Zhongyuan Tu, Nanjing (CN); Hezhong Zhang, Nanjing (CN); Yafeng Zhang, Nanjing (CN); Shu Wu, Nanjing (CN)

(21) Appl. No.: 17/605,290

(22) PCT Filed: Apr. 22, 2020
(86) PCT No.: PCT/CN2020/086073

§ 371 Date: Oct. 21, 2021

(30) Foreign Application Priority Data
Apr. 22, 2019 (CN) PCT/CN2019/083748

(51) Int. Cl. C07K 14/725; C12N 5/0783; C12N 15/86; C07K 16/30; C07K 16/28; A61K 35/17

(52) U.S. Cl. CPC C07K 14/7051; C12N 5/0636; C12N 15/86; C07K 16/30; C07K 16/2866; C07K 16/28; A61K 35/17; C12N 2740/15043; C12N 2501/2302; C07K 2319/00

(57) ABSTRACT

The present disclosure provides systems for inducing activity of immune cells and methods of immunotherapy. Systems of the present disclosure for inducing immune cell activity comprise a chimeric antigen receptor, a T cell receptor, and various combinations thereof.

### CROSS REFERENCE TO RELATED APPLICATIONS

This application is a National Stage Application under 35 U.S.C. § 371 and claims the benefit of International Application No. PCT/CN2020/086073, filed on Apr. 22, 2020, which claims the benefit of International Application No. PCT/CN2019/083748, filed on Apr. 22, 2019. The entire contents of the foregoing applications are incorporated herein by reference.

### SEQUENCE LISTING

This application contains a Sequence Listing that has been submitted electronically as an ASCII text file named 51624-0009US1SEQ.txt. The ASCII text file, created on Jun. 16, 2022, is 49.9 kilobytes in size. The material in the ASCII text file is hereby incorporated by reference in its entirety.

### BACKGROUND

Effector cell activities often involve binding of a ligand to a receptor bound on a membrane of the effector cell. Such interaction between the ligand and the membrane-bound receptor, often via its extracellular binding domain, may result in a conformational and/or chemical modification to the receptor itself, which in turn can yield an array of intracellular signaling that cumulates in effector cell activation. Attempts to harness this interaction for the development of immune cell therapies against cancer cells have shown promising efficacy against hematologic malignancies. However, there are a number of drawbacks including off-target toxicity and undesired cytokine release syndrome in treated subjects. This and other side effects can further exuberate into inflammatory responses, organ failure, and in extreme cases, death.

### SUMMARY

In view of the foregoing, there exists a considerable need for alternative compositions and methods to carry out immunotherapy. The compositions and methods of the present disclosure address this need, and provide additional advantages as well. The various aspects of the disclosure provide systems, compositions, and methods for inducing activity of immune cells.

In one aspect, the present disclosure provides a system for inducing activity of an immune cell, comprising: (a) a chimeric antigen receptor (CAR) comprising a first antigen binding domain which exhibits specific binding to a first epitope, a transmembrane domain, and an intracellular signaling domain devoid of a signaling domain of CD3 zeta (ζ); and (b) a modified T cell receptor (TCR) complex comprising a second antigen binding domain that exhibits specific binding to a second epitope, wherein said second antigen binding domain is linked to: (i) at least one TCR chain selected from an alpha chain, a beta chain, a gamma chain and a delta chain of a T cell receptor, (ii) an epsilon chain, a delta chain, and/or a gamma chain of cluster of differentiation 3 (CD3), or (iii) a CD3 zeta chain.

In some embodiments, binding of the first antigen binding domain to the first epitope, and/or binding of the second antigen binding domain to the second epitope activates an immune cell activity of an immune cell expressing the system.

In some embodiments, two or more antigen binding domains are linked to, optionally in tandem, (i) at least one TCR chain selected from an alpha chain, a beta chain, a gamma chain and a delta chain of a T cell receptor, (ii) an epsilon chain, a delta chain, and/or a gamma chain of cluster of differentiation 3 (CD3), (iii) a CD3 zeta chain, and wherein binding of the two or more antigen binding domains to their respective epitopes activates an immune cell activity of an immune cell expressing the system.

In some embodiments, said immune cell activity is selected from the group consisting of: clonal expansion of the immune cell; cytokine release by the immune cell; cytotoxicity of the immune cell; proliferation of the immune cell; differentiation, dedifferentiation or transdifferentiation of the immune cell; movement and/or trafficking of the immune cell; exhaustion and/or reactivation of the immune cell; and release of other intercellular molecules, metabolites, chemical compounds, or combinations thereof by the immune cell.

In some embodiments, the first epitope and the second epitope are the same. In some embodiments, the first epitope and the second epitope are different.

In some embodiments, the first antigen binding domain and the second antigen binding domain comprise the same amino acid sequence. In some embodiments, the first antigen binding domain and the second antigen binding domain comprise different amino acid sequence.

In some embodiments, the second antigen binding domain comprises a heterologous sequence exhibiting binding to the second epitope.

In some embodiments, the modified TCR comprises a third antigen binding domain linked to: (i) said second antigen binding domain, (ii) at least one TCR chain selected from the alpha chain, the beta chain, the gamma chain and the delta chain of a T cell receptor, (iii) the epsilon chain, the delta chain, and/or the gamma chain of cluster of differentiation 3 (CD3), or (iv) the CD3 zeta chain.

In some embodiments, said intracellular signaling domain of said CAR is devoid of an immunoreceptor tyrosine-based activation motif (ITAM).

In some embodiments, said CAR further comprises a co-stimulatory domain. In some embodiments, said co-stimulatory domain comprises a signaling domain of a MHC class I molecule, a TNF receptor protein, an immunoglobulin-like protein, a cytokine receptor, an integrin, a signaling lymphocytic activation molecule (SLAM protein), an activating NK cell receptor, or a Toll ligand receptor.

In some embodiments, said co-stimulatory domain comprises a signaling domain of a molecule selected from: 2B4/CD244/SLAMF4, 4-1BB/TNFSF9/CD137, B7-1/CD80, B7-2/CD86, B7-H1/PD-L1, B7-H2, B7-H3, B7-H4, B7-H6, B7-H7, BAFF-R/TNFRSF13C, BAFF/BLyS/TNFSF13B, BLAME/SLAMF8, BTLA/CD272, CD100 (SEMA4D), CD103, CD11a, CD11b, CD11c, CD11d, CD150, CD160 (BY55), CD18, CD19, CD2, CD200, CD229/SLAMF3, CD27 Ligand/TNFSF7, CD27/TNFRSF7, CD28, CD29, CD2F-10/SLAMF9, CD30 Ligand/TNFSF8, CD30/TNFRSF8, CD300a/LMIR1, CD4, CD40 Ligand/TNFSF5, CD40/TNFRSF5, CD48/SLAMF2, CD49a, CD49D, CD49f, CD53, CD58/LFA-3, CD69, CD7, CD8 α, CD8 β, CD82/Kai-1, CD84/SLAMF5, CD90/Thy1, CD96, CDS, CEACAM1, CRACC/SLAMF7, CRTAM, CTLA-4, DAP12, Dectin-1/CLEC7A, DNAM1 (CD226), DPPIV/CD26, DR3/TNFRSF25, EphB6, GADS, Gi24/VISTA/B7-H5, GITR Ligand/TNFSF18, GITR/TNFRSF18, HLA Class I, HLA-DR, HVEM/TNFRSF14, IA4, ICAM-1, ICOS/CD278, Ikaros, IL2R β, IL2R γ, IL7R α, Integrin α4/CD49d, Integrin α4β1, Integrin α4β7/LPAM-1, IPO-3, ITGA4, ITGA6, ITGAD, ITGAE, ITGAL, ITGAM, ITGAX, ITGB1, ITGB2, ITGB7, KIRDS2, LAG-3, LAT, LIGHT/TNFSF14, LTBR, Ly108, Ly9 (CD229), lymphocyte function associated antigen-1 (LFA-1), Lymphotoxin-α/TNF-β, NKG2C, NKG2D, NKp30, NKp44, NKp46, NKp80 (KLRF1), NTB-A/SLAMF6, OX40 Ligand/TNFSF4, OX40/TNFRSF4, PAG/Cbp, PD-1, PDCD6, PD-L2/B7-DC, PSGL1, RELT/TNFRSF19L, SELPLG (CD162), SLAM (SLAMF1), SLAM/CD150, SLAMF4 (CD244), SLAMF6 (NTB-A), SLAMF7, SLP-76, TACI/TNFRSF13B, TCL1A, TCL1B, TIM-1/KIM-1/HAVCR, TIM-4, TL1A/TNFSF15, TNF RII/TNFRSF1B, TNF-α, TRANCE/RANKL, TSLP, TSLP R, VLA1, and VLA-6.

In some embodiments, said first antigen binding domain and/or said second antigen binding domain comprises a Fab, a Fab′, a F(ab′)₂, an Fv, a single-chain Fv (scFv), minibody, a diabody, a single-domain antibody, a light chain variable domain (VL), or a variable domain (V_{H}H) of camelid antibody.

In some embodiments, said first antigen binding domain and/or said second antigen binding domain comprises a receptor. In some embodiments, said first antigen binding domain and/or said second antigen binding domain comprises a ligand for a receptor. In some embodiments, said first epitope and said second epitope are present on different antigens. In some embodiments, said first epitope and said second epitope are present on a common antigen. In some embodiments, said first epitope and/or said second epitope are present on one or more cell surface antigens. In some embodiments, said one or more cell surface antigens are tumor associated antigens, tyrosine kinase receptors, serine kinase receptors, and G-protein coupled receptors. In some embodiments, said first epitope and/or said second epitope is present on a universal antigen. In some embodiments, said first epitope and/or said second epitope is present on a neoantigen. In some embodiments, said first epitope and/or said second epitope is a neoepitope.

In some embodiments, said first epitope and/or said second epitope is present on a tumor-associated antigen. In some embodiments, the tumor-associated antigen is selected from the group consisting of: 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, BCMA, b-Catenin, bcr-abl, bcr-abl p190 (e1a2), bcr-abl p210 (b2a2), bcr-abl p210 (b3a2), BING-4, CA-125, CAG-3, CAIX, CAMEL, Caspase-8, CD171, CD19, CD20, CD22, CD23, CD24, CD30, CD33, CD38, CD44v7/8, CD70, CD123, CD133, CDC27, CDK-4, CEA, CLCA2, CLL-1, CTAG1B, Cyp-B, DAM-10, DAM-6, DEK-CAN, DLL3, EGFR, EGFRvIII, EGP-2, EGP-40, ELF2, Ep-CAM, EphA2, EphA3, erb-B2, erb-B3, erb-B4, ES-ESO-1a, ETV6/AML, FAP, FBP, fetal acetylcholine receptor, FGF-5, FN, FR-α, G250, GAGE-1, GAGE-2, GAGE-3, GAGE-4, GAGE-5, GAGE-6, GAGE-7B, GAGE-8, GD2, GD3, GnT-V, Gp100, gp75, GPC3, GPC-2, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, L1-CAM, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, and κ-light chain.

In some embodiments, said first epitope and/or said second epitope is present on an immune checkpoint receptor or immune checkpoint receptor ligand. In some embodiments, said immune checkpoint receptor or immune checkpoint receptor ligand is PD-1, PD-L1, PD-L2, CTLA-4, TIM-3, TIGIT, LAG3, BLTA, CD47 or CD40.

In some embodiments, said first epitope and/or said second epitope is present on a cytokine or a cytokine receptor. In some embodiments, said cytokine or cytokine receptor is CCR2b, CXCR2 (CXCL1 receptor), CCR4 (CCL17 receptor), Gro-a, IL-2, IL-7, IL-15, IL-21, IL-12, Heparanase, CD137L, LEM, Bcl-2, CCL17, CCL19 or CCL2.

In some embodiments, said first epitope and/or said second epitope is present on an antigen presented by a major histocompatibility complex (MHC). In some embodiments, the MHC is HLA class 1. In some embodiments, the MHC is HLA class 2.

In another aspect, the present disclosure provides an isolated host cell expressing the above system of the present disclosure. In some embodiments, the host cell is an immune cell. In some embodiments, the immune cell is a lymphocyte. In some embodiments, the lymphocyte is a T cell. In some embodiments, the T cell is a CD8+ T cell. In some embodiments, the T cell is a CD4+ T cell. In some embodiments, the lymphocyte is γδ T cell. In some embodiments, the γδ T cell is Vγ9δ2 T cell. In some embodiments, the γδ T cell is Vδ1 T cell. In some embodiments, the lymphocyte is the γδ T cell comprising Vγ9δ2 TCR, Vγ10/Vδ2 TCR, and/or Vγ2/Vδ2 TCR. In some embodiments, the lymphocyte is the γδ T cell comprising Vγ9δ2 TCR. In some embodiments, the lymphocyte is the γδ T cell comprising Vγ10/Vδ2 TCR. In some embodiments, the lymphocyte is the γδ T cell comprising Vγ2/Vδ2 TCR. In some embodiments, the lymphocyte is a natural killer (NK) cell, a KHYG such as KHYG-1 cell or a derivative thereof. In some embodiments, the host cell exhibits specific binding to two antigens simultaneously present in a target cell.

In another aspect, the present disclosure provides an antigen-specific immune cell comprising above system of the present disclosure. In some embodiments, the antigen binding domain linked to the CAR primarily mediates interaction between the immune cell and a target cell, and the antigen binding domain linked to the TCR complex primarily mediates an immune cell activity when the interaction between the immune cell and the target cell takes place.

In some embodiments, said immune cell activity is selected from the group consisting of: clonal expansion of the immune cell; cytokine release by the immune cell; cytotoxicity of the immune cell; proliferation of the immune cell; differentiation, dedifferentiation or transdifferentiation of the immune cell; movement and/or trafficking of the immune cell; exhaustion and/or reactivation of the immune cell; and release of other intercellular molecules, metabolites, chemical compounds, or combinations thereof by the immune cell. In some embodiments, said immune cell is a lymphocyte. In some embodiments, said lymphocyte is a T cell. In some embodiments, said T cell is a CD4+ T cell or a CD8+ T cell. In some embodiments, the lymphocyte is γδ T cell. In some embodiments, the γδ T cell is Vγ9δ2 T cell. In some embodiments, the γδ T cell is Vδ1 T cell. In some embodiments, the lymphocyte is the γδ T cell comprising Vγ9δ2 TCR, Vγ10/Vδ2 TCR, and/or Vγ2/Vδ2 TCR. In some embodiments, the lymphocyte is the γδ T cell comprising Vγ9δ2 TCR. In some embodiments, the lymphocyte is the γδ T cell comprising Vγ10/Vδ2 TCR. In some embodiments, the lymphocyte is the γδ T cell comprising Vγ2/Vδ2 TCR.

In some embodiments, the modified TCR complex comprises two or more antigen binding domains comprising heterologous sequences that are linked to, optionally in tandem, (i) at least one TCR chain selected from an alpha chain, a beta chain, a gamma chain and a delta chain of a T cell receptor, (ii) an epsilon chain, a delta chain, and/or a gamma chain of cluster of differentiation 3 (CD3), (iii) a CD3 zeta chain. In some embodiments, said lymphocyte is a natural killer (NK) cell, a KHYG cell such as KHYG-1 cell or a derivative thereof.

In another aspect, the present disclosure provides a population of immune cells, individual immune cells expressing above system of the present disclosure. In some embodiments, said population of immune cells comprises at most about 10¹¹ cells. In some embodiments, said immune cells comprise lymphocytes. In some embodiments, the lymphocytes are T cells. In some embodiments, the T cells are CD4+ T cells. In some embodiments, the T cells are CD8+ T cells. In some embodiments, the lymphocyte is γδ T cell. In some embodiments, the γδ T cell is Vγ9δ2 T cell. In some embodiments, the γδ T cell is Vδ1 T cell. In some embodiments, the lymphocytes are natural killer (NK) cells, a KHYG cell such as KHYG-1 cell or a derivative thereof.

In another aspect, the present disclosure provides a method of inducing activity of an immune cell, comprising: (a) expressing above system of the present disclosure in an immune cell; and (b) contacting a target cell with the immune cell under conditions that induce said activity of the immune cell and/or the target cell. In some embodiments, binding of the first antigen binding domain to the first epitope and/or binding of the second antigen binding domain to the second epitope activates cytotoxicity of the immune cell.

In some embodiments, two or more antigen binding domains comprise heterologous sequences that are linked to, optionally in tandem, (i) at least one TCR chain selected from an alpha chain, a beta chain, a gamma chain and a delta chain of a T cell receptor, (ii) an epsilon chain, a delta chain, and/or a gamma chain of cluster of differentiation 3 (CD3), (iii) a CD3 zeta chain.

In some embodiments, the target cell is a cancer cell. In some embodiments, the target cell is a hematopoietic cell. In some embodiments, the target cell is a solid tumor cell. In some embodiments, the target cell is a cell identified in one or more of heart, blood vessels, salivary gland, esophagus, stomach, liver, gallbladder, pancreas, intestine, colon, rectum, anus, endocrine gland, adrenal gland, kidney, ureter, bladder, lymph node, tonsils, adenoid, thymus, spleen, skin, muscle, brain, spinal cord, nerve, ovary, fallopian tube, uterus, vagina, mammary gland, testes, prostate, penis, pharynx, larynx, trachea, bronchi, lung, diaphragm, cartilage, ligaments, and tendon.

In some embodiments, said immune cell is a lymphocyte. In some embodiments, the lymphocyte is a T cell. In some embodiments, the T cell is a CD4+ T cell or CD8+ T cell. In some embodiments, the lymphocyte is γδ T cell. In some embodiments, the γδ T cell is Vγ9δ2 T cell. In some embodiments, the γδ T cell is Vδ1 T cell. In some embodiments, the lymphocyte is the γδ T cell comprising Vγ9δ2 TCR, Vγ10/Vδ2 TCR, and/or Vγ2/Vδ2 TCR. In some embodiments, the lymphocyte is the γδ T cell comprising Vγ9δ2 TCR. In some embodiments, the lymphocyte is the γδ T cell comprising Vγ10/Vδ2 TCR. In some embodiments, the lymphocyte is the γδ T cell comprising Vγ2/Vδ2 TCR.

In some embodiments, binding of the two or more antigen binding domains to their respective epitopes activates cytotoxicity of an immune cell expressing the system and increases persistence of said cytotoxicity, as compared to binding of the first antigen binding domain to the first epitope alone, when said system is expressed in an immune cell in a subject. In some embodiments, the lymphocyte is a natural killer (NK) cell, a KHYG cell such as KHYG-1 cell or a derivative thereof.

In another aspect, the present disclosure provides a composition comprising one or more polynucleotides that encodes: (a) a chimeric antigen receptor (CAR) comprising a first antigen binding domain having binding specificity for a first epitope, a transmembrane domain, and an intracellular signaling domain that is devoid of signaling domain of CD3 zeta; and (b) a modified T cell receptor (TCR) complex comprising a second antigen binding domain which exhibits specific binding to a second epitope, wherein said second antigen binding domain is linked to: (i) at least one TCR chain selected from an alpha chain, a beta chain, a gamma chain and a delta chain of a T cell receptor, (ii) an epsilon chain, a delta chain, and/or a gamma chain of cluster of differentiation 3 (CD3), or (iii) a CD3 zeta chain.

In some embodiments, the second antigen binding domain comprises a heterologous sequence exhibiting binding to the second epitope. In some embodiments, the one or more polynucleotides comprise a promoter operably linked thereto.

In another aspect, the present disclosure provides a method of producing a modified immune cell, comprising genetically modifying the immune cell by expressing the above composition of the present disclosure in said immune cell, thereby producing said modified immune cell.

In another aspect, the present disclosure provides a method of treating a cancer of a subject, said subject comprising a target cell expressing one or more antigens, the method comprising: (a) administering to the subject an antigen-specific immune cell comprising above system of the present disclosure, wherein the expressed one or more antigens are recognized by the first and/or second antigen binding domain, and (b) contacting the target cell with the antigen-specific immune cell via the first and/or second antigen binding domains under conditions that induces an immune cell activity of the immune cell against the target cell, thereby inducing death of the target cell of the cancer.

In some embodiments, the method further comprising genetically modifying an immune cell to yield said antigen-specific immune cell. In some embodiments, said cancer is selected from: bladder cancer, bone cancer, brain cancer, breast cancer, cervical cancer, colon cancer, esophageal cancer, gastric cancer, glioma, head and neck cancer, kidney cancer, leukemia, acute myeloid leukemia (AML), multiple myeloma, liver cancer, lung cancer, lymphoma, melanoma, mesothelioma, medulloblastoma, ovarian cancer, pancreatic cancer, prostate cancer, rectal cancer, skin cancer, testicular cancer, tracheal cancer, and vulvar cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic of a CAR-TCR-T system comprising antigen-binding domains, as shown in the black and white striped oval and black oval, capable of binding an antigen, for example a tumor-associated antigen, wherein the CAR of the CAR-TCR-T system comprises an intracellular signaling domain devoid of CD3-zeta signaling chain.

FIG. 2A shows a modified TCR complex comprising an antigen binding domain fused to an epsilon chain of CD3. FIG. 2B shows a modified TCR complex comprising an antigen binding domain fused to a delta chain of CD3. FIG. 2C shows a modified TCR complex comprising an antigen binding domain fused to a gamma chain of CD3. FIG. 2D shows a modified TCR complex comprising an antigen binding domain fused to an alpha chain of TCR, or a gamma chain of TCR.

FIG. 2E shows a modified TCR complex comprising an antigen binding domain fused to a beta chain of TCR, or a delta chain of TCR. FIG. 2F shows a modified TCR complex comprising two antigen binding domains. A first antigen binding domain is fused to a first epsilon chain and a second antigen binding domain is fused to a second epsilon chain of CD3. FIG. 2G shows a modified TCR complex comprising two antigen binding domains. A first antigen binding domain is fused to an epsilon chain and a second antigen binding domain is fused to a gamma chain of CD3. FIG. 2H shows a modified TCR complex comprising a first antigen binding domain fused to a second antigen binding domain, which in turn in fused to an epsilon chain of CD3. FIG. 2I shows a modified TCR complex comprising two antigen binding domains. A first antigen binding domain is fused to an alpha chain of TCR and a second antigen binding domain is fused to a beta chain of TCR, or a first antigen binding domain is fused to a gamma chain of TCR and a second antigen binding domain is fused to a delta chain of TCR. FIG. 2J shows a modified TCR complex comprising two identical antigen binding domains. A first antigen binding domain is fused to an alpha chain of TCR and a second antigen binding domain is fused to a beta chain of TCR, or a first antigen binding domain is fused to a gamma chain of TCR and a second antigen binding domain is fused to a delta chain of TCR. FIG. 2K shows a modified TCR complex comprising a first antigen binding domain fused to a second antigen binding domain which in turn in fused to a delta chain of CD3. FIG. 2L shows a modified TCR complex comprising a first antigen binding domain fused to a second antigen binding domain which in turn in fused to a gamma chain of CD3. FIG. 2M shows a modified TCR complex comprising a first antigen binding domain fused to a second antigen binding domain which in turn in fused to an alpha chain of TCR or a gamma chain of TCR. FIG. 2N shows a modified TCR complex comprising a TCR comprising a first antigen binding domain fused to a second antigen binding domain which in turn in fused to a beta chain of TCR or a delta chain of TCR. FIG. 2O shows a modified TCR complex comprising two antigen binding domains. A first antigen binding domain is fused to an epsilon chain of CD3, and a second antigen binding domain is fused to a delta chain of CD3. FIG. 2P shows a modified TCR complex comprising two antigen binding domains. A first antigen binding domain is fused to a delta chain of CD3, and a second antigen binding domain is fused to a gamma chain of CD3. FIG. 2Q shows a modified TCR complex comprising two antigen binding domains. A first antigen binding domain is fused to an alpha chain of TCR or a gamma chain of TCR, and a second antigen binding domain is fused to an epsilon chain of CD3. FIG. 2R shows a modified TCR complex comprising two antigen binding domains. A first antigen binding domain is fused to a beta chain of TCR or a delta chain of TCR, and a second antigen binding domain is fused to an epsilon chain of CD3. FIG. 2S shows a modified TCR complex comprising two antigen binding domains. A first antigen binding domain is fused to an alpha chain of TCR or a gamma chain of TCR, and a second antigen binding domain is fused to a gamma chain of CD3. FIG. 2T shows a modified TCR complex comprising two antigen binding domains. A first antigen binding domain is fused to a beta chain of TCR or a delta chain of TCR, and a second antigen binding domain is fused to a gamma chain of CD3. FIG. 2U shows a modified TCR complex comprising two antigen binding domains. A first antigen binding domain is fused to an alpha chain of TCR or a gamma chain of TCR, and a second antigen binding domain is fused to a delta chain of CD3. FIG. 2V shows a modified TCR complex comprising two antigen binding domains. A first antigen binding domain is fused to a beta chain of TCR or a delta chain of TCR, and a second antigen binding domain is fused to a delta chain of CD3.

FIG. 3A shows a modified TCR complex comprising a first antigen binding domain fused to a second antigen binding domain which is in turn fused to an epsilon chain of CD3. FIG. 3B shows a modified TCR complex comprising a first antigen binding domain fused to a second antigen binding domain which is in turn fused to a delta chain of CD3. FIG. 3C shows a modified TCR complex comprising a first antigen binding domain fused to a second antigen binding domain which is in turn fused to a gamma chain of CD3. FIG. 3D shows a modified TCR complex comprising a first antigen binding domain fused to a second antigen binding domain fused to a third antigen binding domain which is in turn fused to an epsilon chain of CD3. FIG. 3E shows a modified TCR complex comprising a first antigen binding domain fused to a second antigen binding domain fused to a third antigen binding domain which is in turn fused to an delta chain of CD3. FIG. 3F shows a modified TCR complex comprising a first antigen binding domain fused to a second antigen binding domain fused to a third antigen binding domain which is in turn fused to an gamma chain of CD3. FIG. 3G shows a modified TCR complex comprising a first antigen binding domain fused to a second antigen binding domain which is in turn fused to an epsilon chain of CD3, and also comprises a third antigen binding domain fused to a fourth antigen binding domain which is in turn fused to an delta chain of CD3. FIG. 3H shows a modified TCR complex comprising a first antigen binding domain fused to a second antigen binding domain which is in turn fused to an epsilon chain of CD3, and also comprises a third antigen binding domain fused to a fourth antigen binding domain which is in turn fused to a gamma chain of CD3. FIG. 3I shows a modified TCR complex comprising a first antigen binding domain fused to a second antigen binding domain which is in turn fused to a delta chain of CD3, and also comprises a third antigen binding domain fused to a fourth antigen binding domain which is in turn fused to a gamma chain of CD3. FIG. 3J shows a modified TCR complex comprising a first antigen binding domain fused to a second antigen binding domain which is in turn fused to an epsilon chain of CD3, and also comprises a third antigen binding domain fused to a gamma chain of CD3. FIG. 3K shows a modified TCR complex comprising a first antigen binding domain fused to a second antigen binding domain fused to a third antigen binding domain which is in turn fused to an epsilon chain of CD3, and also comprises the fourth antigen binding domain fused to the fifth antigen binding domain fused to the sixth antigen binding domain which is in turn fused to a delta chain of CD3. FIG. 3L shows a modified TCR complex comprising a first antigen binding domain fused to a second antigen binding domain fused to a third antigen binding domain which is in turn fused to an epsilon chain of CD3, and also comprises a fourth antigen binding domain fused to a fifth antigen binding domain fused to a sixth antigen binding domain which is in turn fused to a gamma chain of CD3. FIG. 3M shows a modified TCR complex comprising a first antigen binding domain fused to a second antigen binding domain fused to a third antigen binding domain which is in turn fused to a delta chain of CD3, and also comprises a fourth antigen binding domain fused to a fifth antigen binding domain fused to a sixth antigen binding domain which is in turn fused to a gamma chain of CD3.

FIG. 4A shows a modified TCR complex comprising a first antigen binding domain fused to an epsilon chain of CD3, and a CAR comprising a second antigen binding domain fused to a transmembrane domain and an intracellular signaling domain devoid of CD3-zeta signaling chain. FIG. 4B shows a modified TCR complex comprising a first antigen binding domain fused to a delta chain of CD3, and a CAR comprising a second antigen binding domain fused to a transmembrane domain and an intracellular signaling domain devoid of CD3-zeta signaling chain. FIG. 4C shows a modified TCR complex comprising a first antigen binding domain fused to a gamma chain of CD3, and a CAR comprising a second antigen binding domain fused to a transmembrane domain and an intracellular signaling domain devoid of CD3-zeta signaling chain. FIG. 4D shows a modified TCR complex comprising a first antigen binding domain fused to a second antigen binding domain which is in turn fused to an epsilon chain of CD3, and a CAR comprising a third antigen binding domain fused to a transmembrane domain and an intracellular signaling domain devoid of CD3-zeta signaling chain. FIG. 4E shows a modified TCR complex comprising a first antigen binding domain fused to a second antigen binding domain which is in turn fused to a delta chain of CD3, and a CAR comprising a third antigen binding domain fused to a transmembrane domain and an intracellular signaling domain devoid of CD3-zeta signaling chain. FIG. 4F shows a modified TCR complex comprising a first antigen binding domain fused to a second antigen binding domain which is in turn fused to a gamma chain of CD3, and a CAR comprising a third antigen binding domain fused to a transmembrane domain and an intracellular signaling domain devoid of CD3-zeta signaling chain. FIG. 4G shows a modified TCR complex comprising a first antigen binding domain fused to an epsilon chain of CD3, and a CAR comprising a second antigen binding domain fused to a third antigen binding domain which is in turn fused to a transmembrane domain and an intracellular signaling domain devoid of CD3-zeta signaling chain. FIG. 4H shows a modified TCR complex comprising a first antigen binding domain fused to a delta chain of CD3, and a CAR comprising a second antigen binding domain fused to a third antigen binding domain which is in turn fused to a transmembrane domain and an intracellular signaling domain devoid of CD3-zeta signaling chain. FIG. 4I shows a modified TCR complex comprising a first antigen binding domain fused to a gamma chain of CD3, and a CAR comprising a second antigen binding domain fused to a third antigen binding domain which is in turn fused to a transmembrane domain and an intracellular signaling domain devoid of CD3-zeta signaling chain. FIG. 4J shows a modified TCR complex comprising a first antigen binding domain fused to a second antigen binding domain which is in turn fused to an epsilon chain of CD3, and a CAR comprising a third antigen binding domain fused to a fourth antigen binding domain which is in turn fused to a transmembrane domain and an intracellular signaling domain devoid of CD3-zeta signaling chain. FIG. 4K shows a modified TCR complex comprising a first antigen binding domain fused to a second antigen binding domain which is in turn fused to a delta chain of CD3, and a CAR comprising a third antigen binding domain fused to a fourth antigen binding domain which is in turn fused to a transmembrane domain and an intracellular signaling domain devoid of CD3-zeta signaling chain. FIG. 4L shows a modified TCR complex comprising a first antigen binding domain fused to a second antigen binding domain which is in turn fused to a gamma chain of CD3, and a CAR comprising a third antigen binding domain fused to a fourth antigen binding domain which is in turn fused to a transmembrane domain and an intracellular signaling domain devoid of CD3-zeta signaling chain. FIG. 4M shows a modified TCR complex comprising a first antigen binding domain fused to an epsilon chain of CD3, a second antigen binding domain fused to a gamma chain of CD3, and a CAR comprising a third antigen binding domain fused to a transmembrane domain and an intracellular signaling domain devoid of CD3-zeta signaling chain. FIG. 4N shows a modified TCR complex comprising a first antigen binding domain fused to an epsilon chain of CD3, a second antigen binding domain fused to a delta chain of CD3, and a CAR comprising a third antigen binding domain fused to a transmembrane domain and an intracellular signaling domain devoid of CD3-zeta signaling chain. FIG. 4O shows a modified TCR complex comprising a first antigen binding domain fused to a delta chain of CD3, a second antigen binding domain fused to a gamma chain of CD3, and a CAR comprising a third antigen binding domain fused to a transmembrane domain and an intracellular signaling domain devoid of CD3-zeta signaling chain. FIG. 4P shows a modified TCR complex comprising a first antigen binding domain fused to an epsilon chain of CD3, a second antigen binding domain fused to a gamma chain of CD3, and a CAR comprising a third antigen binding domain fused to a fourth antigen binding domain which is in turn fused to a transmembrane domain and an intracellular signaling domain devoid of CD3-zeta signaling chain. FIG. 4Q shows a modified TCR complex comprising a first antigen binding domain fused to an epsilon chain of CD3, a second antigen binding domain fused to a delta chain of CD3, and a CAR comprising a third antigen binding domain fused to a fourth antigen binding domain which is in turn fused to a transmembrane domain and an intracellular signaling domain devoid of CD3-zeta signaling chain. FIG. 4R shows a modified TCR complex comprising a first antigen binding domain fused to a delta chain of CD3, a second antigen binding domain fused to a gamma chain of CD3, and a CAR comprising a third antigen binding domain fused to a fourth antigen binding domain which is in turn fused to a transmembrane domain and an intracellular signaling domain devoid of CD3-zeta signaling chain. FIG. 4S shows a modified TCR complex comprising a first antigen binding domain fused to a second antigen binding domain which is in turn fused to an epsilon chain of CD3, a third antigen binding domain fused to a gamma chain of CD3, and a CAR comprising a fourth antigen binding domain fused to a fifth antigen binding domain which is in turn fused to a transmembrane domain and an intracellular signaling domain devoid of CD3-zeta signaling chain. FIG. 4T shows a modified TCR complex comprising a first antigen binding domain fused to a second antigen binding domain which is in turn fused to an epsilon chain of CD3, a third antigen binding domain fused to a delta chain of CD3, and a CAR comprising a fourth antigen binding domain fused to a fifth antigen binding domain which is in turn fused to a transmembrane domain and an intracellular signaling domain devoid of CD3-zeta signaling chain. FIG. 4U shows a modified TCR complex comprising a first antigen binding domain fused to a second antigen binding domain which is in turn fused to a gamma chain of CD3, a third antigen binding domain fused to a delta chain of CD3, and a CAR comprising a fourth antigen binding domain fused to a fifth antigen binding domain which is in turn fused to a transmembrane domain and an intracellular signaling domain devoid of CD3-zeta signaling chain. FIG. 4V shows a modified TCR complex comprising a first antigen binding domain fused to a second antigen binding domain which is in turn fused to a gamma chain of CD3, a third antigen binding domain fused to a fourth antigen binding domain which is in turn fused to a delta chain of CD3, and a CAR comprising a fifth antigen binding domain fused to a sixth antigen binding domain which is in turn fused to a transmembrane domain and an intracellular signaling domain devoid of CD3-zeta signaling chain. FIG. 4W shows a modified TCR complex comprising a first antigen binding domain fused to a second antigen binding domain which is in turn fused to an epsilon chain of CD3, a third antigen binding domain fused to a fourth antigen binding domain which is in turn fused to a gamma chain of CD3, and a CAR comprising a fifth antigen binding domain fused to a sixth antigen binding domain which is in turn fused to a transmembrane domain and an intracellular signaling domain devoid of CD3-zeta signaling chain. FIG. 4X shows a modified TCR complex comprising a first antigen binding domain fused to a second antigen binding domain which is in turn fused to an epsilon chain of CD3, a third antigen binding domain fused to a fourth antigen binding domain which is in turn fused to a delta chain of CD3, and a CAR comprising a fifth antigen binding domain fused to a sixth antigen binding domain which is in turn fused to a transmembrane domain and an intracellular signaling domain devoid of CD3-zeta signaling chain.

FIG. 5A shows the anti-tumor cytotoxicity of γδT cells with different molecular designs specified in Example 1 with a focus on the fusion of one or more anti-CLL-1 domains fused to CD3ε subunit of a TCR signaling complex. FIG. 5B shows the anti-tumor cytotoxicity of γδT cells with different molecular designs specified in Example 1 with a focus on the fusion of one or more anti-CLL-1 domains fused to CD3δ subunit of TCR signaling complex. FIG. 5C shows the anti-tumor cytotoxicity of γδT cells with different molecular designs specified in Example 1 with a focus on the fusion of one or more anti-CLL-1 domains fused to CD3γ subunit of a TCR signaling complex. FIG. 5D shows the anti-tumor cytotoxicity of γδT cells with different molecular designs specified in Example 1 with a focus on the parallel design in which two anti-CLL-1 domains are fused to any two of the CD3ε, CD3δ and CD3γ subunit of TCR signaling complex in parallel. FIG. 5E shows the anti-tumor cytotoxicity of αβT cells with different molecular designs specified in Example 1 with a focus on the fusion of one or more anti-CLL-1 domains fused to CD3ε subunit of TCR signaling complex.

FIG. 6A shows GM-CSF production profile of U937-co-cultured γδT cells with different molecular designs specified in Example 1. FIG. 6B shows TNF-α production profile of U937-co-cultured γδT cells with different molecular designs specified in Example 1. FIG. 6C shows IFN-γ production profile of U937-co-cultured γδT cells with different molecular designs specified in Example 1. FIG. 6D shows GM-CSF production profile of U937-co-cultured αβT cells with different molecular designs specified in Example 1. FIG. 6E shows TNF-α production profile of U937-co-cultured αβT cells with different molecular designs specified in Example 1. FIG. 6F shows IFN-γ production profile of U937-co-cultured αβT cells with different molecular designs specified in Example 1.

FIG. 7A shows the colony-forming unit (CFU) assay results of CD34+ cells treated with γδT cells with different molecular designs specified in Example 1. FIG. 7B shows the colony-forming unit (CFU) assay results of CD34+ cells treated with αβT cells with different molecular designs specified in Example 1.

### DETAILED DESCRIPTION

The practice of some methods disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R.I. Freshney, ed. (2010)).

As used in the specification and claims, the singular forms “a,” “an,” and “the” include plural references unless the context clearly dictates otherwise. For example, the term “an antigen binding domain” includes a plurality of antigen binding domains.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, Cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g. a cell can be a synthetically made, sometimes termed an artificial cell).

As used herein, the term “T-cell” or “T lymphocyte” refers to a type of lymphocyte that plays a central role in cell-mediated immunity. T cells can be distinguished from other lymphocytes, such as B cells and natural killer cells, by the presence of a T-cell receptor on the cell surface.

As used herein, the term “T-cell receptor” or “TCR” refers to a molecule on the surface of a T cell or T lymphocyte that is responsible for recognizing an antigen. TCR is a heterodimer which is composed of two different protein chains. In human, 95% of T cells have a TCR consisting of an alpha (α) chain and a beta (β) chain and is referred as αβ TCR. TCR recognizes antigenic peptides degraded from protein bound to major histocompatibility complex molecules (MI-IC) at the cell surface. Meanwhile, 5% of T cells in human have a TCR consisting of a gamma (γ) and a delta (δ) chain and is referred as γδ TCR. Unlike αβ TCR, γδ TCR recognizes peptide and non-peptide antigens in a MHC-independent manner. γδ T cells have shown to play a prominent role in recognizing lipid antigens. In particular, the γ chain of TCR includes but is not limited to Vγ2, Vγ3, Vγ4, Vγ5, Vγ8, Vγ9, Vγ10, a functional variant thereof, and a combination thereof, and the δ chain of TCR includes but is not limited to δ1, δ2, δ3, a functional variant thereof, and a combination thereof. In some examples, the γδ TCR may be Vγ2/Vδ1TCR, Vγ2/Vδ2 TCR, Vγ2/Vδ3 TCR, Vγ3/Vδ1 TCR, Vγ3/Vδ2 TCR, Vγ3/Vδ3 TCR, Vγ4/Vδ1 TCR, Vγ4/Vδ2 TCR, Vγ4/Vδ3 TCR, Vγ5/Vδ1 TCR, Vγ5/Vδ2 TCR, Vγ5/Vδ3 TCR, Vγ8/Vδ1 TCR, Vγ8/Vδ2 TCR, Vγ8/Vδ3 TCR, Vγ9/Vδ1 TCR, Vγ9/Vδ2 TCR, Vγ9/Vδ3 TCR, Vγ10/Vδ1 TCR, Vγ10/Vδ2 TCR, and/or Vγ10/Vδ3 TCR. In some examples, the γδ TCR may be Vγ9/Vδ2 TCR, Vγ10/Vδ2 TCR, and/or Vγ2/Vδ2 TCR.

As used herein, the term “alpha beta T cell”, “αβ T cell” and “AB T cell” can be used interchangeably and refer to a T cell (T lymphocyte) that comprises αβ TCR, or a variant or fragment thereof, whereas the terms “gamma delta T cell,” “γδ T cell,” and “GD T cell” can be used interchangeably and refer to a T cell (T lymphocyte) that comprises γδ TCR, or a variant or fragment thereof, for example, Vγ9δ2 T cells, Vδ1 T cells, Vδ3 T cells or Vδ5 T cells. In some examples, the γδ TCR may be Vγ2/Vδ1T cells, Vγ2/Vδ2 T cells, Vγ2/Vδ3 T cells, Vγ3/Vδ1 T cells, Vγ3/Vδ2 T cells, Vγ3/Vδ3 T cells, Vγ4/Vδ1 T cells, Vγ4/Vδ2 T cells, Vγ4/Vδ3 T cells, Vγ5/Vδ1 T cells, Vγ5/Vδ2 T cells, Vγ5/Vδ3 T cells, Vγ8/Vδ1 T cells, Vγ8/Vδ2 T cells, Vγ8/Vδ3 T cells, Vγ9/Vδ1 T cells, Vγ9/Vδ2 T cells, Vγ9/Vδ3 T cells, Vγ10/Vδ1 T cells, Vγ10/Vδ2 T cells, and/or Vγ10/Vδ3 T cells. In some examples, the γδ T cell may be Vγ9/Vδ2 T cell, Vγ10/Vδ2 T cell, and/or Vγ2/Vδ2 T cell.

The term “activation” and its grammatical equivalents as used herein can refer to a process whereby a cell transitions from a resting state to an active state. This process can comprise a response to an antigen, migration, and/or a phenotypic or genetic change to a functionally active state. For example, the term “activation” can refer to the stepwise process of T cell activation. In some cases, a T cell can require at least two signals to become fully activated. The first signal can occur after engagement of a TCR by the antigen-MHC complex, and the second signal can occur by engagement of co-stimulatory molecules. In some cases, anti-CD3 can mimic the first signal and anti-CD28 can mimic the second signal in vitro.

The term “antigen,” as used herein, refers to a molecule or a fragment thereof capable of being bound by a selective binding agent. As an example, an antigen can be a ligand that can be bound by a selective binding agent such as a receptor. In some cases, the receptor may function as the antigen and the ligand may function as the selective binding agent. As another example, an antigen can be an antigenic molecule that can be bound by a selective binding agent such as an immunological protein (e.g., an antibody). In some cases, the immunological protein may serve as the antigen and the antigenic molecule may serve as the selective binding agent. An antigen can also refer to a molecule or fragment thereof capable of being used in an animal to produce antibodies capable of binding to that antigen.

The term “epitope” and its grammatical equivalents, as used herein, can refer to a part of an antigen that can be recognized by an antigen binding domain. Antigen binding domains can comprise, for example, proteins (e.g., antibodies, antibody fragments) present on a surface, for example a cell surface (e.g., B cells, T cells, CAR-T cells, or engineered cells). For example, an epitope can be a cancer epitope that is recognized by a TCR. Multiple epitopes within an antigen can also be recognized. The epitope can also be mutated.

The term “antibody,” as used herein, refers to a proteinaceous binding molecule with immunoglobulin-like functions. The term antibody includes antibodies (e.g., monoclonal and polyclonal antibodies), as well as derivatives, variants, and fragments thereof. Antibodies include, but are not limited to, immunoglobulins (Ig's) of different classes (i.e. IgA, IgG, IgM, IgD and IgE) and subclasses (such as IgG1, IgG2, etc.). A derivative, variant or fragment thereof can refer to a functional derivative or fragment which retains the binding specificity (e.g., complete and/or partial) of the corresponding antibody. Antigen-binding fragments include Fab, Fab′, F(ab′)₂, variable fragment (Fv), single chain variable fragment (scFv), minibodies, diabodies, and single-domain antibodies (“sdAb” or “nanobodies” or “camelids”). The term antibody includes antibodies and antigen-binding fragments of antibodies that have been optimized, engineered or chemically conjugated. Examples of antibodies that have been optimized include affinity-matured antibodies. Examples of antibodies that have been engineered include Fc optimized antibodies (e.g., antibodies optimized in the fragment crystallizable region) and multispecific antibodies (e.g., bispecific antibodies).

The term “antigen binding domain,” as used herein, refers to a protein or fragment thereof capable of binding an antigen or an epitope. As an example, an antigen binding domain can be a cellular receptor. As an example, an antigen binding domain can be an engineered cellular receptor. As an example, an antigen binding domain can be a soluble receptor. In some cases, an antigen binding domain can be the ligand which is bound by the cellular receptor, the engineered cellular receptor, and/or the soluble receptor.

The term “autologous” and its grammatical equivalents, as used herein, can refer to origination from the same being. For example, an autologous sample (e.g., cells) can refer to a sample which is removed, processed, and then given back to the same subject (e.g., patient) at a later time. Autologous, with respect to a process, can be distinguished from an allogenic process in which the donor of a sample (e.g., cells) and the recipient of the sample are not the same subject.

The terms “cancer neo-antigen,” “neo-antigen,” and “neo-epitope” and their grammatical equivalents, as used herein, can refer to antigens that are not encoded in a normal, non-mutated host genome. A “neo-antigen” can, in some instances, represent either oncogenic viral proteins or abnormal proteins that arise as a consequence of somatic mutations. For example, a neo-antigen can arise by the disruption of cellular mechanisms through the activity of viral proteins. As another example, a neo-antigen can arise from exposure to a carcinogenic compound, which in some cases can lead to a somatic mutation. This somatic mutation can lead to the formation of a tumor/cancer.

The term “cytotoxicity,” as used herein, refers to an unintended or undesirable alteration in the normal state of a cell. The normal state of a cell may refer to a state that is manifested or exists prior to the cell's exposure to a cytotoxic composition, agent and/or condition. A cell that is in a normal state can be in homeostasis. An unintended or undesirable alteration in the normal state of a cell can be manifested in the form of, for example, cell death (e.g., programmed cell death), a decrease in replicative potential, a decrease in cellular integrity such as membrane integrity, a decrease in metabolic activity, a decrease in developmental capability, or any of the cytotoxic effects disclosed herein.

The phrases “reducing cytotoxicity” and “reduce cytotoxicity,” as used herein, refer to a reduction in degree or frequency of unintended or undesirable alterations in the normal state of a cell upon exposure to a cytotoxic composition, agent and/or condition. The phrase can refer to reducing the degree of cytotoxicity in an individual cell that is exposed to a cytotoxic composition, agent and/or condition, or to reducing the number of cells of a population that exhibit cytotoxicity when the population of cells is exposed to a cytotoxic composition, agent and/or condition.

The term “expression” refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell.

The terms “derivative,” “variant,” and “fragment,” when used herein with reference to a polypeptide, refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide.

The term “devoid” or “devoid of” as used herein refers to substantial lack or absence of a component of interest, or substantial lack or absence of a function of a component of interest. The term “devoid” or “devoid of” as used herein include the presence of a component of interest or a structural equivalent thereof, but the function of the present component or its equivalent is substantially lacking or absent.

The term “heterologous” as used herein refers to DNA sequences, protein sequences, or other material that does not naturally exist in a host cell or organism. For example, the term “heterologous sequence” refers to a nucleotide sequence or protein sequence that does not exist in a host cell in nature. “Heterologous expression” refers to the expression of a gene or part of a gene in a host cell or organism and can be performed by recombinant DNA technology. After being inserted in the host, the gene may be integrated into the host DNA, causing permanent expression, or not integrated, causing transient expression.

The term “percent (%) identity,” as used herein, refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment, for purposes of determining percent identity, can be achieved in various ways that are within the skill in the art, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

The terms “subject,” “individual,” and “patient” are used interchangeably herein to refer to a vertebrate, preferably a mammal such as a human. Mammals include, but are not limited to, murines, simians, humans, farm animals, sport animals, and pets. Tissues, cells and their progeny of a biological entity obtained in vivo or cultured in vitro are also encompassed.

The terms “treatment” and “treating,” as used herein, refer to an approach for obtaining beneficial or desired results including, but not limited to, a therapeutic benefit and/or a prophylactic benefit. For example, a treatment can comprise administering a system or cell population disclosed herein. A therapeutic benefit can refer to any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

A “therapeutic effect” may occur if there is a change in the condition being treated. The change may be positive or negative. For example, a ‘positive effect’ may correspond to an increase in the number of activated T-cells in a subject. In another example, a ‘negative effect’ may correspond to a decrease in the amount or size of a tumor in a subject. A “change” in the condition being treated, may refer to at least 1%, 2%, 3%, 4%, 5%, 6%, 7%, 8%, 9%, 10%, 25%, 50%, 75%, or 100% change in the condition. The change can be based on improvements in the severity of the treated condition in an individual, or on a difference in the frequency of improved conditions in populations of individuals with and without the administration of a therapy. Similarly, a method of the present disclosure may comprise administering to a subject an amount of cells that is “therapeutically effective”. The term “therapeutically effective” should be understood to have a definition corresponding to ‘having a therapeutic effect’.

The term “effective amount” or “therapeutically effective amount” refers to the quantity of a composition, for example a composition comprising immune cells such as lymphocytes (e.g., T lymphocytes and/or NK cells), that is sufficient to result in a desired activity upon administration to a subject in need thereof. The term “therapeutically effective” can refer to a quantity of a composition that is sufficient to delay the manifestation, arrest the progression, relieve or alleviate at least one symptom of a disorder treated by the methods of the present disclosure.

The term “TIL” or tumor infiltrating lymphocyte and its grammatical equivalents, as used herein, can refer to a cell isolated from a tumor. A TIL can be any cell found within a tumor. For example, a TIL can be a cell that has migrated to a tumor. A TIL can be a cell that has infiltrated a tumor. A TIL can be a T cell, B cell, monocyte, natural killer (NK) cell, or any combination thereof. A TIL can be a mixed population of cells. A population of TILs can comprise cells of different phenotypes, cells of different degrees of differentiation, cells of different lineages, or any combination thereof.

In an aspect, the present disclosure provides a system for inducing activity of an immune cell and/or a target cell. The system comprises (a) a chimeric antigen receptor (CAR) comprising a first antigen binding domain that exhibits specific binding to a first epitope, a transmembrane domain, and an intracellular signaling domain devoid of a signaling domain of CD3 zeta 0; (b) a modified T cell receptor (TCR) complex comprising a second antigen binding domain which exhibits specific binding to a second epitope, wherein the second antigen binding domain is linked to at least one of (i) at least one TCR chain selected from an alpha chain, a beta chain, a gamma chain and a delta chain of a T cell receptor; (ii) an epsilon chain, a delta chain, and/or a gamma chain of a cluster of differentiation 3 (CD3); or (iii) a CD3 zeta chain. In some embodiments, the second antigen binding domain is linked to at least one of (i) an alpha chain and/or a beta chain of a T cell receptor; (ii) an epsilon chain, a delta chain, and/or a gamma chain of a cluster of differentiation 3 (CD3); or (iii) a CD3 zeta chain. In some embodiments, the second antigen binding domain is linked to at least one of (i) a gamma chain and/or a delta chain of a T cell receptor; (ii) an epsilon chain, a delta chain, and/or a gamma chain of a cluster of differentiation 3 (CD3); or (iii) a CD3 zeta chain.

A chimeric antigen receptor (CAR) of a subject system can comprise a first antigen binding domain that exhibits specific binding to a first epitope. The first antigen binding domain can comprise any protein or molecule that can bind to an epitope. Non-limiting examples of the first antigen binding domain include, but are not limited to, a monoclonal antibody, a polyclonal antibody, a recombinant antibody, a human antibody, a humanized antibody, a murine antibody, or a functional derivative, variant or fragment thereof, including, but not limited to, a Fab, a Fab′, a F(ab′)₂, an Fv, a single-chain Fv (scFv), minibody, a diabody, and a single-domain antibody such as a heavy chain variable domain (VH), a light chain variable domain (VL) and a variable domain (V_{H}H) of camelid derived nanobody. In some embodiments, the first antigen binding domain comprises a single-domain antibody (sdAb). In some embodiments, the first antigen binding domain comprises an sdAb binding to an epitope disclosed herein. In some embodiments, the first antigen binding domain comprises an sdAb selected from anti-CLL-1 sdAb, anti-CD33 sdAb, anti-BCMA sdAb and anti-CD19 sdAb. In some embodiments, the first antigen binding domain comprises a V_{H}H. In some embodiments, the first antigen binding domain comprises a V_{H}H binding to an epitope disclosed herein. In some embodiments, the first antigen binding domain comprises a V_{H}H selected from anti-CLL-1 V_{H}H, anti-CD33 V_{H}H, anti-BCMA V_{H}H and anti-CD19 V_{H}H. In some embodiments, the first antigen binding domain comprises a V_{H}H comprising a sequence selected from SEQ ID NO: 7 to 49. In some embodiments, the first antigen binding domain comprises at least one of a Fab, a Fab′, a F(ab′)₂, an Fv, and an scFv. In some embodiments, the first antigen binding domain comprises an antibody mimetic. Antibody mimetics refer to molecules which can bind a target molecule with an affinity comparable to an antibody, and include single-chain binding molecules, cytochrome b562-based binding molecules, fibronectin or fibronectin-like protein scaffolds (e.g., adnectins), lipocalin scaffolds, calixarene scaffolds, A-domains and other scaffolds. In some embodiments, an antigen binding domain comprises a transmembrane receptor, or any derivative, variant, or fragment thereof. For example, an antigen binding domain can comprise at least a ligand binding domain of a transmembrane receptor. In some embodiments, the antigen binding domain can comprise a scFv. A scFv can be derived from an antibody for which the sequences of the variable regions are known. In some embodiments, a scFv can be derived from an antibody sequence obtained from an available mouse hybridoma. A scFv can be obtained from whole-exomic sequencing of a tumor cell or primary cell. In some embodiments, a scFv can be altered. For instance, a scFv may be modified in a variety of ways. In some cases, a scFv can be mutated, so that the scFv may have higher affinity to its target. In some cases, the affinity of the scFv for its target can be optimized for targets expressed at low levels on normal tissues. This optimization can be performed to minimize potential toxicities, such as cytokine release syndrome. In other cases, the cloning of a scFv that has a higher affinity for the membrane bound form of a target can be preferable over its soluble form counterpart. This modification can be performed if some targets can also be detected in soluble form at different levels and their targeting can cause unintended toxicity, such as cytokine release syndrome.

In some embodiments, the antigen binding domain can comprise one member of an interacting pair. For example, the antigen binding domain may be one member, or a fragment thereof, of an interacting pair comprising a receptor and a ligand. Either the receptor or ligand, or fragments thereof, may be referred to as the antigen binding domain. The other member which is not referred to as the antigen binding domain can comprise the epitope to which the antigen binding domain specifically binds. In some embodiments, the first antigen binding domain and/or the second antigen binding domain comprises a receptor which specifically binds to a ligand. The receptor can comprise G-protein coupled receptors (GPCRs); integrin receptors; cadherin receptors; catalytic receptors including receptors possessing enzymatic activity and receptors which, rather than possessing intrinsic enzymatic activity, act by stimulating non-covalently associated enzymes (e.g., kinases); death receptors such as members of the tumor necrosis factor receptor (TNFR) superfamily; cytokine receptors; immune receptors; and the like. In some embodiments, the first antigen binding domain and/or the second antigen binding domain comprises a ligand which is bound by a receptor.

An antigen binding domain of a CAR of a subject system can be linked to an intracellular signaling domain via a transmembrane domain. A transmembrane domain can be a membrane spanning segment. A transmembrane domain of a subject CAR can anchor the CAR to the plasma membrane of a cell, for example an immune cell. In some embodiments, the membrane spanning segment comprises a polypeptide. The membrane spanning polypeptide linking the antigen binding domain and the intracellular signaling domain of the CAR can have any suitable polypeptide sequence. In some cases, the membrane spanning polypeptide comprises a polypeptide sequence of a membrane spanning portion of an endogenous or wild-type membrane spanning protein. In some embodiments, the membrane spanning polypeptide comprises a polypeptide sequence having at least 1 (e.g., at least 2, 3, 4, 5, 6, 7, 8, 9, 10 or greater) of an amino acid substitution, deletion, and insertion compared to a membrane spanning portion of an endogenous or wild-type membrane spanning protein. In some embodiments, the membrane spanning polypeptide comprises a non-natural polypeptide sequence, such as the sequence of a polypeptide linker. The polypeptide linker may be flexible or rigid. The polypeptide linker can be structured or unstructured. In some embodiments, the membrane spanning polypeptide transmits a signal from an extracellular region of a cell to an intracellular region, for via the antigen binding domain. A native transmembrane portion of CD28 can be used in a CAR. In other cases, a native transmembrane portion of CD8 alpha can also be used in a CAR.

A CAR of a subject system can comprise an intracellular signaling domain. In some embodiments, the intracellular signaling domain of a CAR of a subject system is devoid of a signaling domain, or any derivative, variant, or fragment thereof, involved in immune cell signaling. The signaling domain can induce activity of an immune cell. The signaling domain can transduce the effector function signal and direct the cell to perform a specialized function. The signaling domain can comprise signaling domains of other molecules. In some embodiments, a subject CAR comprises an intracellular signaling domain devoid of a signaling domain of CD3 zeta.

In some embodiments, a subject CAR comprises an intracellular domain devoid of an immune cell signaling domain that can be involved in regulating primary activation of the TCR complex in either a stimulatory way or an inhibitory way. The intracellular signaling domain of a subject CAR can be devoid of a signaling domain of an Fcγ receptor (FcγR), an Fcε receptor (FcεR), an Fcα receptor (FcαR), neonatal Fc receptor (FcRn), CD3, CD3 ζ, CD3 γ, CD3 δ, CD3 ε, CD4, CD5, CD8, CD21, CD22, CD28, CD32, CD40L (CD154), CD45, CD66d, CD79a, CD79b, CD80, CD86, CD278 (also known as ICOS), CD247 ζ, CD247 η, DAP10, DAP12, FYN, LAT, Lck, MAPK, MHC complex, NFAT, NF-κB, PLC-γ, iC3b, C3dg, C3d, and Zap70.

In some cases, the intracellular signaling domain of a subject CAR can be devoid of at least a portion of a signaling domain of TCR. In some cases, the intracellular signaling domain of the subject CAR can be devoid of the entire portion of the signaling domain of TCR. In some cases, the intracellular signaling domain of the subject CAR can be devoid of at least 1%, 2%, 3%, 4%, 5%, 6%, 7%, 8%, 9%, 10%, 20%, 30%, 40%, 50%, 60%, 70%, 80%, 90%, 95%, 99%, or more of the signaling domain of the TCR. In some cases, the intracellular signaling domain of the subject CAR can be devoid of at most 100%, 95%, 90%, 80%, 70%, 60%, 50%, 40%, 30%, 20%, 10%, 9%, 8%, 7%, 6%, 5%, 4%, 3%, 2%, 1%, or less of the signaling domain of the TCR.

In some cases, the intracellular signaling domain of a subject CAR can be devoid of at least one amino acid of a signaling domain of TCR. In some cases, the intracellular signaling domain of the subject CAR can be devoid of the entire amino acid sequence of the signaling domain of TCR. In some cases, the intracellular signaling domain of the subject CAR can be devoid of at least 1%, 2%, 3%, 4%, 5%, 6%, 7%, 8%, 9%, 10%, 20%, 30%, 40%, 50%, 60%, 70%, 80%, 90%, 95%, 99%, or more of the amino acid sequence of the signaling domain of the TCR. In some cases, the intracellular signaling domain of the subject CAR can be devoid of at most 100%, 95%, 90%, 80%, 70%, 60%, 50%, 40%, 30%, 20%, 10%, 9%, 8%, 7%, 6%, 5%, 4%, 3%, 2%, 1%, or less of the amino acid sequence of the signaling domain of the TCR.

In some cases, the intracellular signaling domain of a subject CAR can be devoid of at least a portion of an intracellular domain of TCR. In some cases, the intracellular signaling domain of the subject CAR can be devoid of the entire intracellular domain of TCR. In some cases, the intracellular signaling domain of the subject CAR can be devoid of at least 1%, 2%, 3%, 4%, 5%, 6%, 7%, 8%, 9%, 10%, 20%, 30%, 40%, 50%, 60%, 70%, 80%, 90%, 95%, 99%, or more of the intracellular domain of the TCR. In some cases, the intracellular signaling domain of the subject CAR can be devoid of at most 100%, 95%, 90%, 80%, 70%, 60%, 50%, 40%, 30%, 20%, 10%, 9%, 8%, 7%, 6%, 5%, 4%, 3%, 2%, 1%, or less of the intracellular domain of the TCR.

In some cases, the intracellular signaling domain of a subject CAR can be devoid of at least a one amino acid of an intracellular domain of TCR. In some cases, the intracellular signaling domain of the subject CAR can be devoid of the entire amino acid sequence of the intracellular domain of TCR. In some cases, the intracellular signaling domain of the subject CAR can be devoid of at least 1%, 2%, 3%, 4%, 5%, 6%, 7%, 8%, 9%, 10%, 20%, 30%, 40%, 50%, 60%, 70%, 80%, 90%, 95%, 99%, or more of the amino acid sequence of the intracellular domain of the TCR. In some cases, the intracellular signaling domain of the subject CAR can be devoid of at most 100%, 95%, 90%, 80%, 70%, 60%, 50%, 40%, 30%, 20%, 10%, 9%, 8%, 7%, 6%, 5%, 4%, 3%, 2%, 1%, or less of the amino acid sequence of the intracellular domain of the TCR.

In some embodiments, the intracellular signaling domain of a CAR of a subject system is devoid of an immunoreceptor tyrosine-based activation motif or ITAM. ITAM comprises two repeats of the amino acid sequence YxxL/I separated by 6-8 amino acids, wherein each x is independently any amino acid, producing the conserved motif YxxL/Ix₍₆₋₈₎YxxL/I. ITAM can be modified, for example, by phosphorylation when the antigen binding domain is bound to an epitope. A phosphorylated ITAM can function as a docking site for other proteins, for example proteins involved in various signaling pathways.

In some embodiments, the intracellular signaling domain of a subject CAR is devoid of FcγR signaling domain (e.g., ITAM). The FcγR signaling domain can be selected from FcγRI (CD64), FcγRIIA (CD32), FcγRIIB (CD32), FcγRIIIA (CD16a), and FcγRIIIB (CD16b). In some embodiments, the intracellular signaling domain is devoid of FcεR signaling domain (e.g., ITAM). The FcεR signaling domain can be selected from FcεRI and FcεRII (CD23). In some embodiments, the intracellular signaling domain is devoid of FcαR signaling domain (e.g., ITAM). The FcαR signaling domain can be selected from FcαRI (CD89) and Fcα/μR. In some embodiments, the intracellular signaling domain is devoid of an ITAM of CD3 zeta. In some embodiments, a subject CAR comprises an intracellular signaling domain devoid of CD3 zeta.

In some embodiments, an intracellular signaling domain of a subject CAR is devoid of an immunoreceptor tyrosine-based inhibition motif or ITIM. ITIM can comprise a conserved sequence of amino acids (S/IN/LxYxxI/V/L) that is found in the cytoplasmic tails of some inhibitory receptors of the immune system. ITIM can be modified, for example phosphorylated, by enzymes such as a Src kinase family member (e.g., Lck). Following phosphorylation, other proteins, including enzymes, can be recruited to the ITIM. These other proteins include, but are not limited to, enzymes such as the phosphotyrosine phosphatases SHP-1 and SHP-2, the inositol-phosphatase called SHIP, and proteins having one or more SH2 domains (e.g., ZAP70). A intracellular signaling domain can comprise a signaling domain (e.g., ITIM) of BTLA, CD5, CD31, CD66a, CD72, CMRF35H, DCIR, EPO-R, FcγRIIB (CD32), Fc receptor-like protein 2 (FCRL2), Fc receptor-like protein 3 (FCRL3), Fc receptor-like protein 4 (FCRL4), Fc receptor-like protein 5 (FCRL5), Fc receptor-like protein 6 (FCRL6), protein G6b (G6B), interleukin 4 receptor (IL4R), immunoglobulin superfamily receptor translocation-associated 1 (IRTA1), immunoglobulin superfamily receptor translocation-associated 2 (IRTA2), killer cell immunoglobulin-like receptor 2DL1 (KIR2DL1), killer cell immunoglobulin-like receptor 2DL2 (KIR2DL2), killer cell immunoglobulin-like receptor 2DL3 (KIR2DL3), killer cell immunoglobulin-like receptor 2DL4 (KIR2DL4), killer cell immunoglobulin-like receptor 2DL5 (KIR2DL5), killer cell immunoglobulin-like receptor 3DL1 (KIR3DL1), killer cell immunoglobulin-like receptor 3DL2 (KIR3DL2), leukocyte immunoglobulin-like receptor subfamily B member 1 (LIR1), leukocyte immunoglobulin-like receptor subfamily B member 2 (LIR2), leukocyte immunoglobulin-like receptor subfamily B member 3 (LIR3), leukocyte immunoglobulin-like receptor subfamily B member 5 (LIR5), leukocyte immunoglobulin-like receptor subfamily B member 8 (LIRE), leukocyte-associated immunoglobulin-like receptor 1 (LAIR-1), mast cell function-associated antigen (MAFA), NKG2A, natural cytotoxicity triggering receptor 2 (NKp44), NTB-A, programmed cell death protein 1 (PD-1), PILR, SIGLECL1, sialic acid binding Ig like lectin 2 (SIGLEC2 or CD22), sialic acid binding Ig like lectin 3 (SIGLEC3 or CD33), sialic acid binding Ig like lectin 5 (SIGLEC5 or CD170), sialic acid binding Ig like lectin 6 (SIGLEC6), sialic acid binding Ig like lectin 7 (SIGLEC7), sialic acid binding Ig like lectin 10 (SIGLEC10), sialic acid binding Ig like lectin 11 (SIGLEC11), sialic acid binding Ig like lectin 4 (SIGLEC4), sialic acid binding Ig like lectin 8 (SIGLEC8), sialic acid binding Ig like lectin 9 (SIGLEC9), platelet and endothelial cell adhesion molecule 1 (PECAM-1), signal regulatory protein (SIRP 2), and signaling threshold regulating transmembrane adaptor 1 (SIT).

In some embodiments, the intracellular signaling domain is devoid of both ITAM and ITIM domains.

In some cases, the intracellular signaling domain of a subject CAR can be devoid of at least 1, 2, 3, 4, 5, 6, 7, 8, 9, 10, or more ITAM domains of TCR. In some cases, the intracellular signaling domain of the subject CAR can be devoid of at most 10, 9, 8, 7, 6, 5, 4, 3, 2, or 1 ITAM domain of TCR. In some cases, the intracellular signaling domain of the subject CAR can be devoid of the entire ITAM domains of TCR.

In some cases, the intracellular signaling domain of a subject CAR can include a co-stimulatory domain. In some embodiments, a co-stimulatory domain, for example from co-stimulatory molecule, can provide co-stimulatory signals for immune cell signaling. In some embodiments, a costimulatory domain is operable to regulate a proliferative and/or survival signal in the immune cell. In some embodiments, a co-stimulatory signaling domain comprises a signaling domain of a MHC class I protein, MHC class II protein, TNF receptor protein, immunoglobulin-like protein, cytokine receptor, integrin, signaling lymphocytic activation molecule (SLAM protein), activating NK cell receptor, BTLA, or a Toll ligand receptor. In some embodiments, the costimulatory domain comprises a signaling domain of a molecule selected from the group consisting of: 2B4/CD244/SLAMF4, 4-1BB/TNFSF9/CD137, B7-1/CD80, B7-2/CD86, B7-H1/PD-L1, B7-H2, B7-H3, B7-H4, B7-H6, B7-H7, BAFF R/TNFRSF13C, BAFF/BLyS/TNFSF13B, BLAME/SLAMF8, BTLA/CD272, CD100 (SEMA4D), CD103, CD11a, CD11b, CD11c, CD11d, CD150, CD160 (BY55), CD18, CD19, CD2, CD200, CD229/SLAMF3, CD27 Ligand/TNFSF7, CD27/TNFRSF7, CD28, CD29, CD2F-10/SLAMF9, CD30 Ligand/TNFSF8, CD30/TNFRSF8, CD300a/LMIR1, CD4, CD40 Ligand/TNFSF5, CD40/TNFRSF5, CD48/SLAMF2, CD49a, CD49D, CD49f, CD5, CD53, CD58/LFA-3, CD69, CD7, CD8 α, CD8 β, CD82/Kai-1, CD84/SLAMF5, CD90/Thy1, CD96, CD5, CEACAM1, CRACC/SLAMF7, CRTAM, CTLA-4, DAP12, Dectin-1/CLEC7A, DNAM1 (CD226), DPPIV/CD26, DR3/TNFRSF25, EphB6, GADS, Gi24/VISTA/B7-H5, GITR Ligand/TNFSF18, GITR/TNFRSF18, HLA Class I, HLA-DR, HVEM/TNFRSF14, IA4, ICAM-1, ICOS/CD278, Ikaros, IL2R β, IL2R γ, IL7R α, Integrin α4/CD49d, Integrin α41β1, Integrin α4β7/LPAM-1, IPO-3, ITGA4, ITGA6, ITGAD, ITGAE, ITGAL, ITGAM, ITGAX, ITGB1, ITGB2, ITGB7, KIRDS2, LAG-3, LAT, LIGHT/TNFSF14, LTBR, Ly108, Ly9 (CD229), lymphocyte function associated antigen-1 (LFA-1), Lymphotoxin-α/TNF-β, NKG2C, NKG2D, NKp30, NKp44, NKp46, NKp80 (KLRF1), NTB-A/SLAMF6, OX40 Ligand/TNFSF4, OX40/TNFRSF4, PAG/Cbp, PD-1, PDCD6, PD-L2/B7-DC, PSGL1, RELT/TNFRSF19L, SELPLG (CD162), SLAM (SLAMF1), SLAM/CD150, SLAMF4 (CD244), SLAMF6 (NTB-A), SLAMF7, SLP-76, TACI/TNFRSF13B, TCL1A, TCL1B, TIM-1/KIM-1/HAVCR, TIM-4, TL1A/TNFSF15, TNF RII/TNFRSF1B, TNF-α, TRANCE/RANKL, TSLP, TSLP R, VLA1, and VLA-6. In some embodiments, the intracellular signaling domain comprises multiple costimulatory domains, for example at least two, e.g., at least 3, 4, or 5 costimulatory domains. Co-stimulatory signaling regions may provide a signal synergistic with the primary effector activation signal and can complete the requirements for activation of a T cell. In some embodiments, the addition of co-stimulatory domains to the CAR can enhance the efficacy and persistence of the immune cells provided herein. In some embodiments, the intracellular signaling domain of a subject CAR comprises only a costimulatory domain, which is also referred as “costimulatory only CAR.” In some embodiments, the co-stimulatory domain of the co-stimulatory only CAR comprises a signaling domain of CD27.

In some cases, the intracellular signaling domain of the subject CAR can include at least 1, 2, 3, 4, 5, 6, 7, 8, 9, 10, or more co-stimulatory domains. In some cases, the intracellular signaling domain of the subject CAR can include at most 10, 9, 8, 7, 6, 5, 4, 3, 2, or 1 co-stimulatory domain.

Examples of costimulatory signaling domains are provided in Table 1.

TABLE 1
Intracellular co-stimulatory signaling domains

| Gene | | | NCBI number | | | Location in |
|---|---|---|---|---|---|---|
| Symbol | Abbreviation | Name | (GRCh38.p2) | Start | Stop | genome |
| CD27 | CD27; T14; S152; | CD27 | 939 | 6444885 | 6451718 | 12p13 |
| | Tp55; TNFRSF7; | molecule | | | | |
| | S152. LPFS2 | | | | | |
| CD28 | Tp44; CD28; | CD28 | 940 | 203706475 | 203738912 | 2q33 |
| | CD28 antigen | molecule | | | | |
| TNFRSF9 | ILA; 4-1BB; | tumor necrosis | 3604 | 7915871 | 7943165 | 1p36 |
| | CD137; CDw137 | factor receptor | | | | |
| | | superfamily, | | | | |
| | | member 9 | | | | |
| TNFRSF4 | OX40; ACT35; | tumor necrosis | 7293 | 1211326 | 1214638 | 1p36 |
| | CD134; IMD16; | factor receptor | | | | |
| | TXGP1L | superfamily, | | | | |
| | | member 4 | | | | |
| TNFRSF8 | CD30; Ki-1; | tumor necrosis | 943 | 12063330 | 12144207 | 1p36 |
| | D1S166E | factor receptor | | | | |
| | | superfamily, | | | | |
| | | member 8 | | | | |
| CD40LG | IGM; IMD3; | CD40 ligand | 959 | 136648177 | 136660390 | Xq26 |
| | TRAP; gp39; | | | | | |
| | CD154; CD40L; | | | | | |
| | HIGM1; T-BAM; | | | | | |
| | TNFSF5; hCD40L | | | | | |
| ICOS | AILIM; CD278; | inducible T- | 29851 | 203936731 | 203961579 | 2q33 |
| | CVID1 | cell co- | | | | |
| | | stimulator | | | | |
| ITGB2 | LAD; CD18; | integrin, beta 2 | 3689 | 44885949 | 44928873 | 21q22.3 |
| | MF17; MFI7; | (comp1ement | | | | |
| | LCAMB; LFA-1; | component 3 | | | | |
| | MAC-1 | receptor 3 and | | | | |
| | | 4 subunit) | | | | |
| CD2 | T11; SRBC; | CD2 molecule | 914 | 116754435 | 116769229 | 1p13.1 |
| | LFA-2 | | | | | |
| CD7 | GP40; TP41; | CD7 molecule | 924 | 82314865 | 82317604 | 17q25.2- |
| | Tp40; LEU-9 | | | | | q25.3 |
| KLRC2 | NKG2C; | killer cell | 3822 | 10430599 | 10435993 | 12p13 |
| | CD159c; NKG2-C | lectin-like | | | | |
| | | receptor | | | | |
| | | subfamily C, | | | | |
| | | member 2 | | | | |
| TNFRSF18 | AITR; GITR; | tumor necrosis | 8784 | 1203508 | 1206709 | 1p36.3 |
| | CD357; GITR-D | factor receptor | | | | |
| | | superfamily, | | | | |
| | | member 18 | | | | |
| TNFRSF14 | TR2; ATAR; | tumor necrosis | 8764 | 2556365 | 2565622 | 1p36.32 |
| | HVEA; HVEM; | factor receptor | | | | |
| | CD270; LIGHTR | superfamily, | | | | |
| | | member 14 | | | | |
| HAVCR1 | TIM; KIM1; | hepatitis A | 26762 | 156979480 | 157069527 | 5q33.2 |
| | TIM1; CD365; | virus cellular | | | | |
| | HAVCR; KIM-1; | receptor 1 | | | | |
| | TIM-1; TIMD1; | | | | | |
| | TIMD-1; HAVCR-1 | | | | | |
| LGALS9 | HUAT; | lectin, | 3965 | 27631148 | 27649560 | 17q11.2 |
| | LGALS9A, | galactoside- | | | | |
| | Galectin-9 | binding, | | | | |
| | | soluble, 9 | | | | |
| CD83 | BL11; HB15 | CD83 | 9308 | 14117256 | 14136918 | 6p23 |
| | | molecule | | | | |
| DAP10 | DAP10; KAP10; | hematopoietic | 10870 | 35902365 | 35904271 | 19q13.12 |
| | PIK3AP | cell signal | | | | |
| | | transducer | | | | |

In some embodiments, the intracellular domain of a subject CAR is devoid of a signaling domain and comprises a co-stimulatory domain. As an example, a CAR is devoid of a CD-3 zeta domain and comprises a single co-stimulatory domain such as CD27, CD28 or 4-1BB. As another example, a CAR is devoid of CD-3 zeta domain and comprises two co-stimulatory domains, such as CD28/OX40 or CD28/4-1BB. As another example, a CAR is devoid of a CD3 zeta domain and comprises more than two stimulatory domains. In some embodiments, the intracellular domain of a subject CAR is devoid of an ITAM and comprises a co-stimulatory domain. As an example, a CAR is devoid of an ITAM and comprises a single co-stimulatory domain such as CD27, CD28 or 4-1BB. As another example, a CAR is devoid of an ITAM and comprises two co-stimulatory domains, such as CD28/OX40 or CD28/4-1BB. As another example, a CAR is devoid of an ITAM and comprises more than two stimulatory domains.

In some embodiments, a subject CAR may not be configured to form a complex with one another. In some embodiments, a subject CAR may be configured to form a complex with one another as a multimeric structure. In some cases, the subject CAR may be configured to form at least a (1) monomer, (2) dimer, (3) trimer, (4) tetramer, (5) pentamer, (6) hexamer, (7) heptamer, (8) octamer, (9) decamer, (10) dodecamer, or (11) higher multimer. In some cases, the subject CAR may be configured to form a (1) monomer, (2) dimer, (3) trimer, (4) tetramer, (5) pentamer, (6) hexamer, (7) heptamer, (8) octamer, (9) decamer, (10) dodecamer, and/or (11) higher multimer. In some cases, the subject CAR may be configured to form a (1a) homo-dimer and/or (1b) hetero-dimer; (2a) homo-trimer and/or (2b) hetero-trimer; (3a) homo-tetramer and/or (3b) hetero-tetramer; (4a) homo-pentamer and/or (4b) hetero-pentamer; (5a) homo-hexamer and/or (5b) hetero-hexamer; (6a) homo-octamer and/or (6b) hetero-octamer; (7a) homo-decamer and/or (7b) hetero-decamer; and/or (8a) homo-dodecamer and/or (8b) hetero-dodecamer.

In some embodiments, a subject CAR can comprise a hinge or a spacer. The hinge or the spacer can refer to a segment between the antigen binding domain and the transmembrane domain. In some embodiments, a hinge can be used to provide flexibility to an antigen binding domain, e.g., scFv. In some embodiments, a hinge can be used to detect the expression of a CAR on the surface of a cell, for example when antibodies to detect the scFv are not functional or available. In some cases, the hinge is derived from an immunoglobulin molecule and may require optimization depending on the location of the first epitope or second epitope on the target. In some cases, a hinge may not belong to an immunoglobulin molecule but instead to another molecule such the native hinge of a CD8 alpha molecule. A CD8 alpha hinge can contain cysteine and proline residues which may play a role in the interaction of a CD8 co-receptor and MHC molecule. In some embodiments, a cysteine and proline residue can influence the performance of a CAR and may therefore be engineered to influence a CAR performance.

A hinge can be of any suitable length. In some embodiments, a CAR's hinge can be size tunable and can compensate to some extent in normalizing the orthogonal synapse distance between a CAR expressing cell and a target cell. This topography of the immunological synapse between the CAR expressing cell and target cell can also define a distance that cannot be functionally bridged by a CAR due to a membrane-distal epitope on a cell-surface target molecule that, even with a short hinge CAR, cannot bring the synapse distance in to an approximation for signaling. Likewise, membrane-proximal CAR target antigen epitopes have been described for which signaling outputs are only observed in the context of a long hinge CAR. A hinge disclosed herein can be tuned according to the single chain variable fragment region that can be used.

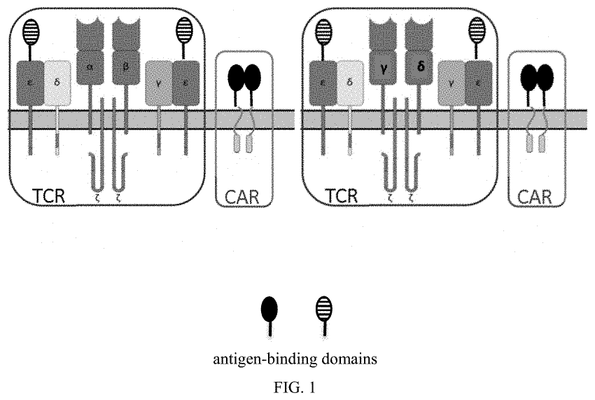

As an example, a CAR can comprise an extracellular antigen binding domain, a transmembrane domain, and an intracellular signaling domain, as illustrated in FIG. 1. A CAR may generally comprise an antigen binding domain derived from single chain antibody, hinge domain (H) or spacer, transmembrane domain (TM) providing anchorage to plasma membrane and signaling domains devoid of a CD3 zeta chain. A CAR can be devoid of a CD3 zeta chain and comprise a costimulatory domain, such as CD27 or 4-1BB. A CAR can be devoid of a CD3 zeta chain and comprise at least two costimulatory domains, such as 4-1BB, and OX40. Various combinations of costimulatory domains such as 4-1BB, OX40, CD28, CD27 and the like may be utilized in a subject CAR.

A modified T cell receptor (TCR) complex of a subject system can comprise a second antigen binding domain which exhibits binding to a second epitope. The second antigen binding domain can comprise any protein or molecule that can bind to an epitope. In some embodiments, the second antigen binding domain comprises a heterologous sequence exhibiting binding to the second epitope. Non-limiting examples of the second antigen binding domain of the TCR complex include, but are not limited to, a monoclonal antibody, a polyclonal antibody, a recombinant antibody, a human antibody, a humanized antibody, or a functional derivative, variant or fragment thereof, including, but not limited to, a Fab, a Fab′, a F(ab′)₂, an Fv, a single-chain Fv (scFv), minibody, a diabody, and a single-domain antibody such as a heavy chain variable domain (VH), a light chain variable domain (VL) and a variable domain (V_{H}H) of camelid derived Nanobody. In some embodiments, the second antigen binding domain comprises a single-domain antibody (sdAb). In some embodiments, the second antigen binding domain comprises an sdAb binding to an epitope disclosed herein. In some embodiments, the second antigen binding domain comprises an sdAb selected from anti-CLL-1 sdAb, anti-CD33 sdAb, anti-BCMA sdAb and anti-CD19 sdAb. In some embodiments, the second antigen binding domain comprises a V_{H}H. In some embodiments, the second antigen binding domain comprises a V_{H}H binding to an epitope disclosed herein. In some embodiments, the second antigen binding domain comprises a V_{H}H selected from anti-CLL-1 V_{H}H, anti-CD33 V_{H}H, anti-BCMA V_{H}H and anti-CD19 V_{H}H. In some embodiments, the second antigen binding domain comprises a V_{H}H comprising a sequence selected from SEQ ID NO: 7 to 49. In some embodiments, the second antigen binding domain of the TCR complex comprises at least one of a Fab, a Fab′, a F(ab′)2, an Fv, and a scFv. In some embodiments, the second antigen binding domain of the TCR complex comprises an antibody mimetic. Antibody mimetics refer to molecules which can bind a target molecule with an affinity comparable to an antibody, and include single-chain binding molecules, cytochrome b562-based binding molecules, fibronectin or fibronectin-like protein scaffolds (e.g., adnectins), lipocalin scaffolds, calixarene scaffolds, A-domains and other scaffolds. In some embodiments, an antigen binding domain comprises a transmembrane receptor, or any derivative, variant, or fragment thereof. For example, an antigen binding domain can comprise at least a ligand binding domain of a transmembrane receptor.

In some embodiments, the antigen binding domain can comprise one member of an interacting pair. For example, the antigen binding domain may be one member, or a fragment thereof, of an interacting pair comprising a receptor and a ligand. Either the receptor or ligand, or fragments thereof, may be referred to as the antigen binding domain. The other member which is not referred to as the antigen binding domain can comprise the epitope to which the antigen binding domain specifically binds.

The second antigen binding domain can be linked to any member of the TCR complex, and the TCR may be an alpha/beta or gamma/delta TCR. The second antigen binding domain can be linked to at least one of a TCR chain, a cluster of differentiation 3 (CD3) chain, or CD3 zeta chain. The second antigen binding domain can be linked to transmembrane receptor of a TCR, for example, TCR-delta, TCR-gamma, TCR-alpha, or TCR-beta. The second antigen binding domain can be linked to a CD3 chain, for example, CD3-epsilon, CD3-delta, or CD3-gamma. The second antigen binding domain can be linked to CD3 zeta chain.

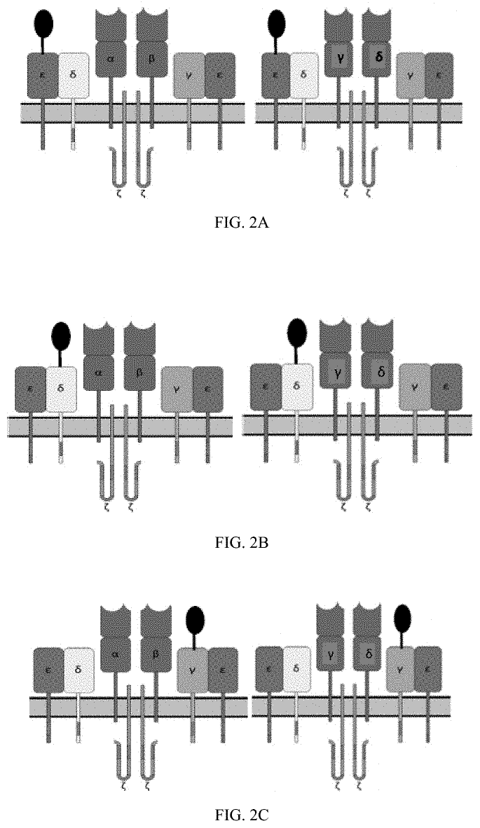

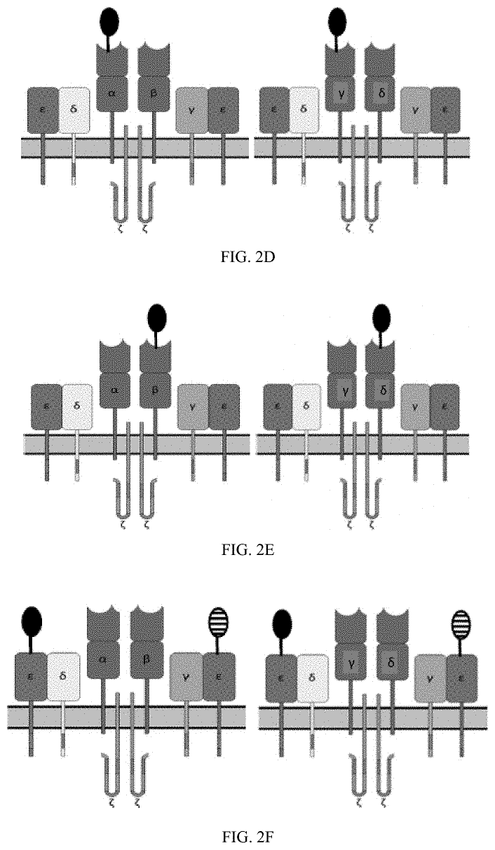

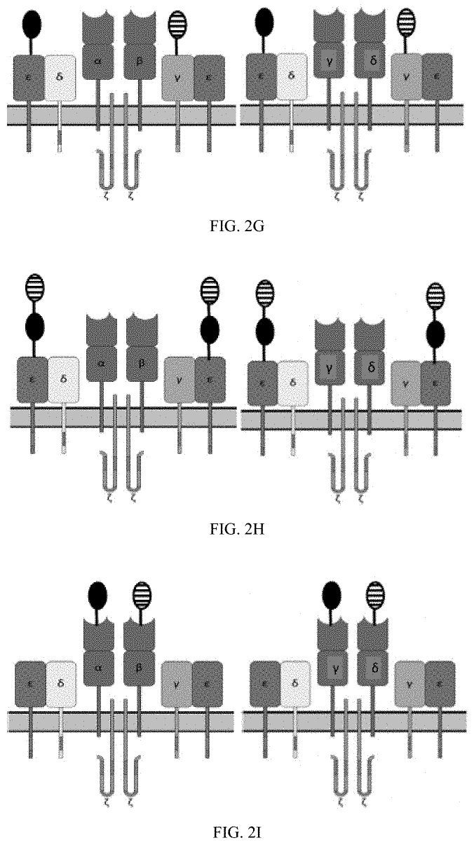

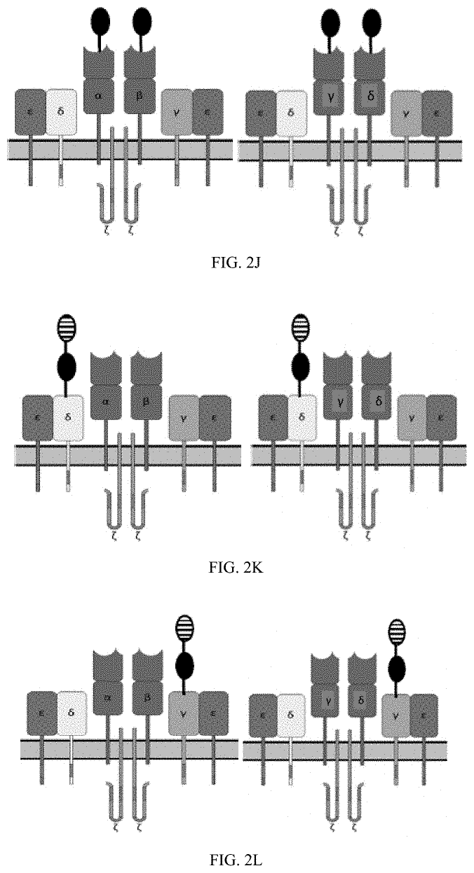

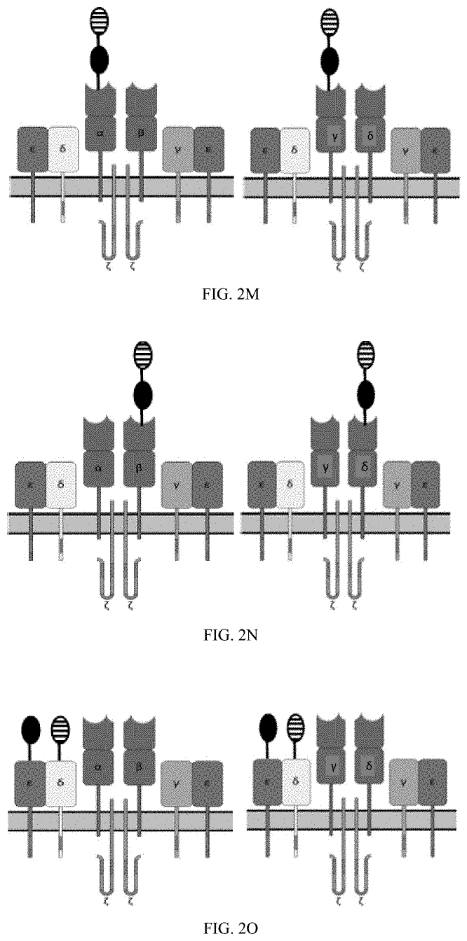

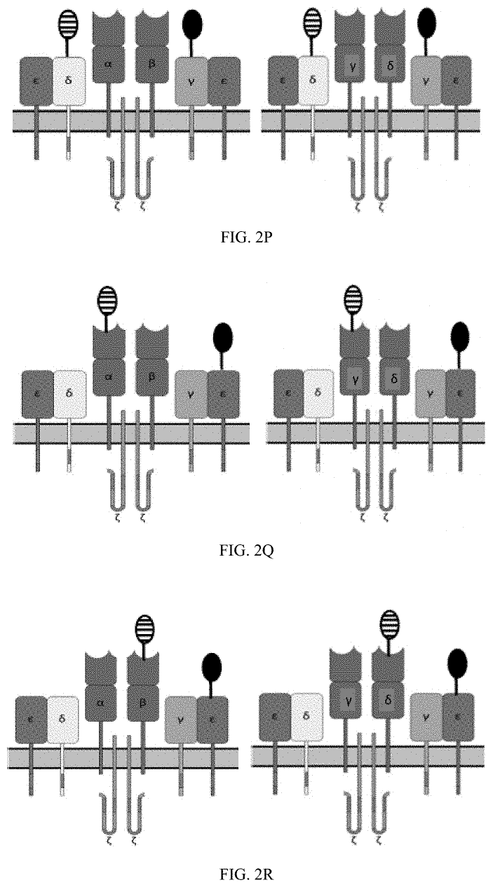

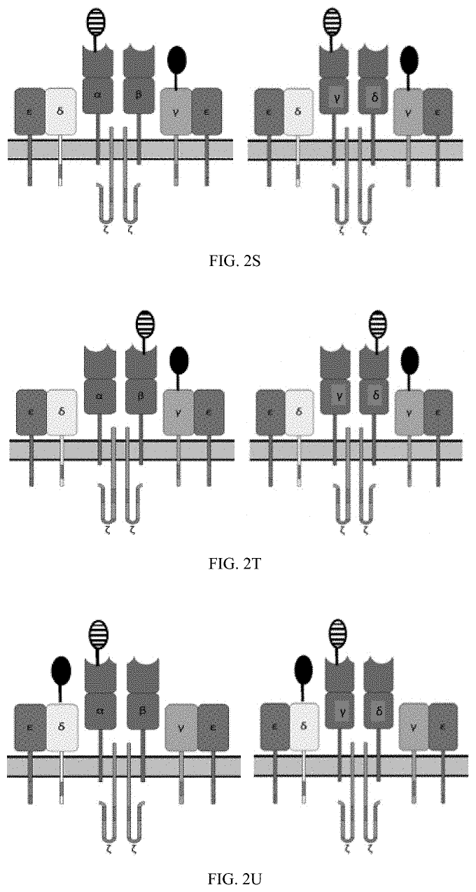

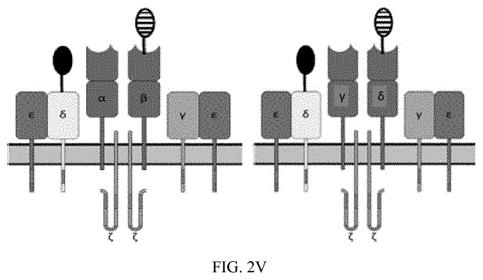

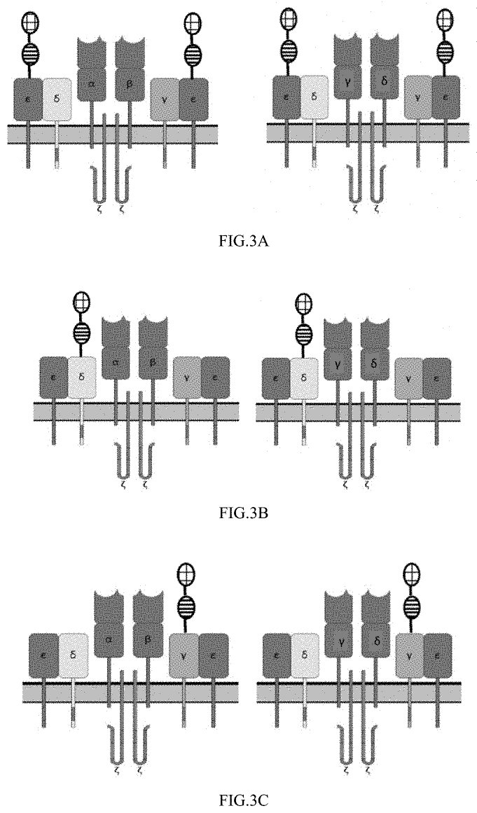

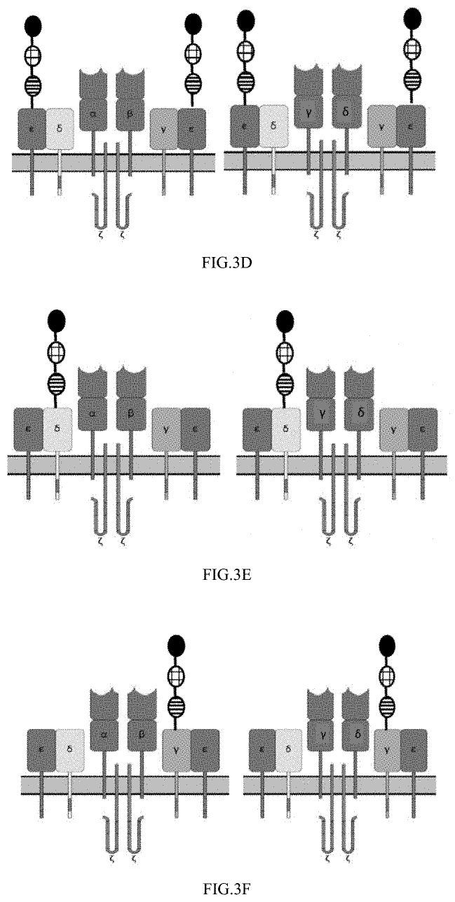

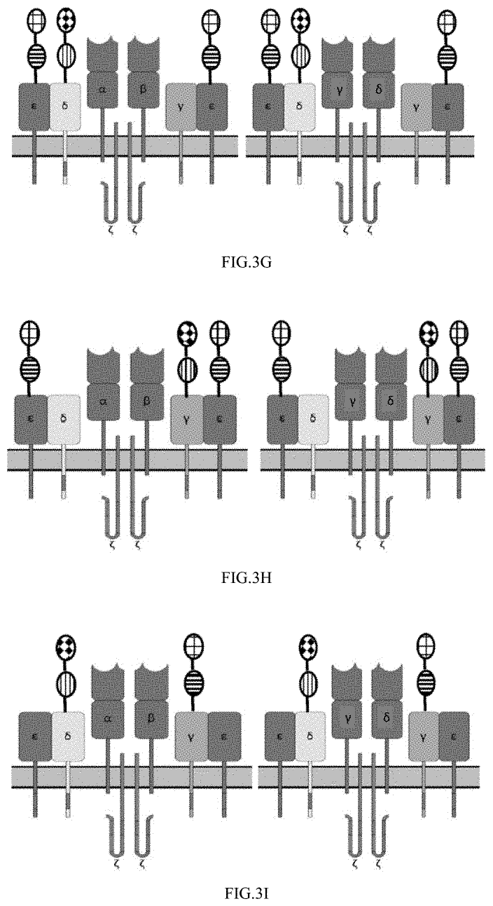

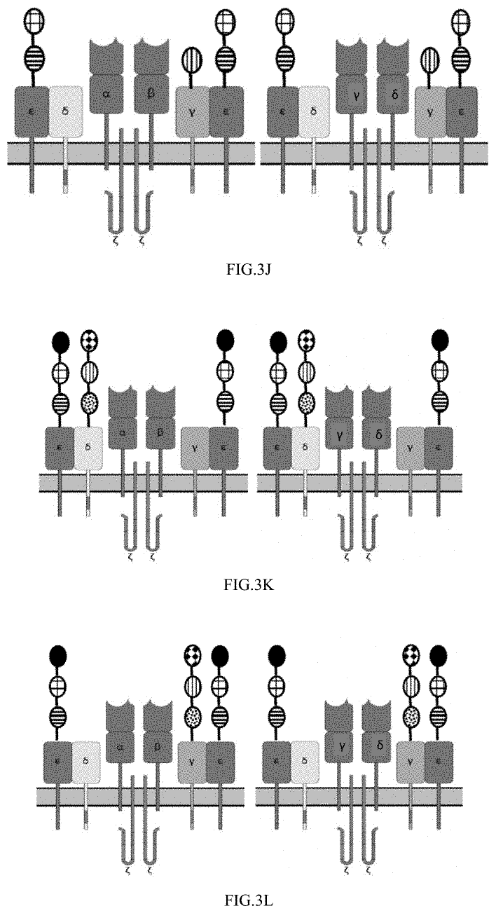

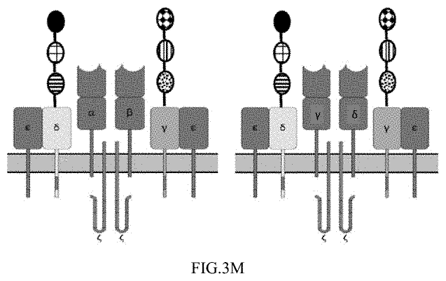

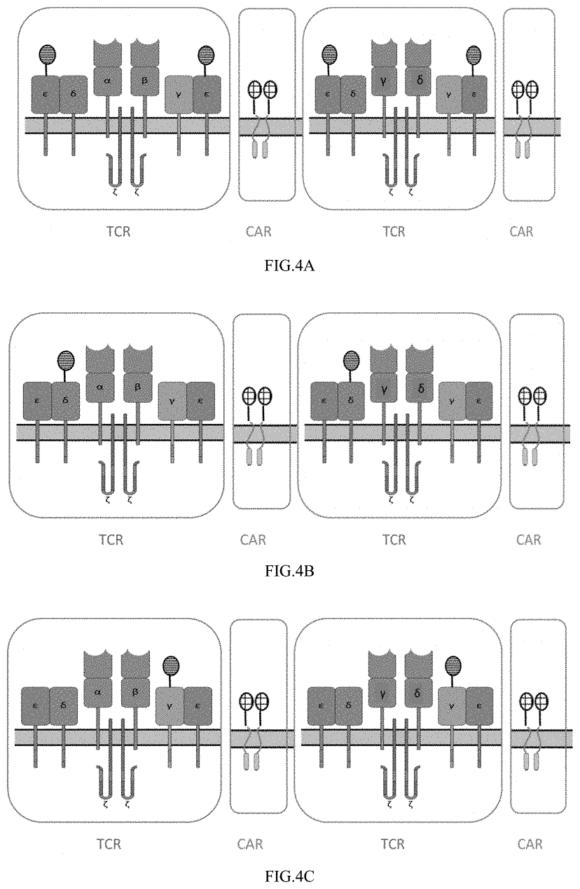

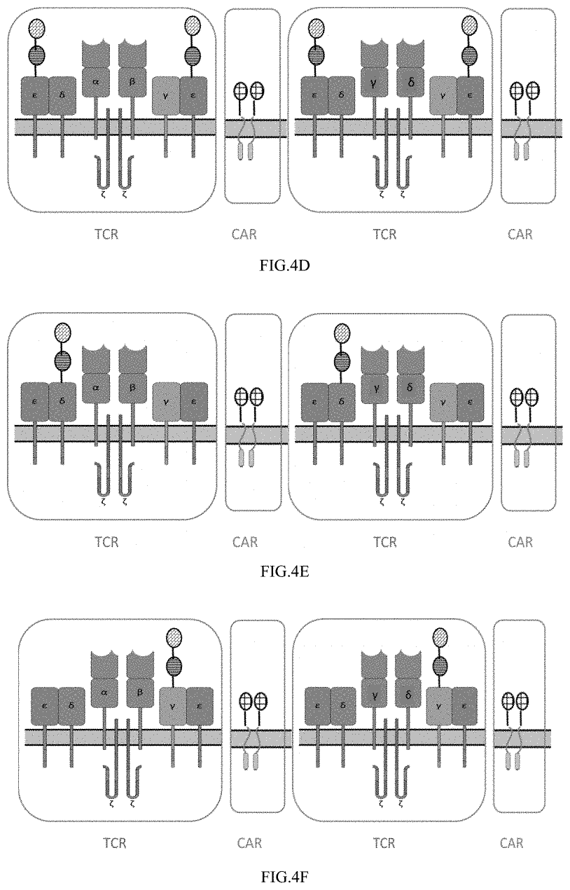

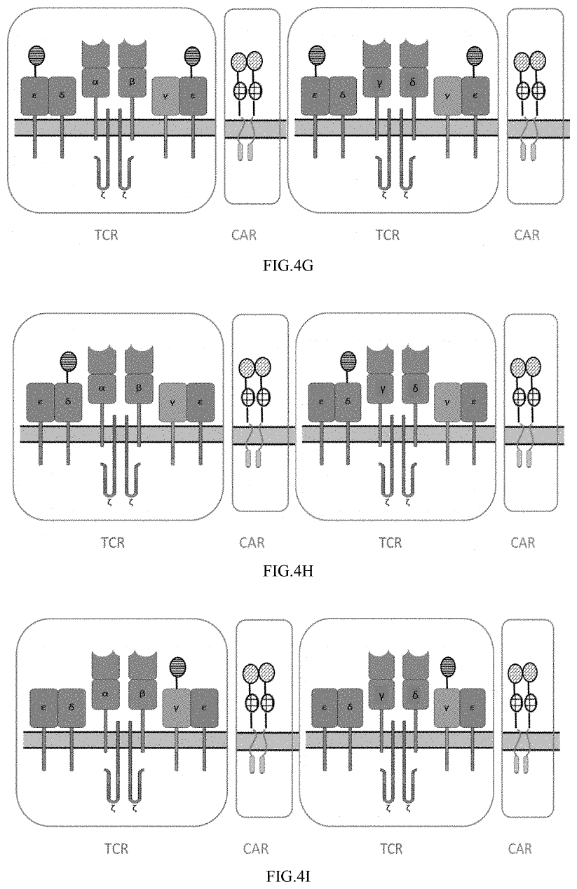

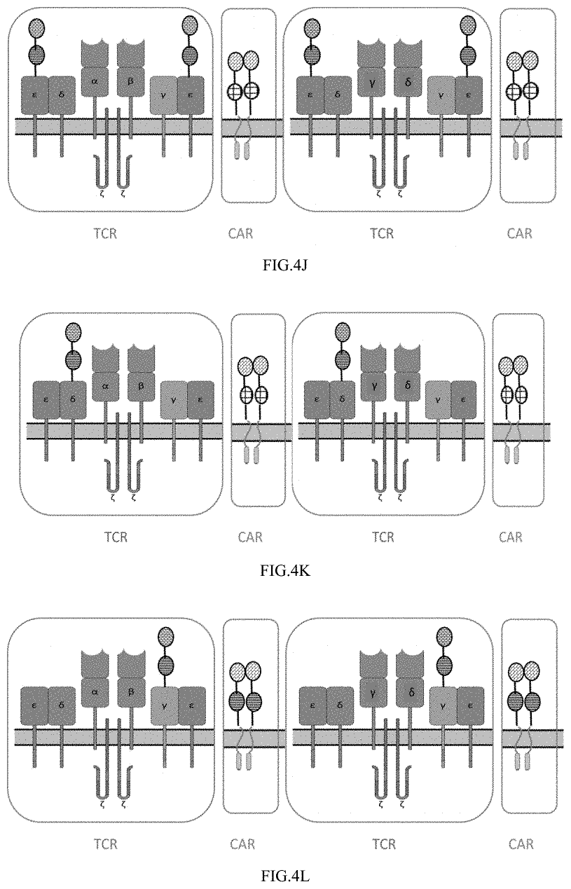

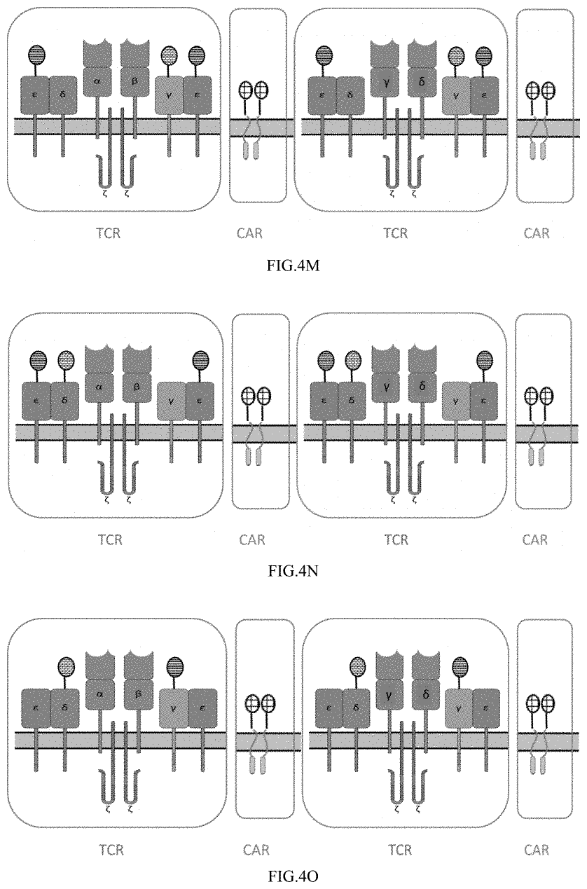

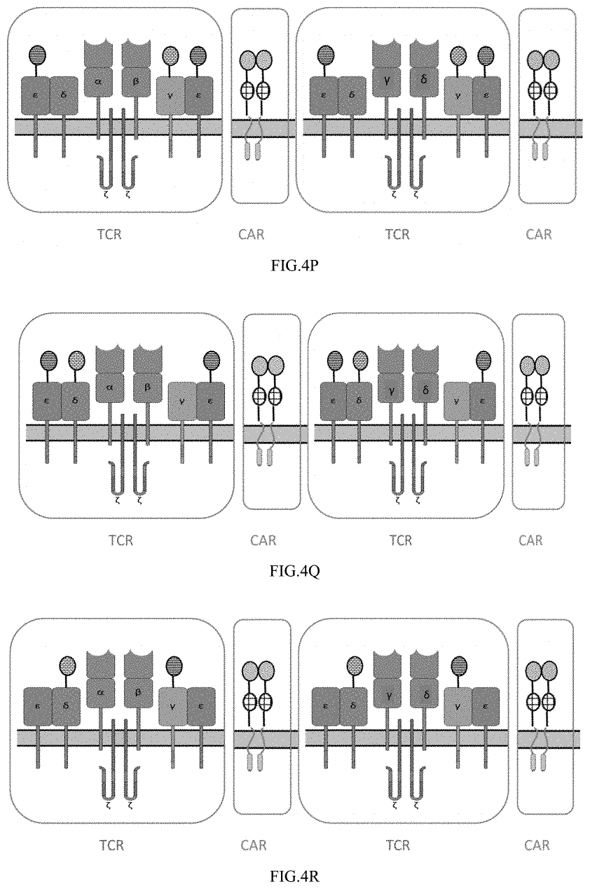

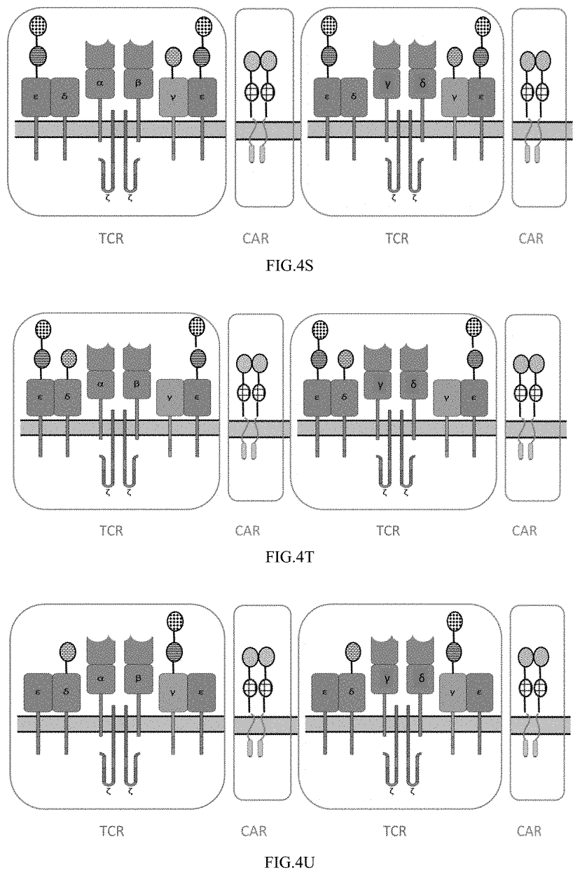

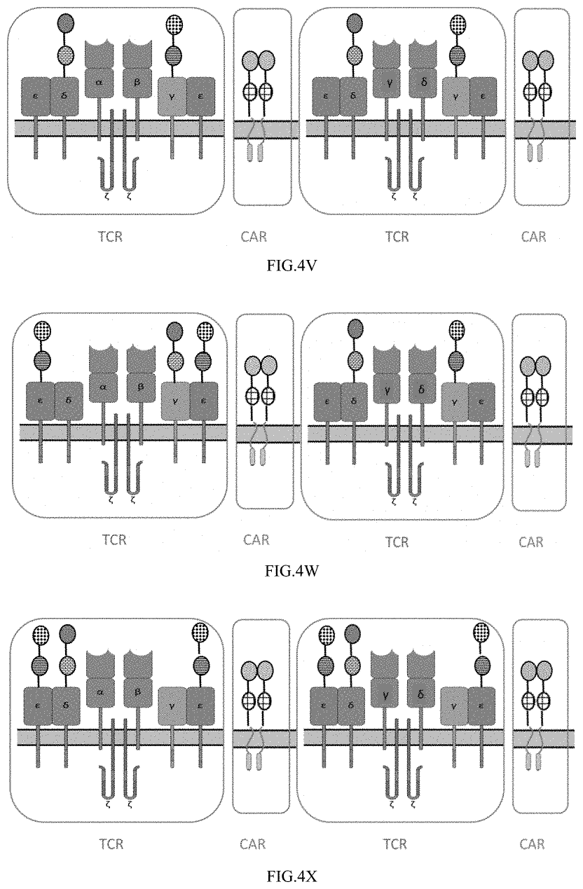

In some embodiments, a modified TCR complex of a subject system comprises an antigen binding domain fused to CD3-epsilon chain, FIG. 2A. In some embodiments, a modified TCR complex of a subject system comprises an antigen binding domain fused to a CD3-delta chain, FIG. 2B. In some embodiments, a modified TCR complex of a subject system comprises an antigen binding domain fused to a CD3-gamma chain, FIG. 2C. In some embodiments, a modified TCR complex of a subject system comprises an antigen binding domain fused to a TCR-alpha chain or a TCR-gamma chain, FIG. 2D. In some embodiments, a modified TCR complex of a subject system comprises an antigen binding domain fused to a TCR-beta chain or a TCR-delta chain, FIG. 2E. In some embodiments, a modified TCR complex of a subject system comprises an antigen binding domain fused to a TCR-gamma chain. In some embodiments, a modified TCR complex of a subject system comprises an antigen binding domain fused to a TCR-delta chain.

A modified TCR complex of a subject system can comprise more than one antigen binding domain, for example at least 2 antigen binding domains (e.g., at least 3, 4, 5, 6, 7, 8, 9, or 10 antigen binding domains). In some embodiments, a modified TCR complex of a subject system comprises at least two antigen binding domains. The at least two antigen binding domains can be the same antigen binding domain. For example, the two antigen binding domains may be identical molecules capable of binding to the same ligand. The at least two antigen binding domains can be different antigen binding domains. For example, the two antigen binding domains may be different molecules capable of binding to the same or different ligand. In some cases, a modified TCR comprises a third antigen binding domain linked to (i) the second antigen binding domain, (ii) at least one TCR chain selected from an alpha chain, a beta chain, a gamma chain and a delta chain of a T cell receptor (iii) an epsilon chain, delta chain, and/or a gamma chain of cluster of differentiation 3 (CD3), or (iv) CD3 zeta chain.

In some embodiments, a first antigen binding domain is fused to a first CD3-epsilon chain and a second antigen binding domain is fused to a second CD3-epsilon chain of a TCR complex, FIG. 2F. In some embodiments, a first antigen binding domain is fused to CD3-epsilon chain and a second antigen binding domain is fused to a CD3-gamma chain, FIG. 2G. In some embodiments, the first and second antigen binding domain are linked to the same chain. For example, a modified TCR complex of a subject system can comprise a first antigen binding domain fused to a second antigen binding domain which in turn in fused to CD3-epsilon chain, FIG. 2H. In some embodiments, a first antigen binding domain is fused to TCR-alpha chain and a second antigen binding domain is fused to a TCR-beta chain, or a first antigen binding domain is fused to TCR-gamma chain and a second antigen binding domain is fused to a TCR-delta chain. The first and the second antigen binding domains may be different antigen binding domains, as indicated by the black and black and white striped ovals (FIG. 2I). The first and the second antigen binding domains may be the same antigen binding domain, as indicated by the similarly shaded ovals (FIG. 2J).

In some embodiments, a modified TCR complex of a subject system comprises a first antigen binding domain fused to a second antigen binding domain which in turn in fused to a CD3-delta chain, FIG. 2K. In some embodiments, a modified TCR complex of a subject system comprises a first antigen binding domain fused to a second antigen binding domain which in turn in fused to a CD3-gamma chain, FIG. 2L. In some embodiments, a modified TCR complex of a subject system comprises a first antigen binding domain fused to a second antigen binding domain which in turn in fused to a TCR-alpha chain or a TCR-gamma chain, FIG. 2M. In some embodiments, a modified TCR complex of a subject system comprises a first antigen binding domain fused to a second antigen binding domain which in turn in fused to a TCR-beta chain or a TCR-delta chain, FIG. 2N. The first and the second antigen binding domains may be different antigen binding domains. The first and the second antigen binding domains may be the same antigen binding domain.

In some embodiments, a modified TCR complex of a subject system comprises a first antigen binding domain fused to CD3-epsilon chain and a second antigen binding domain fused to a CD3-delta chain, FIG. 2O. In some embodiments, a modified TCR complex of a subject system comprises a first antigen binding domain fused to a CD3-delta chain and a second antigen binding domain fused to a CD3-gamma chain, FIG. 2P. In some embodiments, a modified TCR complex of a subject system comprises a first antigen binding domain fused to a TCR-alpha chain or a TCR-gamma chain, and a second antigen binding domain fused to CD3-epsilon chain, FIG. 2Q. In some embodiments, a modified TCR complex of a subject system comprises a first antigen binding domain fused to a TCR-beta chain or a TCR-delta chain, and a second antigen binding domain fused to a CD3-epsilon chain, FIG. 2R. In some embodiments, a modified TCR complex of a subject system comprises a first antigen binding domain fused to a TCR-alpha chain or a TCR-gamma chain, and a second antigen binding domain fused to a CD3-gamma chain, FIG. 2S. In some embodiments, a modified TCR complex of a subject system comprises a first antigen binding domain fused to a TCR-beta chain or a TCR-delta chain, and a second antigen binding domain fused to a CD3-gamma chain, FIG. 2T. In some embodiments, a modified TCR complex of a subject system comprises a first antigen binding domain fused to a TCR-alpha chain or a TCR-gamma chain, and a second antigen binding domain fused to a CD3-delta chain, FIG. 2U. In some embodiments, a modified TCR complex of a subject system comprises a first antigen binding domain fused to a TCR-beta chain or a TCR-delta chain, and a second antigen binding domain fused to a delta chain, FIG. 2V.

In various embodiments of the aspects herein, a modified TCR complex comprises a TCR previously identified. In some cases, the TCR can be identified using whole-exomic sequencing. For example, a TCR can target a neoantigen or neoepitope that is identified by whole-exomic sequencing of a target cell. Alternatively, the TCR can be identified from autologous, allogenic, or xenogeneic repertoires. Autologous and allogeneic identification can entail a multistep process. In both autologous and allogeneic identification, dendritic cells (DCs) can be generated from CD14-selected monocytes and, after maturation, pulsed or transfected with a specific peptide. Peptide-pulsed DCs can be used to stimulate autologous or allogeneic immune cells, such as T cells. Single-cell peptide-specific T cell clones can be isolated from these peptide-pulsed T cell lines by limiting dilution. Subject TCRs of interest can be identified and isolated. Alpha, beta, gamma, and delta chains of a TCR of interest can be cloned, codon optimized, and encoded into a vector, for instance a lentiviral vector. In some embodiments, portions of the TCR can be replaced. For example, constant regions of a human TCR can be replaced with the corresponding murine regions. Replacement of human constant regions with corresponding murine regions can be performed to increase TCR stability. The TCR can also be identified with high or supraphysiologic avidity ex vivo. In some cases, a method of identifying a TCR can include immunizing transgenic mice that express the human leukocyte antigen (HLA) system with human tumor proteins to generate T cells expressing TCRs against human antigens (see e.g., Stanislawski et al., Circumventing tolerance to a human MDM2-derived tumor antigen by TCR gene transfer, Nature Immunology 2, 962-970 (2001)). An alternative approach can be allogeneic TCR gene transfer, in which tumor-specific T cells are isolated from a subject experiencing tumor remission and reactive TCR sequences can be transferred to T cells from another subject that shares the disease but may be non-responsive (de Witte, M. A., et al., Targeting self-antigens through allogeneic TCR gene transfer, Blood 108, 870-877(2006)). In some cases, in vitro technologies can be employed to alter a sequence of a TCR, enhancing their tumor-killing activity by increasing the strength of an interaction (avidity) of a weakly reactive tumor-specific TCR with target antigen (Schmid, D. A., et al., Evidence for a TCR affinity threshold delimiting maximal CD8 T cell function. J. Immunol. 184, 4936-4946 (2010)).

The antigen binding domain of a subject CAR and a modified TCR complex can bind to epitopes that are present on different antigens. In some cases, the antigen binding domains of the CAR and the modified TCR complex bind epitopes present on a common antigen. In some cases, the first antigen binding domain and the second antigen binding domain comprise the same amino acid sequence. In some cases, the first antigen binding domain and the second antigen binding domain comprise the different amino acid sequences.

In some cases, the first antigen binding domain of a subject CAR and a second antigen binding domain of modified TCR complex can bind to the same epitope, i.e. the first epitope and the second epitope are the same. In some cases, the first antigen binding domain of a subject CAR and a second antigen binding domain of modified TCR complex can bind to the different epitopes, i.e. the first epitope and the second epitope are the different.

The first epitope and/or the second epitope may be present on one or more cell surface antigens. The one or more cell surface antigens can be tyrosine kinase receptors, serine kinase receptors, histidine kinase receptor, G-protein coupled receptors (GPCR), and the like.

The first epitope and/or the second epitope may be present on an immune checkpoint receptor or immune checkpoint receptor ligand. In some embodiments, the immune checkpoint receptor or immune checkpoint receptor ligand can be PD-1, PD-L1, PD-L2, CTLA-4, TIM-3, LAG3, BLTA, TIGIT, CD47 or CD40.

The first epitope and/or the second epitope may be present on a cytokine or a cytokine receptor. A cytokine receptor can be, for example, CCR2b, CXCR2 (CXCL1 receptor), CCR4 (CCL17 receptor), Gro-a, IL-2, IL-7, IL-15, IL-21, IL-12, Heparinase, CD137L, LEM, Bcl-2, CCL17, CCL19 or CCL2.

The first epitope and/or the second epitope can be present on a tumor-associated antigen. The epitope may be, for instance a tumor epitope. A tumor-associated antigen can be selected from the group consisting of: 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, BCMA, b-Catenin, bcr-abl, bcr-abl p190 (e1a2), bcr-abl p210 (b2a2), bcr-abl p210 (b3a2), BING-4, CA-125, CAG-3, CAIX, CAMEL, Caspase-8, CD171, CD19, CD20, CD22, CD23, CD24, CD30, CD33, CD38, CD44v7/8, CD70, CD123, CD133, CDC27, CDK-4, CEA, CLCA2, CLL-1, CTAG1B, Cyp-B, DAM-10, DAM-6, DEK-CAN, DLL3, EGFR, EGFRvIII, EGP-2, EGP-40, ELF2, Ep-CAM, EphA2, EphA3, erb-B2, erb-B3, erb-B4, ES-ESO-1a, ETV6/AML, FAP, FBP, fetal acetylcholine receptor, FGF-5, FN, FR-α, G250, GAGE-1, GAGE-2, GAGE-3, GAGE-4, GAGE-5, GAGE-6, GAGE-7B, GAGE-8, GD2, GD3, GnT-V, Gp100, gp75, GPC3, GPC-2, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, L1-CAM, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, and κ-light chain. In some embodiments, a first epitope and/or a second epitope can be EGFR, EGFRvIII, GPC3, GPC-2, DLL3, BCMA, CD19, CD20, CD22, CD123, CLL-1, CD30, CD33, HER2, MSLN, PSMA, CEA, GD2, IL13Rα2, CAIX, L1-CAM, CA125, CD133, FAP, CTAG1B, MUC1, FR-α, CD70, CD171, ROR1, and any combination thereof. In some cases, a first epitope and/or a second epitope can be CLL-1, CD33, BCMA, CD19, and any combination thereof. In a preferred case, a first epitope and/or a second epitope can be CLL-1, CD33, and a combination thereof. In another preferred case, a first epitope and/or a second epitope can be BCMA, CD19, and a combination thereof.

The first epitope and/or the second epitope may be present on a neoantigen. The first epitope and/or the second epitope may be a neoepitope.

Neoantigens and neoepitopes generally refer to tumor-specific mutations that in some cases trigger an antitumor T cell response. For example, these endogenous mutations can be identified using a whole-exomic-sequencing approach. Tran E, et al., “Cancer immunotherapy based on mutation-specific CD4+ T cells in a patient with epithelial cancer,” Science 344: 641-644 (2014). An antigen binding domain, for example, that of a subject CAR or a modified TCR complex can exhibit specific binding to a tumor-specific neo-antigen. Neoantigens bound by antigen binding domains of a CAR or modified TCR complex can be expressed on a target cell, and for example, are neoantigens and neoepitopes encoded by mutations in any endogenous gene. In some cases, the first and/or second antigen binding domains bind a neoantigen or neoepitope encoded by a mutated gene. The gene can be selected from the group consisting of: ABL1, ACO1 1997, ACVR2A, AFP, AKT1, ALK, ALPPL2, ANAPC1, APC, ARID1A, AR, AR-v7, ASCL2, β2M, BRAF, BTK, C15ORF40, CDH1, CLDN6, CNOT1, CT45A5, CTAG1B, DCT, DKK4, EEF1B2, EEF1DP3, EGFR, EIF2B3, env, EPHB2, ERBB3, ESR1, ESRP1, FAM11 IB, FGFR3, FRG1B, GAGE1, GAGE 10, GATA3, GBP3, HER2, IDH1, JAK1, KIT, KRAS, LMAN1, MABEB 16, MAGEA1, MAGEA10, MAGEA4, MAGEA8, MAGEB 17, MAGEB4, MAGEC1, MEK, MLANA, MLL2, MMP13, MSH3, MSH6, MYC, NDUFC2, NRAS, NY-ESO, PAGE2, PAGE5, PDGFRa, PIK3CA, PMEL, pol protein, POLE, PTEN, RAC1, RBM27, RNF43, RPL22, RUNX1, SEC31A, SEC63, SF3B 1, SLC35F5, SLC45A2, SMAP1, SMAP1, SPOP, TFAM, TGFBR2, THAP5, TP53, TTK, TYR, UBR5, VHL, and XPOT.

In some embodiments, a first epitope and/or a second epitope which can be bound by the first and/or second antigen binding domain can be present on a stroma. Stroma generally refers to tissue which, among other things, provides connective and functional support of a biological cell, tissue, or organ. A stroma can be that of the tumor microenvironment. The first epitope and/or second epitope may be present on a stromal antigen. Such an antigen can be on the stroma of the tumor microenvironment. Neoantigens and neoepitopes, for example, can be present on tumor endothelial cells, tumor vasculature, tumor fibroblasts, tumor pericytes, tumor stroma, and/or tumor mesenchymal cells. Example antigens include, but are not limited to, CD34, MCSP, FAP, CD31, PCNA, CD117, CD40, MMP4, and Tenascin.

In some embodiments, a first epitope and/or a second epitope can be present on an antigen presented by a major histocompatibility complex (MHC). An MHC can be human leukocyte antigen (HLA) class I or class II. An HLA can be HLA-A, HLA-B, HLA-C, HLA-HLA-E, HLA-F, HLA-G, HLA-DP, HLA-DQ, HLA-DR, HLA-DM, or HLA-DO. In some embodiments, a first epitope/and or a second epitope can be present on HLA-A*01, HLA-A*02, HLA-A*03, HLA-A*11, HLA-A*23, HLA-A*24, HLA-A*25, HLA-A*26, HLA-A*29, HLA-A*30, HLA-A*31, HLA-A*32, HLA-A*33, or HLA-A*24, HLA-B*27, HLA-B*35, HLA-B*48, HLA-B*55, and the like.

In some embodiments, a first epitope and/or a second epitope can be soluble (e.g., not bound to a cell). In some cases, the antigen can be soluble, e.g., a soluble antigen. The first epitope and/or the second epitope may be present on a universal antigen. In some cases, the antigen binding domain of a subject CAR and/or a modified TCR complex each can bind to multiple epitopes, e.g., multiple specificities.

Binding of the first antigen binding domain to the first epitope or binding of the second antigen binding domain to the second epitope can activate an immune cell activity of an immune cell expressing the system. In some cases, binding of the first antigen binding domain to the first epitope and binding of the second antigen binding domain to the second epitope activates an immune cell activity of an immune cell expressing the system. In some embodiments, a system for inducing activity of an immune cell and/or a target cell can comprise more than two antigen binding domains. For example, a system can comprise a first, second, third, fourth, fifth, sixth, seventh, eighth, ninth, or tenth or even more antigen binding domains. In some embodiments, binding of the third antigen binding domain to a third epitope activates an immune cell activity of an immune cell expressing the system. In some embodiments, binding of the first antigen binding domain to the first epitope, binding of the second antigen binding domain to the second epitope, and binding of the third antigen binding domain to the third epitope activates an immune cell activity of an immune cell expressing the system. Any number of antigen binding domains can be used in systems of the present disclosure, and the number of antigen binding domains is not limited to one, two or three.

In some embodiment, two or more antigen binding domains are linked to, optionally in tandem, (i) at least one TCR chain selected from an alpha chain, a beta chain, a gamma chain and a delta chain of a T cell receptor (ii) an epsilon chain, a delta chain, and/or a gamma chain of cluster of differentiation 3 (CD3), (iii) a CD3 zeta chain, and wherein binding of the two or more antigen binding domains to their respective epitopes activates an immune cell activity of an immune cell expressing the system. Where desired, the two or more antigen binding domains are linked to separate chains of the TCR complex. Alternatively, the two or more antigen binding domains are linked to one chain of the TCR complex. In some embodiments, the two or more antigen binding domains are linked in tandem on the epsilon chain, the delta chain, and/or the gamma chain of cluster of differentiation 3 (CD3).

The immune cell activity that is activated in the immune cell expressing the system can be any of a variety of cellular activities. In some embodiments, the immune cell activity is selected from the group consisting of clonal expansion of the immune cell; cytokine release by the immune cell; cytotoxicity of the immune cell; proliferation of the immune cell; differentiation, dedifferentiation or transdifferentiation of the immune cell; movement and/or trafficking of the immune cell; exhaustion and/or reactivation of the immune cell; and release of other intercellular molecules, metabolites, chemical compounds, or combinations thereof by the immune cell.

In some embodiments, the immune cell activity comprises clonal expansion of the immune cell. Clonal expansion can comprise the generation of daughter cells arising from the immune cell. In a clonal expansion, progeny of the immune cell can comprise a system provided herein. In a clonal expansion, progeny of the immune cell can comprise a CAR provided herein. In a clonal expansion, progeny of the immune cell can comprise a modified TCR complex provided herein. In a clonal expansion, progeny of the immune cell can comprise the CAR and the TCR provided herein. Clonal expansion of an immune cell comprising a system provided herein can be greater than that of a comparable immune cell lacking the system, a comparable immune cell lacking one or more components of the system (e.g., CAR, modified TCR complex), and/or a comparable immune cell in which only one of the first and second antigen binding domains are bound to their respective epitopes. Clonal expansion of an immune cell comprising a system provided herein can be about 5 fold to about 10 fold, about 10 fold to about 20 fold, about 20 fold to about 30 fold, about 30 fold to about 40 fold, about 40 fold to about 50 fold, about 50 fold to about 60 fold, about 60 fold to about 70 fold, about 70 fold to about 80 fold, about 80 fold to about 90 fold, about 90 fold to about 100 fold, about 100 fold to about 200 fold, about 200 fold to about 300 fold, about 300 fold to about 400 fold, about 400 fold to about 500 fold, about 500 fold to about 600 fold, about 600 fold to about 700 fold greater than a comparable immune cell lacking the system, a comparable immune cell lacking one or more components of the system (e.g., CAR, modified TCR complex), and/or a comparable immune cell in which only one of the first and second antigen binding domains are bound to their respective epitopes. In some embodiments, clonal expansion can comprise quantifying the number of immune cells. Quantifying a number of immune cells can comprise, flow cytometry, Trypan Blue exclusion, and/or hemocytometry.

In some embodiments, the immune cell activity comprises cytokine release by the immune cell. In some embodiments, the immune cell activity comprises release of intercellular molecules, metabolites, chemical compounds or combinations thereof. Cytokine release by the immune cell can comprise the release of IL-1, IL-2, IL-4, IL-5, IL-6, IL-13, IL-17, IL-21, IL-22, IFNγ, TNFα, CSF, TGFβ, granzyme, and the like. In some embodiments, cytokine release may be quantified using ELISA, flow cytometry, western blot, and the like. Cytokine release by an immune cell comprising a system provided herein can be greater than that of a comparable immune cell lacking the system, a comparable immune cell lacking one or more components of the system (e.g., CAR, modified TCR complex), and/or a comparable immune cell in which only one of the first and second antigen binding domains are bound to their respective epitopes. An immune cell comprising a system provided herein can generate from about 1 fold, 2 fold, 3 fold, 4 fold, 5 fold, 6 fold, 7 fold, 8 fold, 9 fold, 10 fold, 11 fold, 12 fold, 13 fold, 14 fold, 15 fold, 20 fold, 30 fold, 40 fold, 50 fold, 60 fold, 70 fold, 80 fold, 90 fold, 100 fold, 150 fold, 200 fold, 250 fold, or over 300 fold greater cytokine release as compared to a comparable immune cell lacking the system, a comparable immune cell lacking one or more components of the system (e.g., CAR, modified TCR complex), and/or a comparable immune cell in which only one of the first and second antigen binding domains are bound to their respective epitopes. In some embodiments, cytokine release can be quantified, in vitro or in vivo.

In some embodiments, the immune cell activity comprises cytotoxicity of the immune cell. In some examples, the systems and compositions of the present disclosure, when expressed in an immune cell, can be used for killing a target cell. An immune cell or population of immune cells expressing a subject system can induce death of a target cell. Killing of a target cell can be useful for a variety of applications, including, but not limited to, treating a disease or disorder in which a cell population is desired to be eliminated or its proliferation desired to be inhibited. Cytotoxicity can refer to the killing of the target cell. Cytotoxicity can also refer to the release of cytotoxic cytokines, for example IFNγ, TNF alpha, TNF beta, GM-CSF, or granzyme, by the immune cell. In some cases, a subject system expressed in immune cells can alter the (i) release of cytotoxins such as perforin, granzymes, and granulysin and/or (ii) induction of apoptosis via Fas-Fas ligand interaction between the T cells and target cells, thereby triggering the destruction of target cells. In some embodiments, cytotoxicity can be quantified by a cytotoxicity assay including, a co-culture assay, ELISPOT, chromium release cytotoxicity assay, and the like. Cytotoxicity of an immune cell comprising a system provided herein can be greater than that of a comparable immune cell lacking the system, a comparable immune cell lacking one or more components of the system (e.g., CAR, modified TCR complex), and/or a comparable immune cell in which only one of the first and second antigen binding domains are bound to their respective epitopes. An immune cell comprising a system provided herein can be about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200% more cytotoxic to target cells as compared to a comparable immune cell lacking the system, a comparable immune cell lacking one or more components of the system (e.g., CAR, modified TCR complex), and/or a comparable immune cell in which only one of the first and second antigen binding domains are bound to their respective epitopes. An immune cell comprising a system provided herein can induce death of target cells that is at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200% greater than that of a comparable immune cell lacking the system, a comparable immune cell lacking one or more components of the system (e.g., CAR, modified TCR complex), and/or a comparable immune cell in which only one of the first and second antigen binding domains are bound to their respective epitopes. Similarly, an immune cell comprising a system provided herein can induce death of target cells that is at least 1 fold, 2 fold, 3 fold, 4 fold, 5 fold, 6 fold, 7 fold, 8 fold, 9 fold, 10 fold, 15 fold, 20 fold, 40 fold, 60 fold, 80 fold, or 100 fold greater than that of a comparable immune cell lacking the system, a comparable immune cell lacking one or more components of the system (e.g., CAR, modified TCR complex), and/or a comparable immune cell in which only one of the first and second antigen binding domains are bound to their respective epitopes. In some embodiments, an immune cell expressing a subject system can induce apoptosis in target cells displaying target epitopes on their surface.

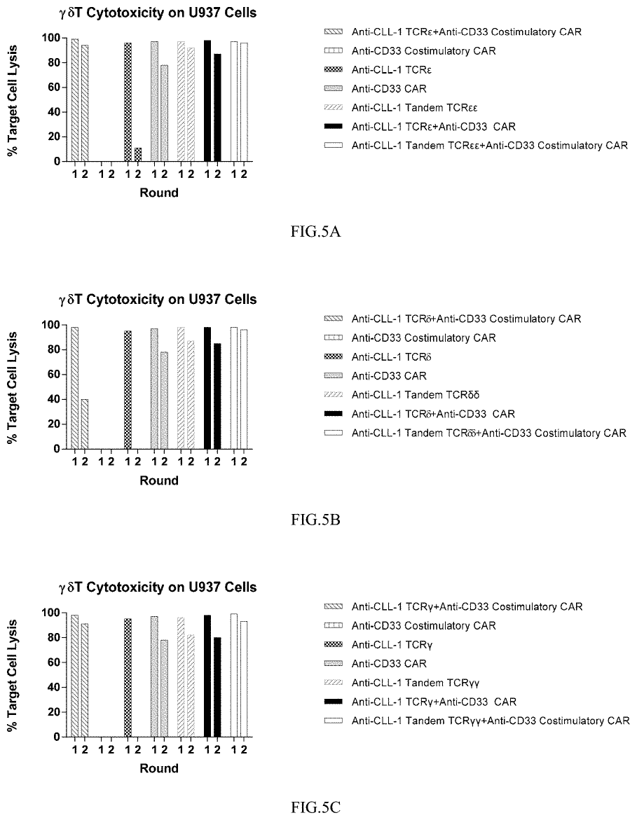

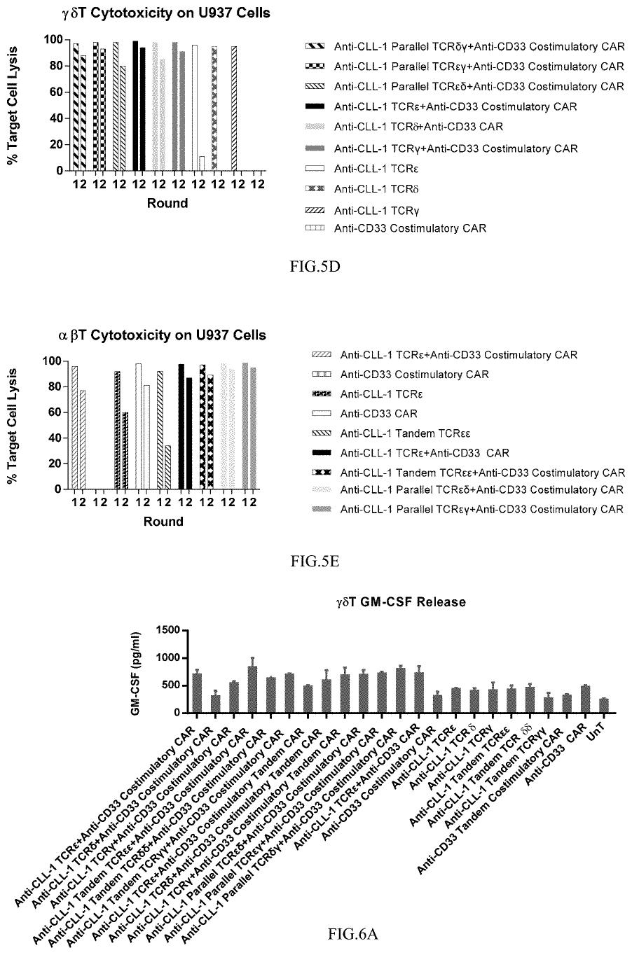

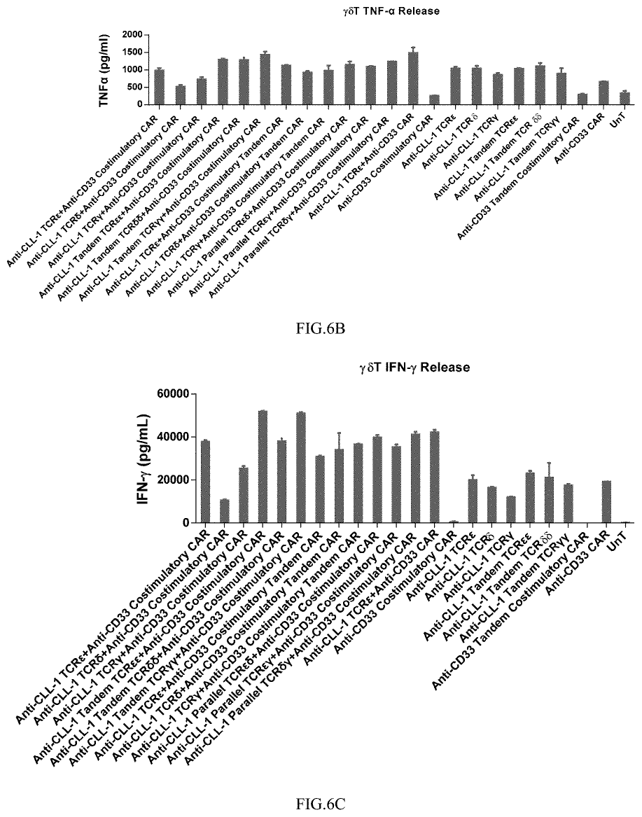

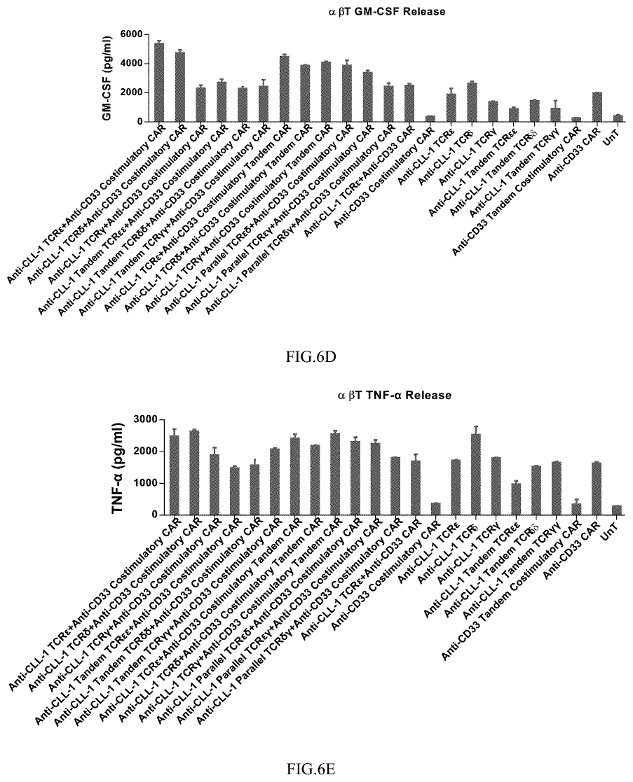

In some embodiments, administration of a subject immune cell comprising a subject system can generate a side effect in a subject in need thereof. A toxicity can comprise cytokine release storm, tumor lysis syndrome, on-target off-tumor toxicity, and combinations thereof. For example, in some cases, a potential toxicity level for an alpha beta T cell can be greater than that of a gamma delta T cell in the case where both cells express the same subject system, FIG. 6A and FIG. 6D. In some embodiments, a method can comprise administering a population of cells that comprise a gamma delta T cell that comprises a subject system to reduce potential side effects in a subject. In some aspects, the use of subject systems in gamma delta T cells can reduce potential side effects in a subject by at least about 1 fold, 2 fold, 3 fold, 4 fold, 5 fold, 6 fold, 7 fold, 8 fold, 9 fold, 10 fold, 11 fold, 12 fold, 15 fold, 20 fold, 30 fold, 40 fold, 50 fold, 75 fold, 100 fold, 150 fold, 200 fold, or up to about 500 fold. In some embodiments, pharmaceutical compositions can be formulated with gamma delta T cells that comprise a subject system.

In an aspect, a toxicity can comprise production of cytokines in response to contact with a non-target cell and/or killing of the non-target cell. Non-target cells can comprise: non-diseased cells, non-cancerous cells, endogenous cell, or a cell that expresses a subject target antigen but is not cancerous or diseased (for example, on-target, off-tumor toxicity). Toxicity associated with administering subject immune cells expressing subject systems can be determined in vitro or in vivo. Methods of evaluating toxicities can comprise performing an in vitro and/or in vivo assay provided herein, such as ELISA and/or CFU assays and the like.

An ELISA assay can be used to identify and/or quantify cytokine production by subject immune cells comprising subject systems. In an aspect, an ELISA for evaluating a toxicity can detect any one of: tumor necrosis factor-alpha (TNF-α), interferon γ (IFN-γ), interleukin 6 (IL-6), and IL-10. In some cases, a colony forming unit (CFU) assay can be performed to evaluate a toxicity associated with a cell expressing a subject system against a number of non-diseased targets, for example normal hematopoietic stem cells.

In comparative assays of subject systems, a first subject system can have a greater or reduced toxicity level as compared to a second subject system. Depending on the application of immune cells comprising subject systems, a more or less potent immune response may be warranted and may factor into deciding what system to express in a subject immune cell. In an aspect, an immune cell expressing a subject system that does not comprise a CD3zeta chain may have reduced toxicity as compared to a comparable immune cell comprising a subject system comprising the CD3 zeta chain. Similarly, a subject system that does not comprise a co-stimulatory domain may have reduced toxicity as compared to a comparable immune cell comprising a subject system with a co-stimulatory domain. In an aspect, the reduction of the toxicity can be from about 1 fold, 2 fold, 3 fold, 4 fold, 5 fold, 8 fold, or 10 fold less.

Another consideration can be the choice of immune cell in which to express a subject system. Different immune cells comprising the same subject system can perform differently in terms of cytokine production and cytotoxicity and can thereby have different levels of related toxicity independent of the subject system. For example, in some cases, gamma delta T cells expressing a subject system may produce more or less cytokines when contacted with a target as compared to a different immune cell expressing the same subject system. Alternatively, an αβ T cell expressing a subject system may produce more or less cytokines when contacted with a target as compared to a different immune cell expressing the same subject system. In an aspect, gamma delta T cells expressing a subject system may produce less GM-CSF and TNF alpha as compared to an alpha beta T cell expressing the same subject system. In another aspect, gamma delta T cells expressing a subject system may produce comparable amounts of IFNγ as compared to alpha beta T cell expressing the same subject system. This may indicate that in some cases, administering gamma delta T cells expressing a subject system may result in a more favorable cytokine profile associated with reduced systemic toxicity. A more favorable cytokine profile may refer to generating less cytokines associated with a cytokine storm or systemic toxicity.

In an embodiment, an immune cell comprising a subject system may also be tested for any toxicity and/or functionality associated with repeated antigen stimulation. Repeated antigen stimulation may mimic conditions in the tumor milieu that a subject immune cell comprising a subject system may encounter. Any one of the subject antigens can be utilized to perform a repeated antigen stimulation. In an aspect, an immune cell comprising a subject system may undergo at least 2, 3, 4, 5, 6, 7, 8, 9, or 10 rounds of antigen stimulation. Stimulation can occur in vitro or in vivo and can last any about of time. In an aspect, a stimulation lasts 30 minutes, 1 hour, 3 hours, 8 hours, 10 hours, 24 hours, 48 hours, 72 hours, or up to about 100 hours. As previously described, the choice of immune cell expressing a subject system and/or the subject system can contribute to a level of toxicity, if any, and/or functionality associated with repeated antigen stimulation. In an aspect, a targeting domain of a system fused to the epsilon or gamma subunit of a CD3 within a TCR complex can have increased anti-tumor cytotoxicity as compared to a comparable immune cell expressing a comparable targeting domain fused to a delta subunit. Similarly, an immune cell that comprises a subject system that comprises a co-stimulatory domain and no CD3 zeta chain can have improved activity and/or reduced toxicity with repeated antigen stimulation as compared to a comparable immune cell expressing a comparable system. In some embodiments, such comparable system may be a comparable TCR only system or a comparable CAR system with an intracellular signaling domain devoid of a signaling domain of CD3 zeta. Additionally, in some cases, a subject system having a tandem or parallel design can have improved immune cell activity and/or reduced toxicity as compared to a comparable system without the tandem or parallel design. Suitable tandem or parallel designed are provided throughout herein with exemplary designs described in Example 1.

In some embodiments, cytotoxicity can be determined in vitro or in vivo. In some embodiments, determining cytotoxicity can comprise determining a level of disease after administration of cells comprising a system provided herein as compared to a level of disease prior to the administration. In some embodiments, determining cytotoxicity can comprise determining a level of disease after administration of cells comprising a system provided herein and a level of disease after administration of comparable immune cells lacking the system, comparable immune cells lacking one or more components of the system (e.g., CAR, modified TCR complex), and/or comparable immune cells in which only one of the first and second antigen binding domains are bound to their respective epitopes. In some embodiments, a level of disease on a target lesion can be measured as a Complete Response (CR); Disappearance of target lesions, Partial Response (PR); at least a 30% decrease in the sum of the longest diameter (LD) of target lesions taking as reference the baseline sum LD, Progression (PD); at least a 20% increase in the sum of LD of target lesions taking as reference the smallest sum LD recorded since the treatment started or the appearance of one or more new lesions, Stable Disease (SD); or, neither sufficient shrinkage to qualify for PR nor sufficient increase to qualify for PD taking as references the smallest sum LD. In some embodiments, a non-target lesion can be measured. A level of disease of a non-target lesion can be Complete Response (CR); disappearance of all non-target lesions and normalization of tumor marker level, Non-Complete Response; persistence of one or more non-target lesions, Progression (PD); or appearance of one or more new lesions.

In some embodiments, immune cell activity is proliferation of the immune cell. Proliferation of the immune cell can refer to expansion of the immune cell. Proliferation of the immune cell can refer to phenotypic changes of the immune cell. Proliferation of an immune cell comprising a system provided herein can be greater than that of a comparable immune cell lacking the system, a comparable immune cell lacking one or more components of the system (e.g., CAR, modified TCR complex), and/or a comparable immune cell in which only one of the first and second antigen binding domains are bound to their respective epitopes. Proliferation of an immune cell comprising a system provided herein can be about 5 fold to about 10 fold, about 10 fold to about 20 fold, about 20 fold to about 30 fold, about 30 fold to about 40 fold, about 40 fold to about 50 fold, about 50 fold to about 60 fold, about 60 fold to about 70 fold, about 70 fold to about 80 fold, about 80 fold to about 90 fold, about 90 fold to about 100 fold, about 100 fold to about 200 fold, from about 200 fold to about 300 fold, from about 300 fold to about 400 fold, from about 400 fold to about 500 fold, from about 500 fold to about 600 fold, from about 600 fold to about 700 fold greater than the proliferation of a comparable immune cell lacking the system provided herein, a comparable immune cell lacking one or more components of the system (e.g., CAR, modified TCR complex), and/or a comparable immune cell in which only one of the first and second antigen binding domains are bound to their respective epitopes. In some embodiments, proliferation can comprise quantifying the number of immune cells. Quantifying a number of immune cells can comprise flow cytometry, Trypan Blue exclusion, and/or hemocytometry. Proliferation can also be determined by phenotypic analysis of the immune cells. For example, clumping of immune cells in culture can signify proliferation of immune cells as compared to comparable immune cells lacking the system.

In some embodiments, immune cell activity can be differentiation, dedifferentiation, or transdifferentiation. Differentiation, dedifferentiation, or transdifferentation of an immune cell can be determined by evaluating phenotypic expression of markers of differentiation, dedifferentiation, or transdifferentation on a cell surface by flow cytometry. Differentiation, dedifferentiation, or transdifferentation of an immune cell can also be determined via CFU assay. In some embodiments, an immune cell comprising a system provided herein has increased differentiation ability as compared to a comparable immune cell lacking the system, a comparable immune cell lacking one or more components of the system (e.g., CAR, modified TCR complex), and/or a comparable immune cell in which only one of the first and second antigen binding domains are bound to their respective epitopes. In some embodiments, an immune cell comprising a system provided herein has increased dedifferentiation ability as compared to a comparable immune cell lacking the system, a comparable immune cell lacking one or more components of the system (e.g., CAR, modified TCR complex), and/or a comparable immune cell in which only one of the first and second antigen binding domains are bound to their respective epitopes. In some embodiments, an immune cell comprising a system provided herein has greater transdifferentiation ability as compared to a comparable immune cell lacking the system, a comparable immune cell lacking one or more components of the system (e.g., CAR, modified TCR complex), and/or a comparable immune cell in which only one of the first and second antigen binding domains are bound to their respective epitopes.

In some embodiments, immune cell activity can be movement and/or trafficking of the immune cell comprising the system. In some embodiments, movement can be determined by quantifying localization of the immune cell to a target site. For example, immune cells comprising a subject system can be quantified at a target site after administration, for example at a site that is not the target site. Quantification can be performed by isolating a lesion and quantifying a number of immune cells, for example tumor infiltrating lymphocytes, comprising the system. Movement and/or trafficking of an immune cell comprising a system provided herein can be greater than that of a comparable immune cell lacking the system, a comparable immune cell lacking one or more components of the system (e.g., CAR, modified TCR complex), and/or a comparable immune cell in which only one of the first and second antigen binding domains are bound to their respective epitopes In some embodiments, the number of immune cells comprising the system at a target site, for example a tumor lesion, can be about 5×, 10×, 15×, 20×, 25×, 30×, 35×, or 40× that of the number of comparable immune cells lacking the system, comparable immune cells lacking one or more components of the system (e.g., CAR, modified TCR complex), and/or comparable immune cells in which only one of the first and second antigen binding domains are bound to their respective epitopes. Trafficking can also be determined in vitro utilizing a transwell migration assay. In some embodiments, the number of immune cells comprising the system at a target site, for example in a transwell migration assay, can be about 5×, 10×, 15×, 20×, 25×, 30×, 35×, or 40× that of the number of comparable immune cells lacking the system, comparable immune cells lacking one or more components of the system (e.g., CAR, modified TCR complex), and/or comparable immune cells in which only one of the first and second antigen binding domains are bound to their respective epitopes.

In some embodiments, immune cell activity can be exhaustion and/or activation of the immune cell. Exhaustion and/or activation of an immune cell can be determined by phenotypic analysis by flow cytometry or microscopic analysis. For example, expression levels of markers of exhaustion, for instance programmed cell death protein 1 (PD1), lymphocyte activation gene 3 protein (LAG3), 2B4, CD160, Tim3, and T cell immunoreceptor with immunoglobulin and ITIM domains (TIGIT), can be determined quantitatively and/or qualitatively. In some cases, immune cells, such as T cells, can lose effector functions in a hierarchical manner and become exhausted. As a result of exhaustion, functions such as IL-2 production and cytokine expression, as well as high proliferative capacity, can be lost. Exhaustion can also be followed by defects in the production of IFNγ, TNF and chemokines, as well as in degranulation. Exhaustion or activation of an immune cell comprising a system provided herein can be greater than that of a comparable immune cell lacking the system, a comparable immune cell lacking one or more components of the system (e.g., CAR, modified TCR complex), and/or a comparable immune cell in which only one of the first and second antigen binding domains are bound to their respective epitopes. In some embodiments, the immune cell comprising the system provided herein can undergo at least about a 1 fold, 2 fold, 3 fold, 4 fold, 5 fold, 6 fold, 7 fold, 8 fold, 9 fold, 10 fold, 11 fold, 12 fold, 13 fold, 14 fold, 15 fold, 20 fold, 30 fold, 40 fold, 50 fold, 60 fold, 70 fold, 80 fold, 90 fold, 100 fold, 150 fold, 200 fold, 250 fold, or over 300 fold increase in exhaustion or activation as compared to a comparable immune cell lacking the system, a comparable immune cell lacking one or more components of the system (e.g., CAR, modified TCR complex), and/or a comparable immune cell in which only one of the first and second antigen binding domains are bound to their respective epitopes. In some embodiments, the immune cell comprising the system provided herein can undergo at least about a 1 fold, 2 fold, 3 fold, 4 fold, 5 fold, 6 fold, 7 fold, 8 fold, 9 fold, 10 fold, 11 fold, 12 fold, 13 fold, 14 fold, 15 fold, 20 fold, 30 fold, 40 fold, 50 fold, 60 fold, 70 fold, 80 fold, 90 fold, 100 fold, 150 fold, 200 fold, 250 fold, or over 300 fold decrease in exhaustion or activation as compared to a comparable immune cell lacking the system, a comparable immune cell lacking one or more components of the system (e.g., CAR, modified TCR complex), and/or a comparable immune cell in which only one of the first and second antigen binding domains are bound to their respective epitopes.

In some embodiments, binding of the first antigen binding domain to the first epitope and binding of the second antigen domain to the second epitope activates cytotoxicity of a subject immune cell expressing the system. Cytotoxicity can be enhanced as compared to (i) binding of the first antigen binding domain to the first epitope alone, or (ii) binding of the second antigen binding domain to the second epitope alone. Cytotoxicity can be enhanced, as measured by percent killing in a cytotoxicity assay, as compared to (i) binding of the first antigen binding domain to the first epitope alone, or (ii) binding of the second antigen binding domain to the second epitope alone. A percent killing can be from about 5%, 10%, 15%, 20%, 25%, 30%, 35%, 40%, 45%, 50%, 55%, 60%, 65%, 70%, 75%, 80%, 85%, 90%, 95%, or up to about 100% of target cells after contacting as compared to (i) binding of the first antigen binding domain to the first epitope alone, or (ii) binding of the second antigen binding domain to the second epitope alone.

In some embodiments, binding of the first antigen binding domain to the first epitope and binding of the second antigen binding domain to the second epitope activates cytotoxicity of an immune cell expressing the system and reduces a side effect associated with the cytotoxicity as compared to (i) binding of the first antigen binding domain to the first epitope alone, or (ii) binding of the second antigen binding domain to the second epitope alone. In some embodiments, the side effect associated with the cytotoxicity is cytokine release syndrome. A reduction of a side effect, such as a decrease in cytokine release syndrome, can be from about 5%, 10%, 15%, 20%, 25%, 30%, 35%, 40%, 45%, 50%, 55%, 60%, 65%, 70%, 75%, 80%, 85%, 90%, 95%, or up to about 100% reduction as compared to (i) binding of the first antigen binding domain to the first epitope alone, or (ii) binding of the second antigen binding domain to the second epitope alone.

In some embodiments, binding of the first antigen binding domain to the first epitope and binding of the second antigen binding domain to the second epitope activates cytotoxicity of an immune cell expressing the system and increases persistence of cytotoxicity as compared to binding of the first antigen binding domain to the first epitope alone, or binding of the second antigen binding domain to the second epitope alone. Binding of the first antigen binding domain to the first epitope and binding of the second antigen binding domain to the second epitope can activate cytotoxicity of an immune cell expressing the system and increases persistence of said cytotoxicity as compared to binding of the first antigen binding domain to the first epitope alone, or binding of the second antigen binding domain to the second epitope alone when said system is expressed in an immune cell in a subject. An increase in persistence can be determined by quantifying a level of immune cells comprising the system after an administration. An increase in persistence can refer to the presence of immune cells comprising a system provided herein from 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 25 days, 30 days, 35 days, 40 days, 2 months, 3 months, 4 months, 5 months, 6 months, 7 months, 8 months, 9 months, 10 months, 1 year or more after administering as compared to comparable immune cells lacking the system, comparable immune cells lacking one or more components of the system (e.g., CAR, modified TCR complex), and/or a comparable immune cell in which only one of the first and second antigen binding domains are bound to their respective epitopes.

In an aspect, the present disclosure provides an isolated host cell expressing any system of the various embodiments herein (e.g., CAR, modified TCR complex). The isolated host cell can comprise a population of host cells. A host cell can be any suitable cell for expressing a subject system. In some cases, the host cell is an immune cell. The immune cell can be a lymphocyte such as a T cell. Non-limiting examples of T cells include CD8+ T cells, CD4+ T cells, αβ T cells, γδ T cells, Vγ9δ2 T cells, Vδ1 T cells, Vδ3 T cells and Vδ5 T cells. In some cases, the lymphocyte expressing a subject system is a natural killer (NK) cell, effector T cells, memory T cells, cytotoxic T cells, and/or T helper cells. In some cases, the lymphocyte expressing a subject system is a KHYG cell such as KHYG-1 cell or a derivative thereof.

In an aspect, the present disclosure provides an antigen-specific immune cell comprising at least two antigen binding domains, one of which is linked to a T cell receptor (TCR) complex and another that is linked to a chimeric antigen receptor (CAR). The antigen-specific immune cell can bind specifically to a target cell expressing one or more antigens recognized by the at least two antigen binding domains. The immune cell can be a lymphocyte such as a T cell. Non-limiting examples of T cells include CD8+ T cells, CD4+ T cells, αβ T cells, γδ T cells, Vγ9δ2 T cells, Vδ1 T cells, Vδ3 T cells and Vδ5 T cells. In some cases, the lymphocyte expressing a subject system is a natural killer (NK) cell, effector T cells, memory T cells, cytotoxic T cells, and/or T helper cells. In some cases, the lymphocyte expressing a subject system is a KHYG cell such as KHYG-1 cell or a derivative thereof.

In an aspect, the present disclosure provides a population of immune cells, individual immune cells expressing any system of the various embodiments herein, and wherein the population of immune cells is characterized in that: upon exposing the population of immune cells to a target cell population in a subject, the population of immune cells induces death of the target cells. The population of immune cells can induce death of at least 5%, 10%, 15%, 20%, 25%, 30%, 35%, 40%, 45%, 50%, 55%, 60%, 65%, 70%, 75%, 80%, 85%, 90%, 95%, or up to about 100% of the target cells and within about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 25 days, 30 days, 35 days, 40 days, 2 months, 3 months, 4 months, 5 months, 6 months, 7 months, 8 months, 9 months, 10 months, 1 year or more after the exposing.

The population of immune cell can comprise any of a variety of immune cells. In some cases, the population of immune cells comprises lymphocytes. The lymphocytes can be T cells. Non-limiting examples of T cells include CD8+ T cells, CD4+ T cells, αβ T cells, γδ T cells, Vγ9δ2 T cells, Vδ1 T cells, Vδ3 T cells and Vδ5 T cells. In some cases, the lymphocyte expressing a subject system is a natural killer (NK) cell, effector T cells, memory T cells, cytotoxic T cells, and/or T helper cells. In some cases, the lymphocyte expressing a subject system is a KHYG cell such as KHYG-1 cell or a derivative thereof.

The population of immune cells can comprise any suitable number of cells. The number of immune cells can be determined as the number of cells used in an in vitro assay. The number of immune cells can be determined as the number of cells administered to a subject. The number of immune cells can be determined as the number of cells prior to activation of any immune cell activity, such as proliferation and/or expansion. The population of immune cells can comprise at least about 1×10⁶ cells, at least about 2×10⁶ cells, at least about 3×10⁶ cells, at least about 4×10⁶ cells, at least about 5×10⁶ cells, at least about 6×10⁶ cells, at least about 7×10⁶ cells, at least about 8×10⁶ cells, at least about 9×10⁶ cells, 1×10⁷ cells, at least about 2×10⁷ cells, at least about 3×10⁷ cells, at least about 4×10⁷ cells, at least about 5×10⁷ cells, at least about 6×10⁷ cells, at least about 7×10⁷ cells, at least about 8×10⁷ cells, at least about 9×10⁷ cells, at least about 1×10⁸ cells, at least about 2×10⁸ cells, at least about 3×10⁸ cells, at least about 4×10⁸ cells, at least about 5×10⁸ cells, at least about 6×10⁸ cells, at least about 7×10⁸ cells, at least about 8×10⁸ cells, at least about 9×10⁸ cells, at least about 1×10⁹ cells, at least about 2×10⁹ cells, at least about 3×10⁹ cells, at least about 4×10⁹ cells, at least about 5×10⁹ cells, at least about 6×10⁹ cells, at least about 7×10⁹ cells, at least about 8×10⁹ cells, at least about 9×10⁹ cells, at least about 1×10¹⁰ cells, at least about 2×10¹⁰ cells, at least about 3×10¹⁰ cells, at least about 4×10¹⁰ cells, at least about 5×10¹⁰ cells, at least about 6×10¹⁰ cells, at least about 7×10¹⁰ cells, at least about 8×10¹⁰ cells, at least about 9×10¹⁰ cells, at least about 1×10¹¹ cells, at least about 2×10¹¹ cells, at least about 3×10¹¹ cells, at least about 4×10¹¹ cells, at least about 5×10¹¹ cells, at least about 6×10¹¹ cells, at least about 7×10¹¹ cells, at least about 8×10¹¹ cells, at least about 9×10¹¹ cells, or at least about 1×10¹² cells which are administered to a subject. In some embodiments, the population of immune cells can comprise at most about 5×10¹⁰ cells, at most about 4×10¹⁰ cells, at most about 3×10¹⁰ cells, at most about 2×10¹⁰ cells, at most about 1×10¹⁰ cells, at most about 9×10⁹ cells, at most about 8×10⁹ cells, at most about 7×10⁹ cells, at most about 6×10⁹ cells, at most about 5×10⁹ cells, at most about 4×10⁹ cells, at most about 3×10⁹ cells, at most about 2×10⁹ cells, at most about 1×10⁹ cells, at most about 9×10⁸ cells, at most about 8×10⁸ cells, at most about 7×10⁸ cells, at most about 6×10⁸ cells, at most about 5×10⁸ cells, at most about 4×10⁸ cells, at most about 3×10⁸ cells, at most about 2×10⁸ cells, at most about 1×10⁸ cells, at most about 9×10⁷ cells, at most about 8×10⁷ cells, at most about 7×10⁷ cells, at most about 6×10⁷ cells, at most about 5×10⁷ cells, at most about 4×10⁷ cells, at most about 3×10⁷ cells, at most about 2×10⁷ cells, at most about 1×10⁷ cells, at most about 9×10⁶ cells, at most about 8×10⁶ cells, at most about 7×10⁶ cells, at most about 6×10⁶ cells, at most about 5×10⁶ cells, at most about 4×10⁶ cells, at most about 3×10⁶ cells, at most about 2×10⁶ cells, at most about 1×10⁶ cells, at most about 9×10⁵ cells, at most about 8×10⁵ cells, at most about 7×10⁵ cells, at most about 6×10⁵ cells, at most about 5×10⁵ cells, at most about 4×10⁵ cells, at most about 3×10⁵ cells, at most about 2×10⁵ cells, or at most about 1×10⁵ cells. The population of immune cells can be administered to a subject in need thereof. For example, about 5×10¹⁰ cells may be administered to a subject. In some cases, a population of cells can be expanded to sufficient numbers for therapy. For example, 5×10⁷ cells can undergo rapid expansion to generate sufficient numbers for therapeutic use. Any number of cells can be administered to a subject, for example by infusion, for therapeutic use. A patient may be infused, for example, with a number of cells between about 1×10⁶ to 5×10¹², inclusive. A patient may be infused with as many cells that can be generated for them.

In any of the cells of the various aspects herein, the cell may exhibit specific binding to two antigens simultaneously present in a target cell. The antigen may be present on the target cell surface or, in some cases, can be an intracellular protein of a target cell that is displayed by another cell, such as in the context of MHC.

In various embodiments of the aspects herein, the antigen binding domain linked to the CAR may primarily mediate interaction between the immune cell and the target cell and the antigen binding domain linked to the modified TCR complex may primarily mediate an immune cell activity when the interaction between the immune cell and the target cell takes place. Immune cell activity, as previously described herein, can include clonal expansion of the immune cell; cytokine release by the immune cell; cytotoxicity of the immune cell; proliferation of the immune cell; differentiation, dedifferentiation or transdifferentiation of the immune cell; movement and/or trafficking of the immune cell; exhaustion and/or reactivation of the immune cell; and release of other intercellular molecules, metabolites, chemical compounds, or combinations thereof by the immune cell.

In an aspect, provided herein is a method of inducing activity of an immune cell and/or a target cell, comprising (a) expressing a system in an immune cell; and (b) contacting a target cell with the immune cell under conditions that induce activity of the immune cell and/or the target cell, wherein the system expressed in the immune cell comprises a chimeric antigen receptor (CAR) comprising a first antigen binding domain having binding specificity for a first epitope, a transmembrane domain, and an intracellular signaling domain devoid of a signaling domain of CD3 zeta; and a modified T cell receptor (TCR) complex comprising a second antigen binding domain linked to at least one of (i) at least one TCR chain selected from an alpha chain, a beta chain, a gamma chain and a delta chain of a T cell receptor; (ii) an epsilon chain, a delta chain, and/or a gamma chain of cluster of differentiation 3 (CD3); or (iii) a CD3 zeta chain.

Upon contacting the target cell with the immune cell expressing the system, the first antigen binding domain and/or the second antigen binding domain may bind to their respective epitopes. These epitopes, for example, are present on the target cell. The binding of the first antigen binding domain and/or the second antigen binding domain to their respective epitopes can activate cytotoxicity of the immune cell. In some cases, the cytotoxicity activated in the immune cell is enhanced when both the first antigen binding domain and the second antigen binding domain bind to their respective epitopes is enhanced as compared to a comparable immune cell lacking the system, a comparable immune cell lacking one or more components of the system (e.g., CAR, modified TCR complex), and/or a comparable immune cell expressing the system and wherein only one of the first antigen binding domain and the second antigen binding domain is bound to the respective epitope. The binding of the first antigen binding domain and/or binding of the second antigen binding domain to their respective epitopes can activate cytotoxicity of the immune cell and reduce a side effect associated with the cytotoxicity. In some cases, the reduction in the side effect associated with cytotoxicity is greater as compared to a comparable immune cell lacking the system, a comparable immune cell lacking one or more components of the system (e.g., CAR, modified TCR complex), and/or a comparable immune cell expressing the system and wherein only one of the first antigen binding domain and the second antigen binding domain is bound to the respective epitope. In some cases, the side effect which is reduced is cytokine release syndrome. The binding of the first antigen binding domain and/or binding of the second antigen binding domain to their respective epitopes can activate cytotoxicity of the immune cell and increase persistence of the cytotoxicity. In some cases, the persistence of cytotoxicity is increased as compared to a comparable immune cell lacking the system, a comparable immune cell lacking one or more components of the system (e.g., CAR, modified TCR complex), and/or a comparable immune cell expressing the system and wherein only one of the first antigen binding domain and the second antigen binding domain is bound to the respective epitope. In some cases, cytotoxicity of the immune cell induces death of a target cell.

In various embodiments of a method of inducing activity of the immune cell and/or target cell, the immune cell can be any of a variety of immune cells. In some cases, the immune cell comprises a lymphocyte. The lymphocyte can be T cell. Non-limiting examples of T cells include CD8+ T cells, CD4+ T cells, αβ T cells, γδ T cells, Vγ9δ2 T cells, Vδ1 T cells, Vδ3 T cells and Vδ5 T cells. In some cases, the lymphocyte expressing a subject system is a natural killer (NK) cell, effector T cells, memory T cells, cytotoxic T cells, and/or T helper cells. In some cases, the lymphocyte expressing a subject system is a KHYG cell such as KHYG-1 cell or a derivative thereof.

In various embodiments of a method of inducing activity of the immune cell and/or target cell, the target cell can be any of a variety of cell types. The target cell can be, for example, a cancer cell, a hematopoietic cell, or a solid tumor cell. The target cell can, in some cases, be a cell identified in one or more of heart, blood vessels, salivary gland, esophagus, stomach, liver, gallbladder, pancreas, intestine, colon, rectum, anus, endocrine gland, adrenal gland, kidney, ureter, bladder, lymph node, tonsils, adenoid, thymus, spleen, skin, muscle, brain, spinal cord, nerve, ovary, fallopian tube, uterus, vagina, mammary gland, testes, prostate, penis, pharynx, larynx, trachea, bronchi, lung, diaphragm, cartilage, ligaments, and tendon. The target cell can be a diseased cell.

In an aspect, the present disclosure provides a method of treating a cancer of a subject. The method comprises (a) administering to a subject an antigen-specific immune cell comprising a chimeric antigen receptor (CAR) comprising a first antigen binding domain and a modified T cell receptor (TCR) complex comprising a second antigen binding domain, wherein a target cell of a cancer of the subject expresses one or more antigens recognized by the first and/or second antigen binding domain, and wherein the immune cell binds specifically to the target cell, and (b) contacting the target cell with the antigen-specific immune cell via the first and/or second antigen binding domains under conditions that induces an immune cell activity of the immune cell against the target cell, thereby inducing death of the target cell of the cancer.

In an aspect, the present disclosure provides a method of treating a cancer of a subject, comprising (a) administering to a subject an antigen-specific immune cell, wherein the antigen-specific immune cell is a genetically modified immune cell expressing any system of the embodiments provided herein; and (b) contacting the target cell with the antigen-specific immune cell under conditions that induces an immune cell activity of the immune cell against a target cell of a cancer of the subject, thereby inducing death of the target cell of the cancer.

In some embodiments, a method of treating a cancer of a subject comprises genetically modifying an immune cell to yield the antigen-specific immune cell.

Upon contacting the target cell with the antigen-specific immune cell, immune cell activity against a target cell of a cancer of the subject can induce death of the target cell. An immune cell activity can be selected from the group consisting of: clonal expansion of the immune cell; cytokine release by the immune cell; cytotoxicity of the immune cell; proliferation of the immune cell; differentiation, dedifferentiation or transdifferentiation of the immune cell; movement and/or trafficking of the immune cell; exhaustion and/or reactivation of the immune cell; and release of other intercellular molecules, metabolites, chemical compounds, or combinations thereof by the immune cell. In some cases, the immune cell activity is cytotoxicity of the immune cell. Cytotoxicity of an immune cell against a target cell can yield at least about 5%, 10%, 15%, 20%, 25%, 30%, 35%, 40%, 45%, 50%, 55%, 60%, 65%, 70%, 75%, 80%, 85%, 90%, 95%, or up to about 100% reduction in a cancer of a subject. In some embodiments, an immune cell activity can be cytokine release by an immune cell. In some cases, cytokine is released by the immune cell. The amount of cytokine released by the immune cell can be at least 3%, 5%, 10%, 15%, 20%, 25%, 30%, 35%, 40%, 45%, 50%, 55%, 60%, 65%, 70%, 75%, 80%, 85%, 90%, 95%, or up to about 100% less than that of comparable immune cell lacking the system, a comparable immune cell lacking one or more components of the system (e.g., CAR, modified TCR), and/or a comparable immune cell in which only one of the first and second antigen binding domains are bound to their respective epitopes. In some cases, persistence of the immune cell activity is greater when both the first and second antigen binding domain bind their respective epitopes, as compared to binding of only the first antigen binding domain alone or binding of the second antigen binding domain alone.

In various embodiments of a method of treating a cancer of a subject, the immune cell can be any of a variety of immune cells. In some cases, the immune cell comprises a lymphocyte. The lymphocyte can be T cell. Non-limiting examples of T cells include CD8+ T cells and CD4+ T cells. In some cases, the lymphocyte is a natural killer (NK) cell. In some cases, the lymphocyte expressing a subject system is a KHYG cell such as KHYG-1 cell or a derivative thereof.

In various embodiments of a method of treating a cancer of a subject, the cancer can be any one of a variety of cancers. The cancer is, for example, bladder cancer, bone cancer, brain cancer, breast cancer, cervical cancer, colon cancer, esophageal cancer, gastric cancer, glioma, head and neck cancer, kidney cancer, leukemia, acute myeloid leukemia (AML), multiple myeloma, liver cancer, lung cancer, lymphoma, melanoma, mesothelioma, medulloblastoma, ovarian cancer, pancreatic cancer, prostate cancer, rectal cancer, skin cancer, testicular cancer, tracheal cancer, or vulvar cancer.

In an aspect, the present disclosure provides a composition comprising one or more polynucleotides that encodes (a) a chimeric antigen receptor (CAR) comprising a first antigen binding domain having binding specificity for a first epitope, a transmembrane domain, and an intracellular signaling domain; and (b) a modified T cell receptor (TCR) complex comprising a second antigen binding domain which exhibits specific binding to a second epitope, wherein said second antigen binding domain is linked to: at least one TCR chain selected from an alpha chain, a beta chain, a gamma chain and a delta chain of a T cell receptor; an epsilon chain, a delta chain, and/or a gamma chain of cluster of differentiation 3 (CD3); or a CD3 zeta chain. The composition can comprise one or more polynucleotides that encodes (a) a chimeric antigen receptor (CAR) comprising a first antigen binding domain having binding specificity for a first epitope, a transmembrane domain, and an intracellular signaling domain; and (b) a second antigen binding domain linked to: an alpha chain, a beta chain, a gamma chain, and/or a delta chain of a T cell receptor; an epsilon chain, a delta chain, and/or a gamma chain of cluster of differentiation 3 (CD3); or a CD3 zeta chain. In some embodiments, one or more polynucleotides comprises a promoter operably linked thereto. The one or more polynucleotides can comprise deoxyribonucleic acid (DNA) and/or ribonucleic acid (RNA). In some embodiments, one or more of the components of the system encoded by the one or more polynucleotides is joined by a linker that separates two or more nucleic acid coding regions. A linker can be a 2A sequence, a furin-V5-SGSGF2A, and the like.

In an aspect, the present disclosure provides a method of producing a modified immune cell, comprising genetically modifying the immune cell by expressing a composition provided herein in the immune cell, thereby producing said modified immune cell.

In various embodiments of the aspects herein, immune cells comprising a system provided herein can be used to induce death of a target cell. A variety of target cells can be killed using the systems and methods of the disclosure. A target cell to which this method can be applied includes a wide variety of cell types. A target cell can be in vitro. A target cell can be in vivo. A target cell can be ex vivo. A target cell can be an isolated cell. A target cell can be a cell inside of an organism. A target cell can be an organism. A target cell can be a cell in a cell culture. A target cell can be one of a collection of cells. A target cell can be a mammalian cell or derived from a mammalian cell. A target cell can be a rodent cell or derived from a rodent cell. A target cell can be a human cell or derived from a human cell. A target cell can be a prokaryotic cell or derived from a prokaryotic cell. A target cell can be a bacterial cell or can be derived from a bacterial cell. A target cell can be an archaeal cell or derived from an archaeal cell. A target cell can be a eukaryotic cell or derived from a eukaryotic cell. A target cell can be a pluripotent stem cell. A target cell can be a plant cell or derived from a plant cell. A target cell can be an animal cell or derived from an animal cell. A target cell can be an invertebrate cell or derived from an invertebrate cell. A target cell can be a vertebrate cell or derived from a vertebrate cell. A target cell can be a microbe cell or derived from a microbe cell. A target cell can be a fungi cell or derived from a fungi cell. A target cell can be from a specific organ or tissue.

A target cell can be a stem cell or progenitor cell. Target cells can include stem cells (e.g., adult stem cells, embryonic stem cells, induced pluripotent stem (iPS) cells) and progenitor cells (e.g., cardiac progenitor cells, neural progenitor cells, etc.). Target cells can include mammalian stem cells and progenitor cells, including rodent stem cells, rodent progenitor cells, human stem cells, human progenitor cells, etc. Clonal cells can comprise the progeny of a cell. A target cell can be in a living organism. A target cell can be a genetically modified cell.

A target cell can be a primary cell. For example, cultures of primary cells can be passaged 0 times, 1 time, 2 times, 4 times, 5 times, 10 times, 15 times or more. Cells can be unicellular organisms. Cells can be grown in culture.

A target cell can be a diseased cell. A diseased cell can have altered metabolic, gene expression, and/or morphologic features. A diseased cell can be a cancer cell, a diabetic cell, and/or an apoptotic cell. A diseased cell can be a cell from a diseased subject. Exemplary diseases can include blood disorders, cancers, metabolic disorders, eye disorders, organ disorders, musculoskeletal disorders, cardiac disease, and the like.

If the target cells are primary cells, they may be harvested, for example in in vitro experiments, from an individual by any method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g. normal saline, phosphate-buffered saline (PBS), Hank's balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

A target cell can be identified in one or more of heart, blood vessels, salivary gland, esophagus, stomach, liver, gallbladder, pancreas, intestine, colon, rectum, anus, endocrine gland, adrenal gland, kidney, ureter, bladder, lymph node, tonsils, adenoid, thymus, spleen, skin, muscle, brain, spinal cord, nerve, ovary, fallopian tube, uterus, vagina, mammary gland, testes, prostate, penis, pharynx, larynx, trachea, bronchi, lung, diaphragm, cartilage, ligaments, and tendon.

Non-limiting examples of cells which can be target cells include, but are not limited to, hematopoietic cells, lymphoid cells, such as B cell, T cell (Cytotoxic T cell, Natural Killer T cell, Regulatory T cell, T helper cell), Tumor infiltrating lymphocyte (TIL), Natural killer cell, cytokine induced killer (CIK) cells; myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

Of particular interest are cancer cells. In some embodiments, the target cell is a cancer cell. A cancer can be a solid tumor or a hematological tumor. A cancer can be metastatic. A cancer can be a relapsed cancer. Non-limiting examples of cancer cells include cells of cancers including Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof. In some embodiments, the targeted cancer cell represents a subpopulation within a cancer cell population, such as a cancer stem cell. In some embodiments, the cancer is of a hematopoietic lineage, such as a lymphoma. The first and/or second antigen binding domains can bind to epitopes present on antigens of cancer cells.

In some embodiments, the target cells can form a tumor. A tumor treated with the methods herein can result in stabilized tumor growth (e.g., one or more tumors do not increase more than 1%, 5%, 10%, 15%, or 20% in size, and/or do not metastasize). In some embodiments, a tumor is stabilized for at least about 1, 2, 3, 4, 5, 6, 7, 8, 9, 10, 11, 12, or more weeks. In some embodiments, a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. In some embodiments, the size of a tumor or the number of tumor cells is reduced by at least about 5%, 10%, 15%, 20%, 25, 30%, 35%, 40%, 45%, 50%, 55%, 60%, 65%, 70%, 75%, 80%, 85%, 90%, 95% or more as a result of treatment according to methods provided herein. In some embodiments, the tumor is completely eliminated, or reduced below a level of detection. In some embodiments, a subject remains tumor free (e.g. in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, a subject remains tumor free for at least about 1, 2, 3, 4, 5, 6, 7, 8, 9, 10, or more years after treatment.

Death of target cells can be determined by any suitable method, including, but not limited to, counting cells before and after treatment, or measuring the level of a marker associated with live or dead cells (e.g. live or dead target cells).

Degree of cell death can be determined by any suitable method. In some embodiments, degree of cell death is determined with respect to a starting condition. For example, an individual can have a known starting amount of target cells, such as a starting cell mass of known size or circulating target cells at a known concentration. In such cases, degree of cell death can be expressed as a ratio of surviving cells after treatment to the starting cell population. In some embodiments, degree of cell death can be determined by a suitable cell death assay. A variety of cell death assays are available and can utilize a variety of detection methodologies. Examples of detection methodologies include, without limitation, the use of cell staining, microscopy, flow cytometry, cell sorting, and combinations of these.

When a tumor is subject to surgical resection following completion of a therapeutic period, the efficacy of treatment in reducing tumor size can be determined by measuring the percentage of resected tissue that is necrotic (i.e., dead). In some embodiments, a treatment is therapeutically effective if the necrosis percentage of the resected tissue is greater than about 20% (e.g., at least about 30%, 40%, 50%, 60%, 70%, 80%, 90%, or 100%). In some embodiments, the necrosis percentage of the resected tissue is 100%, that is, no living tumor tissue is present or detectable.

In various embodiments of the aspects provided herein, exposing a target cell to or contacting a target cell with an immune cell or population of immune cells can be conducted either in vitro or in vivo. Exposing a target cell to an immune cell or population of immune cells generally refers to bringing the target cell in contact with the immune cell and/or in sufficient proximity such that an antigen (e.g., comprising an epitope) of a target cell (e.g., membrane bound or non-membrane bound) can bind to the antigen binding domain of the first antigen binding domain and/or the second antigen binding domain. Exposing a target cell to an immune cell or population of immune cells in vitro can be accomplished by co-culturing the target cells and the immune cells. Target cells and immune cells can be co-cultured, for example, as adherent cells or alternatively in suspension. Target cells and immune cells can be co-cultured in various suitable types of cell culture media, for example with supplements, growth factors, ions, etc. Exposing a target cell to an immune cell or population of immune cells in vivo can be accomplished, in some cases, by administering the immune cells to a subject, for example a human subject, and allowing the immune cells to localize to the target cell via the circulatory system. In some cases, an immune cell can be delivered to the immediate area where a target cell is localized, for example, by direct injection.

Exposing or contacting can be performed for any suitable length of time, for example at least 1 minute, at least 5 minutes, at least 10 minutes, at least 30 minutes, at least 1 hour, at least 2 hours, at least 3 hours, at least 4 hours, at least 5 hours, at least 6 hours, at least 7 hours, at least 8 hours, at least 12 hours, at least 16 hours, at least 20 hours, at least 24 hours, at least 2 days, at least 3 days, at least 4 days, at least 5 days, at least 6 days, at least 1 week, at least 2 weeks, at least 3 weeks, at least 1 month or longer.

In various embodiments of the aspects herein, a system provided herein is expressed in a host cell (e.g., an immune cell, e.g., an antigen-specific immune cell). The host cell can be a human cell. The host cell can be a non-human cell. A host cell can be autologous or allogeneic to a subject in need thereof. In some cases, a host cell can be xenogeneic. A host cell can be an immune cell such as a lymphocyte or myeloid cell. A host cell can be a T cell, B cell, NK cell, and the like. In some embodiments, the host cell can be a CD3+ cell, CD3− cell, a CD5+ cell, CD5− cell, a CD7+ cell, CD7− cell, a CD14+ cell, CD14− cell, CD8+ cell, a CD8− cell, a CD103+ cell, CD103− cell, CD11b+ cell, CD11b− cell, a BDCA1+ cell, a BDCA1− cell, an L-selectin+ cell, an L-selectin− cell, a CD25+, a CD25− cell, a CD27+, a CD27− cell, a CD28+ cell, CD28− cell, a CD44+ cell, a CD44− cell, a CD56+ cell, a CD56− cell, a CD57+ cell, a CD57− cell, a CD62L+ cell, a CD62L− cell, a CD69+ cell, a CD69− cell, a CD45RO+ cell, a CD45RO− cell, a CD127+ cell, a CD127− cell, a CD132+ cell, a CD132− cell, an IL-7+ cell, an IL-7− cell, an IL-15+ cell, an IL-15− cell, a lectin-like receptor G1 positive cell, a lectin-like receptor G1 negative cell, or an differentiated or de-differentiated cell thereof. In some embodiments, the host cell may be positive for two or more factors. For example, the host cell may be CD4+ and CD8+. In some embodiments, the host cell may be negative for two or more factors. For example, the host cell may be CD25−, CD44−, and CD69−. In some embodiments, the host cell may be positive for one or more factors, and negative for one or more factors. For example, the cell may be CD4+ and CD8−. In some embodiments, host cells may be selected for having or not having one or more given factors (e.g., cells may be separated based on the presence or absence of one or more markers described herein).

In some embodiments, host cells that are selected may also be expanded in vitro Selected and/or expanded host cells may be administered to a subject in need thereof. It should be understood that cells used in any of the methods disclosed herein may be a mixture (e.g., two or more different cells) of any of the cells disclosed herein. For example, a composition may comprise a mixture of different cells, for example T cells and B cells. The mixture can include, for example, a stem memory T_{SCM} cell comprising CD45RO (−), CCR7(+), CD45RA (+), CD62L+ (L-selectin), CD27+, CD28+ and IL-7Rα+, stem memory cells can also express CD95, IL-2Eβ, CXCR3, and LFA-1, and show numerous functional attributes distinctive of stem memory cells. The mixture can include, for example, central memory T_{CM} cells comprising L-selectin and CCR7, where the central memory cells can secrete, for example, IL-2, but not IFNγ or IL-4. The mixture can include, for example, effector memory TEM cells comprising L-selectin or CCR7 and produce, for example, effector cytokines such as IFNγ and IL-4.

A host cell can be obtained from a subject. In some cases, a host cell can be a population of T cells, NK cell, B cells, and the like obtained from a subject. T cells can be obtained from a number of sources, including PBMCs, bone marrow, lymph node tissue, cord blood, thymus tissue, and tissue from a site of infection, ascites, pleural effusion, spleen tissue, and tumors. In some embodiments, T cells can be obtained from a unit of blood collected from a subject using any number of techniques, such as Ficoll™ separation. In one embodiment, cells from the circulating blood of an individual are obtained by apheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media for subsequent processing steps.

In some embodiments, a population of immune cells provided herein can be heterogeneous. In some embodiments, cells used can be composed of a heterogeneous mixture of CD4 and CD8 T cells. Said CD4 and CD8 cells can have phenotypic characteristics of circulating effector T cells. Said CD4 and CD8 cells can also have a phenotypic characteristic of effector-memory cells. In some embodiment, cells can be central-memory cells.

In some embodiments, host cells include peripheral blood mononuclear cells (PBMC), peripheral blood lymphocytes (PBL), and other blood cell subsets such as, but not limited to, T cell, a natural killer cell, a monocyte, a natural killer T cell, a monocyte-precursor cell, a hematopoietic stem cell or a non-pluripotent stem cell. In some cases, the cell can be any immune cell, including any T-cell such as tumor infiltrating cells (TILs), such as CD3+ T-cells, CD4+ T-cells, CD8+ T-cells, or any other type of T-cell. The T cell can also include memory T cells, memory stem T cells, or effector T cells. The T cells can also be selected from a bulk population, for example, selecting T cells from whole blood. The T cells can also be expanded from a bulk population. The T cells can also be skewed towards particular populations and phenotypes. For example, the T cells can be skewed to phenotypically comprise, CD45RO (−), CCR7 (+), CD45RA (+), CD62L (+), CD27 (+), CD28 (+) and/or IL-7Ra (+). Suitable cells can be selected that comprise one of more markers selected from a list comprising: CD45RO (−), CCR7 (+), CD45RA (+), CD62L (+), CD27 (+), CD28 (+) and/or IL-7Ra (+). Host cells also include stem cells such as, by way of example, embryonic stem cells, induced pluripotent stem cells, hematopoietic stem cells, neuronal stem cells and mesenchymal stem cells. Host cells can comprise any number of primary cells, such as human cells, non-human cells, and/or mouse cells. Host cells can be progenitor cells. Host cells can be derived from the subject to be treated (e.g., patient). Host cells can be derived from a human donor. Host cells can be stem memory TSCM cells comprised of CD45RO (−), CCR7(+), CD45RA (+), CD62L+(L-selectin), CD27+, CD28+ and IL-7Rα+, said stem memory cells can also express CD95, IL-2Rβ, CXCR3, and LFA-1, and show numerous functional attributes distinctive of said stem memory cells. Host cells can be central memory TCM cells comprising L-selectin and CCR7, said central memory cells can secrete, for example, IL-2, but not IFNγ or IL-4. Host cells can also be effector memory TEM cells comprising L-selectin or CCR7 and produce, for example, effector cytokines such as IFNγ and IL-4.

A number of viral based systems have been developed for gene transfer into mammalian cells. For example, retroviruses, lentiviruses, and adenoviruses provide a convenient platform for gene delivery systems. A subject system can be inserted into a vector and packaged in retroviral particles using techniques known in the art. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells. They also have the added advantage of low immunogenicity.

In an aspect, a nucleic acid encoding a system comprising a modified TCR complex and/or CAR can be delivered virally or non-virally. In some embodiments, a nucleic acid encoding a system comprising a modified TCR complex and/or CAR can be delivered by a viral delivery system. Viral delivery systems (e.g., viruses comprising the pharmaceutical compositions of the disclosure) can be administered by direct injection, stereotaxic injection, intracerebroventricularly, by minipump infusion systems, by convection, catheters, intravenous, parenteral, intraperitoneal, and/or subcutaneous injection, to a cell, tissue, or organ of a subject in need. In some instances, cells can be transduced in vitro or ex vivo with viral delivery systems. The transduced cells can be administered to a subject having a disease. For example, a stem cell can be transduced with a viral delivery system comprising a pharmaceutical composition and the stem cell can be implanted in the patient to treat a disease. In some instances, the dose of transduced cells given to a subject can be about 1×10⁵ cells/kg, about 5×10⁵ cells/kg, about 1×10⁶ cells/kg, about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 5×10⁷ cells/kg, about 1×10⁸ cells/kg, or more in one single dose.

In some embodiments, a nucleic acid encoding a system comprising a modified TCR complex and/or CAR can be delivered by a non-viral delivery system. Non-viral delivery systems that can be used herein include but are not limited to DNA plasmids, RNA, naked nucleic acid, and nucleic acid complexed with a delivery vehicle such as a liposome or poloxamer. In some embodiments, the non-viral delivery system is a transposon-based delivery system. In some embodiments, the non-viral delivery system is a transposon-based delivery system selected from Sleeping Beauty (SB) transposon system, PiggyBac transposon system, and Tol2 transposon system.

A packaging cell line can be used to generate viral particles comprising a system provided herein. A packaging cell line can also be utilized to perform methods provided herein. Packaging cells that can be used include, but are not limited to, HEK 293 cells, HeLa cells, and Vero cells to name a few. In some cases, supernatant of the packaging cell line is treated by PEG precipitation for concentrating viral particles. In other cases, a centrifugation step can be used to concentrate viral particles. For example, a column can be used to concentration a virus during a centrifugation. In some cases, a precipitation occurs at no more than about 4° C. (for example about 3° C., about 2° C., about 1° C., or about 1° C.) for at least about 2 hours, at least about 3 hours, at least about 4 hours, at least about 6 hours, at least about 9 hours, at least about 12 hours, or at least about 24 hours. In some cases, viral particles can be isolated from the PEG-precipitated supernatant by low-speed centrifugation followed by CsCl gradient. The low-speed centrifugation can be about 4000 rpm, about 4500 rpm, about 5000 rpm, or about 6000 rpm for about 20 minutes, about 30 minutes, about 40 minutes, about 50 minutes or about 60 minutes. In some cases, viral particles are isolated from PEG-precipitated supernatant by centrifugation at about 5000 rpm for about 30 minutes followed by CsCl gradient.

A virus (e.g., lentivirus) can be introduced to a subject cell or to a population of subject cells at about, from about, at least about, or at most about 1-3 hrs., 3-6 hrs., 6-9 hrs., 9-12 hrs., 12-15 hrs., 15-18 hrs., 18-21 hrs., 21-23 hrs., 23-26 hrs., 26-29 hrs., 29-31 hrs., 31-33 hrs., 33-35 hrs., 35-37 hrs., 37-39 hrs., 39-41 hrs., 2 days, 3 days, 4 days, 5 days, 6 days, 7 days, 8 days, 9 days, 10 days, 14 days, 16 days, 20 days, or longer than 20 days after a stimulation or activation step, for instance anti-CD3, anti-CD28, or a combination thereof. In some cases, a viral vector encodes for a system, for example a CAR-T, modified TCR complex, or a combination thereof. In some cases, a viral vector encodes for a CAR-T. In some cases, a viral vector encodes for a modified TCR complex. An immune cell can be transduced with viral particles encoding for both a CAR and a modified TCR complex. An immune cell can be transduced with viral particles encoding for a CAR. An immune cell can be transduced with viral particles encoding for a modified TCR complex. A nucleic acid encoding a subject system can be inserted randomly into the genome of a cell. A nucleic acid encoding a system can encode its own promoter or can be inserted into a position where it is under the control of an endogenous promoter of a cell. Alternatively, a nucleic acid encoding a system can be inserted into a gene, such as an intron of a gene, an exon of a gene, a promoter, or a non-coding region. Expression of a system can be verified by an expression assay, for example, qPCR or by measuring levels of RNA in transduced cells. Expression level can be indicative also of copy number. For example, if expression levels are high, this can indicate that more than one copy of a nucleic acid encoding a system was integrated in a genome of a cell. Alternatively, high expression can indicate that a nucleic acid encoding a system was integrated in a highly transcribed area, for example, near a highly expressed promoter. Expression can also be verified by measuring protein levels, such as through Western blotting.

Cell viability of a subject cell or subject population of cells can be measured by fluorescence-activated cell sorting (FACS). In some cases, cell viability is measured after a viral or a non-viral vector comprising a nucleic acid encoding a subject system is introduced to a cell or to a population of cells. In some cases, at least about, or at most about, or about 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.8%, or 100% of the cells in a population of cells are viable after a viral vector is introduced to the cell or to the population of cells. In some cases, cell viability is measured at about, at least about, or at most about 4 hours, 6 hours, 8 hours, 10 hours, 12 hours, 18 hours, 20 hours, 24 hours, 30 hours, 36 hours, 40 hours, 48 hours, 54 hours, 60 hours, 72 hours, 84 hours, 96 hours, 108 hours, 120 hours, 132 hours, 144 hours, 156 hours, 168 hours, 180 hours, 192 hours, 204 hours, 216 hours, 228 hours, 240 hours, or longer than 240 hours after a viral vector is introduced to a cell and/or to a population of cells. In some cases, cell viability is measured at about, at least about, or at most about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 45 days, 50 days, 60 days, 70 days, 90 days, or longer than 90 days after a viral vector is introduced to a cell or population of cells. In some cases, cellular toxicity is measured at about, at least about, or at most about 4 hours, 6 hours, 8 hours, 12 hours, 18 hours, 24 hours, 30 hours, 36 hours, 42 hours, 48 hours, 54 hours, 60 hours, 66 hours, 72 hours, 78 hours, 84 hours, 90 hours, 96 hours, 102 hours, 108 hours, 114 hours, 120 hours, 126 hours, 132 hours, 138 hours, 144 hours, 150 hours, 156 hours, 168 hours, 180 hours, 192 hours, 204 hours, 216 hours, 228 hours, 240 hours, or longer than 240 hours after a viral vector is introduced to a cell or to a population of cells.

In some embodiments, one or more nucleic acids encoding a system comprising a modified TCR complex and/or CAR can be delivered by viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

In some embodiments, immune cells expressing a system provided herein are administered. Immune cells can be administered before, during, or after the occurrence of a disease or condition, and the timing of administering the immune cells can vary. For example, immune cells expressing a subject system can be used as a prophylactic and can be administered continuously to subjects with a propensity to conditions or diseases in order to prevent the occurrence of the disease or condition. The immune cells can be administered to a subject during or as soon as possible after the onset of the symptoms. The administration can be initiated within the first 48 hours of the onset of the symptoms, within the first 24 hours of the onset of the symptoms, within the first 6 hours of the onset of the symptoms, or within 3 hours of the onset of the symptoms. The initial administration can be via any suitable route, such as by any route described herein using any formulation described herein. Immune cells can be administered as soon as is practicable after the onset of a disease or condition is detected or suspected, and for a length of time necessary for the treatment of the disease, such as, for example, from about 1 month to about 3 months. The length of treatment can vary for each subject.

The compositions provided herein comprising immune cells expressing the subject system may be administered to a subject using known modes and techniques. Exemplary modes include, but are not limited to, intravenous injection. Other modes include, without limitation, intratumoral, intradermal, subcutaneous (S.C., s.q., sub-Q, Hypo), intramuscular (i.m.), intraperitoneal (i.p.), intra-arterial, intramedullary, intracardiac, intra-articular (joint), intrasynovial (joint fluid area), intracranial, intraspinal, and intrathecal (spinal fluids). Any known device useful for parenteral injection of infusion of the formulations can be used to affect such administration. Formulations comprising the subject compositions can be administered to a subject in an amount that is effective for treating and/or prophylaxis of the specific indication or disease. A physician can determine appropriate dosages to be used. Compositions comprising immune cells expressing a subject system may be independently administered 4, 3, 2, or once daily, every other day, every third day, every fourth day, every fifth day, every sixth day, once weekly, every eight days, every nine days, every ten days, bi-weekly, monthly and bi-monthly.

Compositions and methods provided herein can be combined with secondary therapies including cytotoxic/antineoplastic agents and anti-angiogenic agents. Cytotoxic/anti-neoplastic agents can be defined as agents who attack and kill cancer cells. Anti-angiogenic agents can also be used. Small molecules, including topoisomerases such as razoxane, a topoisomerase II inhibitor with anti-angiogenic activity, can also be used.

Immune cells comprising any system provided herein can be administered to a subject in conjunction with (e.g., before, simultaneously, or following) any number of relevant treatment modalities, including but not limited to treatment with agents such as antiviral therapy, cidofovir and interleukin-2, or Cytarabine (also known as ARA-C). In some cases, the subject immune cells can be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and irradiation. The engineered cell composition can also be administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In some cases, the subject immune cell compositions can be administered following B-cell ablative therapy such as agents that react with CD20, e.g., Rittman. For example, subjects can undergo standard treatment with high dose chemotherapy followed by peripheral blood stem cell transplantation. In certain embodiments, following the transplant, subjects can receive an infusion of immune cells, e.g., expanded immune cells comprising a subject system. Additionally, expanded immune cells can be administered before or following surgery.

Immune cells, compositions and methods provided herein can be used in combination with (e.g., before, simultaneously, or following) a T cell stimulating agent. T cell stimulating agents are molecules which can activate, expand, stimulate or modulate T cells. In some cases, the T cell stimulating agents are γδ T cell stimulating agents. In particular, said T cell stimulating agents include, but are not limited to isopentenyl pyrophosphate (IPP), 4-hydroxy-3-methyl-but-2-enyl pyrophosphate (HMB-PP), ethyl pyrophosphate (EPP), farnesyl pyrophosphate (FPP), dimethylallyl phosphate (DMAP), dimethylallyl pyrophosphate (DMAPP), ethyl-adenosine triphosphate (EPPPA), geranyl pyrophosphate (GPP), geranylgeranyl pyrophosphate (GGPP), isopentenyl-adenosine triphosphate (IPPPA), monoethyl phosphate (MEP), monoethyl pyrophosphate (MEPP), 3-formyl-1-butyl-pyrophosphate (TUBAg 1), X-pyrophosphate (TUBAg 2), 3-formyl-1-butyl-uridine triphosphate (TUBAg 3), 3-formyl-1-butyl-deoxythymidine triphosphate (TUBAg 4), monoethyl alkylamines, allyl pyrophosphate, crotoyl pyrophosphate, dimethylallyl-γ-uridine triphosphate, crotoyl-γ-uridine triphosphate, allyl-γ-uridine triphosphate, ethylamine, isobutylamine, sec-butylamine, iso-amylamine, nitrogen containing bisphosphonates, bisphosphonic acid (such as pamidronate, alendronate, zoledronate, risedronate, neridronate, ibandronate, incadronate, olpadronate, solvadronate, minodronate, EB1053, etidronate, clodronate, tiludronate and medronate), bromohydrin pyrophosphate (BrHPP), Concanavalin A (ConA), or any analog thereof.

In some cases, for example, in the compositions, formulations and methods of treating cancer, the unit dosage of the composition or formulation administered can be 5, 10, 15, 20, 25, 30, 35, 40, 45, 50, 55, 60, 65, 70, 75, 80, 85, 90, 95 or 100 mg. In some cases, the total amount of the composition or formulation administered can be 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 40, 50, 60, 70, 80, 90, or 100 g.

Provided herein can also be pharmaceutical compositions comprising subject cells and subject systems. In an aspect, a pharmaceutical composition comprises a population of cells comprising a subject system in unit dosage form. In an aspect, a pharmaceutical composition comprises a population of cells that comprise a gamma delta T cell that comprises a subject system. In an aspect, a pharmaceutical composition comprises a population of cells that comprise an alpha beta T cell that comprises a subject system.

Pharmaceutical compositions comprising systems or immune cells comprising systems described herein can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the condition. Amounts effective for this use can vary based on the severity and course of the disease or condition, previous therapy, the subject's health status, weight, and response to the drugs, and the judgment of the treating physician.

Multiple therapeutic agents can be administered in any order or simultaneously. If simultaneously, the multiple therapeutic agents can be provided in a single, unified form, or in multiple forms, for example, as multiple separate pills. The molecules can be packed together or separately, in a single package or in a plurality of packages. One or all of the therapeutic agents can be given in multiple doses. If not simultaneous, the timing between the multiple doses may vary to as much as about a month.

Systems described herein can be administered before, during, or after the occurrence of a disease or condition, and the timing of administering the composition can vary. For example, the pharmaceutical compositions can be used as a prophylactic and can be administered continuously to subjects with a propensity to conditions or diseases in order to prevent the occurrence of the disease or condition. The pharmaceutical compositions can be administered to a subject during or as soon as possible after the onset of the symptoms. The administration of the molecules can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A system can be administered as soon as is practicable after the onset of a disease or condition is detected or suspected, and for a length of time necessary for the treatment of the disease, such as, for example, from about 1 month to about 3 months. The length of treatment can vary for each subject.

### EXAMPLES

Various aspects of the disclosure are further illustrated by the following non-limiting examples.

### Example 1: Design of the TCR+Co-Stimulatory Only CAR Constructs

This example describes the design of exemplary TCR+co-stimulatory only CAR constructs. 12 constructs were designed, each comprising one polynucleotide as follows:

Construct 1 (Anti-CLL-1 TCRε+Anti-CD33 Costimulatory CAR): The polynucleotide comprises from the N-terminus to the C terminus is CD3ε leading peptide-anti-CLL-1 sdAb-(G4S)₃-CD3ε-T2A-CD8 leading peptide-anti-CD33 sdAb-CD8 hinge-CD8 transmembrane-CD27 costimulatory domain;

Construct 2 (Anti-CLL-1 TCRδ+Anti-CD33 Costimulatory CAR): The polynucleotide comprises from the N-terminus to the C terminus is CD3δ leading peptide-anti-CLL-1 sdAb-(G4S)₃-CD3δ-T2A-CD8 leading peptide-anti-CD33 sdAb-CD8 hinge-CD8 transmembrane-CD27 costimulatory domain;

Construct 3 (Anti-CLL-1 TCRγ+Anti-CD33 Costimulatory CAR): The polynucleotide comprises from the N-terminus to the C terminus is CD3γ leading peptide-anti-CLL-1 sdAb-(G4S)₃-CD3γ-T2A-CD8 leading peptide-anti-CD33 sdAb-CD8 hinge-CD8 transmembrane-CD27 costimulatory domain;

Construct 4 (Anti-CLL-1 Tandem TCRεε+Anti-CD33 Costimulatory CAR): The polynucleotide comprises from the N-terminus to the C terminus is CD3ε leading peptide-anti-CLL-1 sdAb-(G4S)₃-anti-CLL-1 sdAb-(G4S)₃-CD3ε-T2A-CD8 leading peptide-anti-CD33 sdAb-CD8 hinge-CD8 transmembrane-CD27 costimulatory domain;

Construct 5 (Anti-CLL-1 Tandem TCRδδ+Anti-CD33 Costimulatory CAR): The polynucleotide comprises from the N-terminus to the C terminus is CD3δ leading peptide-anti-CLL-1 sdAb-(G4S)₃-anti-CLL-1 sdAb-(G4S)₃-CD3δ-T2A-CD8 leading peptide-anti-CD33 sdAb-CD8 hinge-CD8 transmembrane-CD27 costimulatory domain;

Construct 6 (Anti-CLL-1 Tandem TCRγγ+Anti-CD33 Costimulatory CAR): The polynucleotide comprises from the N-terminus to the C terminus is CD3γ leading peptide-anti-CLL-1 sdAb-(G4S)₃-anti-CLL-1 sdAb-(G4S)₃-CD3γ-T2A-CD8 leading peptide-anti-CD33 sdAb-CD8 hinge-CD8 transmembrane-CD27 costimulatory domain;

Construct 7 (Anti-CLL-1 TCRε+Anti-CD33 Costimulatory Tandem CAR): The polynucleotide comprises from the N-terminus to the C terminus is CD3ε leading peptide-anti-CLL-1 sdAb-(G4S)₃-CD3ε-T2A-CD8 leading peptide-anti-CD33 sdAb-(G4S)₃-anti-CD33 sdAb-CD8 hinge-CD8 transmembrane-CD27 costimulatory domain;

Construct 8 (Anti-CLL-1 TCRδ+Anti-CD33 Costimulatory Tandem CAR): The polynucleotide comprises from the N-terminus to the C terminus is CD3δ leading peptide-anti-CLL-1 sdAb-(G4S)₃-CD3δ-T2A-CD8 leading peptide-anti-CD33 sdAb-(G4S)₃-anti-CD33 sdAb-CD8 hinge-CD8 transmembrane-CD27 costimulatory domain;

Construct 9 (Anti-CLL-1 TCRγ+Anti-CD33 Costimulatory Tandem CAR): The polynucleotide comprises from the N-terminus to the C terminus is CD3γ leading peptide-anti-CLL-1 sdAb-(G4S)₃-CD3γ-T2A-CD8 leading peptide-anti-CD33 sdAb-(G4S)₃-anti-CD33 sdAb-CD8 hinge-CD8 transmembrane-CD27 costimulatory domain;

Construct 10 (Anti-CLL-1 Parallel TCRεδ+Anti-CD33 Costimulatory CAR): The polynucleotide comprises from the N-terminus to the C terminus is CD3ε leading peptide-anti-CLL-1 sdAb-(G4S)₃-CD3ε-T2A-CD3δ leading peptide-anti-CLL-1 sdAb-(G4S)₃-CD3δ-T2A-CD8 leading peptide-anti-CD33 sdAb-CD8 hinge-CD8 transmembrane-CD27 costimulatory domain;

Construct 11 (Anti-CLL-1 Parallel TCRεγ+Anti-CD33 Costimulatory CAR): The polynucleotide comprises from the N-terminus to the C terminus is CD3ε leading peptide-anti-CLL-1 sdAb-(G4S)₃-CD3ε-T2A-CD3γ leading peptide-anti-CLL-1 sdAb-(G4S)₃-CD3γ-T2A-CD8 leading peptide-anti-CD33 sdAb-CD8 hinge-CD8 transmembrane-CD27 costimulatory domain;

Construct 12 (Anti-CLL-1 Parallel TCRδγ+Anti-CD33 Costimulatory CAR): The polynucleotide comprises from the N-terminus to the C terminus is CD3γ leading peptide-anti-CLL-1 sdAb-(G4S)₃-CD3γ-T2A-CD3δ leading peptide-anti-CLL-1 sdAb-(G4S)₃-CD3δ-T2A-CD8 leading peptide-anti-CD33 sdAb-CD8 hinge-CD8 transmembrane-CD27 costimulatory domain;

The anti-CLL-1 sdAb sequence used for the above system corresponds to SEQ ID NO. 41 listed in Table 5. The anti-CD33 sdAb sequence used for the above system corresponds to SEQ ID NO.23 listed in Table 2.

The polynucleotide was gene synthesized and inserted into lentivirus vector for modified TCR and/or CAR transduction. Non-limiting exemplary sequences of leading peptide, hinge and transmembrane domain below can comprise from about 60%, 70%, 80%, 90%, 95%, 97%, 99%, or 100% identity to the sequences below:

(CD8 leader)
SEQ ID NO: 1
MALPVTALLLPLALLLHAARP
(CD3ϵ leader)
SEQ ID NO: 2
MQSGTHWRVLGLCLLSVGVWGQ
(CD3γ leader)
SEQ ID NO: 3
MEQGKGLAVLILAIILLQGTLA
(CD3δ leader)
SEQ ID NO: 4
MEHSTFLSGLVLATLLSQVSP
(CD8 hinge)
SEQ ID NO: 5
TTTPAPRPPTPAPTIASQPLSLRPEACRPAAGGAVHTRGLDFACD
(CD8 transmembrane)
SEQ ID NO: 6
IYIWAPLAGTCGVLLLSLVITLYC

### Example 2: Viral Transfection and Viral Particle Generation

To generate viral particles comprising polynucleic acids encoding any of the systems disclosed herein lentivirus packaging plasmid mixture including pMDLg/pRRE (Addgene #12251), pRSV-Rev (Addgene #12253), and pMD2.G (Addgene #12259) was pre-mixed with a PLVX-EF1A (including target system) vector at a pre-optimized ratio with polyetherimide (PEI), mixed properly, and incubated at room temperature for 5 minutes. The transfection mix was added dropwise to 293-T cells and mixed gently. Transfected 293-T cells were incubated overnight at 37° C. and 5% CO₂. 24 hours post transfection, supernatants were collected and centrifuged at 4° C., 500 g for 10 min to remove any cellular debris. Centrifuged supernatants were filtered through a 0.45 μm PES filter to concentrate the viral supernatants post ultracentrifugation. After centrifugation, the supernatants were carefully discarded and the virus pellets were rinsed with pre-chilled DPBS. The concentration of virus was measured. Virus was aliquoted and stored at −80° C. Viral titer was determined by functional transduction on a T cell line.

Briefly, the lentiviral vector was modified using pLVX-Puro (Clontech #632164) by replacing the original promoter with human elongation factor 1α promoter (hEF1α) and by removing the puromycin resistance gene with EcoRI and BamHI by GenScript. PLVX-EF1A, was further subjected to the lentivirus packaging procedure as described above.

### Example 3: Immune Cell Preparation

Alpha/beta Leukocytes were collected in R10 medium, then mixed with 0.9% NaCl solution at a 1:1 (v/v) ratio. 3 mL lymphoprep medium was added to a 15 mL centrifuge tube. Lymphoprep was slowly layered forming 6 mL of diluted lymphocyte mix. The lymphocyte mix was centrifuged at 800 g for 30 minutes without brakes at 20° C. Lymphocyte buffy coat was then collected with a 200 μL pipette. The harvested fraction was diluted at least 6 fold of 0.9% NaCl or R10 to reduce the density of the solution. The harvested fraction was then centrifuged at 250 g for 10 minutes at 20° C. The supernatant was aspirated completely, and 10 mL of R10 was added to the cell pellet. The mixture was further centrifuged at 250 g for 10 minutes at 20° C. The supernatant was then aspirated. 2 mL 37° C. pre-warmed R10 with 100 IU/mL IL-2 was added to the cell pellet, and the cell pellet was re-suspended softly. Cells were quantified and the PBMC sample was ready for experimentation. Human T cells were purified from PBMCs using Miltenyi Pan T cell isolation kit (Cat #130-096-535).

The prepared alpha/beta T cells were subsequently pre-activated for 48 hours with human T cell Activation/Expansion kit (Milteny #130-091-441) by using one loaded anti-Biotin MACSiBead Particle per two cells (bead-to-cell ratio 1:2).

Gamma/delta T cells were prepared by addition of 5 μM Zoledronate and 1000 IU/mL IL-2 to PBMCs and cultured for 14 days with periodical change of media supplemented with 1000 IU/mL IL-2. Alternatively, gamma/delta T cells were isolated from PBMC or umbilical cord blood (UCB) and then stimulated by anti-gamma/delta TCR antibody and anti-CD3 (OKT3) followed by co-incubation of K562-based artificial antigen-presenting cells (aAPCs) at an 1:2 ratio for at least 10 days.

### Example 4: T Cell Modification

### (1) Alpha/Beta T Cell Transduction

The pre-activated alpha/beta T cells were collected and re-suspended in 1640 medium containing 300 IU/mL IL-2. A lentiviral vector encoding the system of Example 1 was diluted to MOI=5 with the same medium and infected with 1E+06 activated T cells. The pre-activated T cells were transduced with lentivirus stock in the presence of 8 μg/ml polybrene with centrifugation at 1000 g, 32° C. for 1 h. The transduced cells were then transferred to the cell culture incubator for transgene expression under suitable conditions. The next day, the transduced cells were centrifuged and replaced with fresh media, the cells concentration was measured every 2 days, and fresh media were added to continue the expansion.

### (2) Gamma/Delta T Cell Transduction

PBMCs were isolated by density centrifugation (lymphoprep) from leukapheresis material and cryopreserved. PBMCs were resuscitated and activated with zoledronic acid (5 μM) in cell culture media AIM-V supplemented with IL-2 (1000 IU/ml) and 5% human AB serum and kept in a humidified chamber (37° C., 5% CO₂). Forty-eight hours post-activation, cells were transduced with lentiviral vectors encoding the system of Example 1 at an MOI of 5 with 5 pg/ml polybrene. Such transduction procedure was repeated the next day followed by replenishment of fresh media containing IL-2 (1000 IU/ml) the day after the second transduction. Cells are cultured in AIM-V supplemented with IL-2 (1000 IU/ml) in a humidified chamber with periodical change of media as determined by the pH of the culture media for further expansion. Cells are harvested 10 days post-transduction and the total number, purity and transduction efficiency was determined. Cells were further enriched with a negative TCRγ/δ+ T cell isolation kit (Miltenyi Biotec) before future applications or cryopreserved.

### Example 5: Quantification of Receptor Expression

On day 3 and onwards (typically day 3, 7 and 14) post transduction, cells are evaluated for expression of the system of Example 1 by flow cytometry. An aliquot of cells is collected from the culture, washed, pelleted, and resuspended in diluted Ab (eBioscience Anti-Mouse TCR beta PE and anti-CAR Ab) 1/100 in PBS+0.5% FBS 50-100 ul per sample. Resuspended cells are in about 50 to 100 ul of Ab. Cell are incubated at 4° C. for 30 minutes. Viability dye eFluor780 or SYTOX Blue viability stain is also added according to manufacturer's instructions. Post incubation, cells are washed twice in PBS and resuspended in 100 to 200 ul PBS for analysis. The mean fluorescence of the system is quantified by flow cytometry.

For anti-CLL-1 staining, cells were stained with PE-labeled mouse-anti-human CLL-1 antibodies (BioLegend, clone number 50C1). For anti-CD33 staining, cells were stained with APC-labeled mouse-anti-human CD33 antibodies (BioLegend, clone number WM53). Flow cytometry analysis for all experiments was performed by using FlowJo (Tree Star, Inc.).

### Example 6: Cytotoxicity Assay

Cytotoxicity of 12 designed TCR+co-stimulatory CARs as well as their control T cells were determined in a 20 h co-culture assay. In the experiments, the effector cells were centrifugally collected, then diluted to the desired concentrations with 1640 phenol red free medium (Invitrogen) with 2% heat inactivated FBS (Invitrogen). The target cells, U937, exhibited strong expression of two target antigens CLL-1 and CD33. The effector cells were co-cultured at an effector to target ratios of 1:5 (E:T=5:1) at 37° C. for 48 h in 24 well plate. Additional wells contained assay buffer only (1640 phenol red free medium plus 2% hiFBS), target cell only (T), effector cell only (E) and max release of target cell (1% solution of triton-X 100). Each condition was performed in triplicate, and the cytotoxicity of effector cells was detected by LDH assay kit (Roche). After completion of the 20 hr co-culture, the assay plate was centrifuged, and supernatant collected in a new 96-w plate. The supernatant plate was diluted with an equal volume of the LDH assay reagent according to the manufacture's manual. The assay plate was incubated for about 30 min at 15° C.˜25° C. The absorbance of the plate was measured at 492 nm and 650 nm using Flexstation reader (Molecular Devices) and calculated as previously described. These effector cells went through two rounds of target cell stimulation to determine their anti-tumor toxicity levels in the context of repeated antigen stimulation.

Results show that the TCR+co-stimulatory CAR (without CD3zeta signal) only design displayed approximately the same level of anti-tumor cytotoxicity as compared with TCR only, CAR (with CD3zeta signal) only, and tested tandem designs, FIG. 5. However, the anti-tumor toxicity began to differentiate for different designs at the second round of antigen stimulation. First, the designs in which the anti-CLL-1 domain is fused to ε or γ subunits of CD3 within TCR complex showed better anti-tumor cytotoxicity as compared to δ counterparts. Nevertheless, TCR+costimulatory only CAR (without CD3zeta signal) displayed better in vitro efficacy during the second-round the antigen stimulation as compared to TCR only and co-stimulatory CAR (without CD3zeta signal) counterparts regardless of which CD3 subunits of TCR complex was utilized of anti-CLL-1 domain fusion (FIG. 5A, Figure B and Figure C). Second, when two anti-CLL-1 domains are fused to CD3 subunits within the TCR complex, significant improvement in anti-tumor cytotoxicity for TCR only designs for tandem designs was observed. In addition, when anti-CLL-1 binding domains are fused to the CD3 subunit with a TCR complex in parallel, stronger anti-tumor cytotoxicity was observed (FIG. 5D). Interestingly, the deficiencies found with the CD3δ subunits fusion with anti-CLL-1 domains can be rescued with either tandem or parallel designs. Based on the above findings in γδT cells, similar assays were performed using αβT cells with a focus on designs concerning CD3ε subunits. Results show that αβT cells comprising subject TCR+costimulatory only CAR (without CD3zeta signal) displayed better in vitro efficacy during the second-round of the antigen stimulation as compared to TCR only and co-stimulatory CAR (without CD3zeta signa). In addition, an improvement of anti-tumor cytotoxicity was found with tandem and parallel designs, similar to that of γδT cells (FIG. 5E).

### Example 7: Cytokine Release Assay

Gamma/delta or alpha/beta T cells with different designs specified in Example 1 were incubated with U937 cells at a ratio of 1:5 for 48 hours. The supernatants of the co-culture were collected for cytokine release analysis using commercial kits including Human IFN gamma kit (Cisbio, Cat #62HIFNGPEH), Human TNF alpha kit (Cisbio, Cat #62HTNFAPEH) and Human GM-CSF kit, Cisbio, Cat #62HGMCSFPEH. Cellular supernatants and an ELISA standard were dispensed directly into the assay plate for cytokine detection utilizing HTRF® reagents. The antibodies labeled with the HTRF donor and acceptor are pre-mixed and added in a single dispensing step. The ELISA standard curve is generated using a Four-Parameter Logistic (4PL) curve. Regression using the standard curve enables accurate measurement of an unknown sample concentration across a wider range of concentrations than linear analysis, which is suitable for analysis of biological systems such as cytokine release.

Cytokine production with αβT cells was much stronger than γδT cells with the same molecular designs, FIG. 6. This indicates that γδT cells comprising designs outlined in Example 1 can be safely administered to subjects. TCR+costimulatory only CAR (without CD3zeta signal) in γδT cells produced significantly less GM-TNF-α than TCR+ CAR (with CD3zeta signal), further demonstrating the safety features of TCR+costimulatory only CAR design in γδT cells (FIG. 6B). Additionally, both designs displayed similar level of IFN-γ, ensuring their anti-tumor cytotoxicity and functionality.

### Example 8: Colony Forming Unit (CFU) Assay

To evaluate potential toxicities, if any, of subject molecular designs outlined in Example 1 cytotoxicity of select constructs comprising anti-CD33 domains were used in a CFU assay against normal hematopoietic cells derived from CD34-enriched normal cord blood (CB) samples. CD34+ cord blood (CB) cells (HemaCare, Catalog: CB34C-2) were positively enriched and co-cultured with CD33 CAR-T cells or media alone (untreated) for 6 hours at an E:T (CAR-T: CB cells) of 10:1. The mixed cells were then plated in METHOCULT™ H4034 Optimum medium with a total cell number of 5000 (n=3), cultured for 5-7 days and scored for the presence of total colony forming unit 9 (CFU). CD123-specific CAR-T cells were used as positive controls. Anti-BCMA CAR-T and untransduced T cells were used as negative controls. Data represent mean values ±SEM of colony in triplicated petri dishes for each sample.

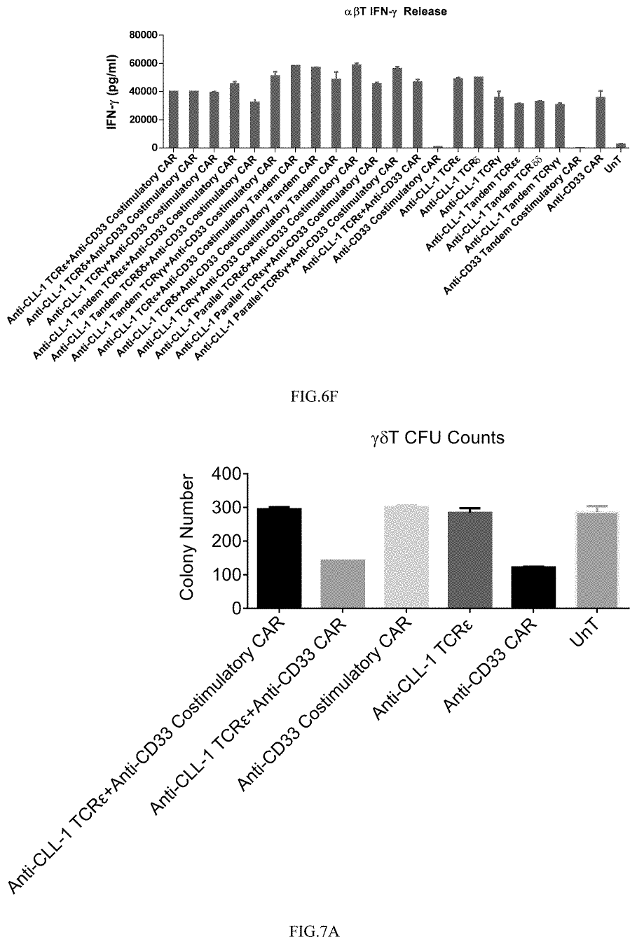

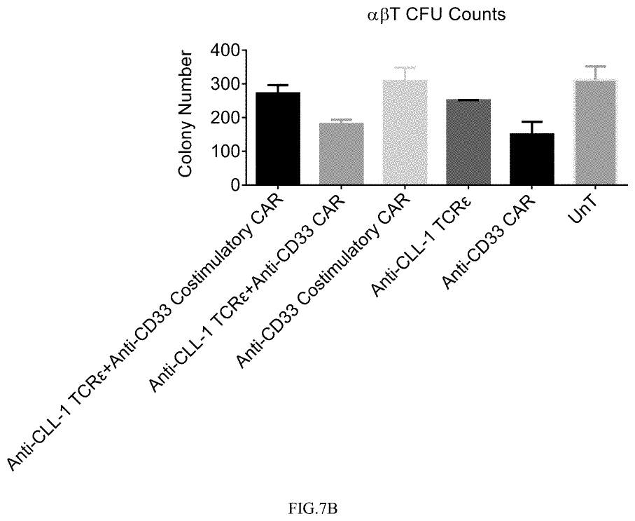

Results show that regardless of γδT cells or αβT cells, TCR+costimulatory only anti-CD33 CAR (without CD3zeta signal) showed the highest number of colonies counted, similar to that of costimulatory only anti-CD33 CAR (without CD3zeta signal) and unmodified T cells, FIG. 7. On the other hand, significantly less numbers of colonies were found with TCR+anti-CD33 CAR (with CD3zeta signal) and anti-CD33 CAR (with CD3zeta signal). These findings suggest that TCR+costimulatory only CAR (without CD3zeta signal) cells can be safely pursued as a therapy for cancer, such as AML, without the potential toxicity associated with HSC targeting.

A wide variety of antigen binding domain sequences are applicable for constructing the vectors constructs and systems disclosed herein, see e.g., WO2017/025038, which is incorporated herein in its entirety. Non-limiting exemplary sequences can comprise from about 60%, 70%, 80%, 90%, 95%, 97%, 99%, or 100% identity to sequences shown in Table 2 to Table 5 as follows:

TABLE 2
The sequences of the anti-BCMA V_{H}H

| SEQ ID NOs | Sequence |
|---|---|
| 7 | QVQLVESGGGLVQPGGSLRLSCEASGFTLDYYAIGWFRQAPGKEREGV |
| | ICISRSDGSTYYADSVKGRFTISRDNAKKTVYLQMISLKPEDTAAYYCA |
| | AGADCSGYLRDYEFRGQGTQVTVSS |
| 8 | QVKLEESGGRLVQPRGSLRLSCAGSGRTFSTYGMAWFRQAPGKEREFV |
| | ASKASMNYSGRTYYADSVKGRFTIARDNAKNMVFLQMNNLKPEDTA |
| | VYYCAAGTGCSTYGCFDAQIIDYWGKGTLVTVSS |
| 9 | EVQLVESGGGLVQAGGSLRLSCAASGRTFTMGWFRQAPGKEREFVAAI |
| | SLSPTLAYYAESVKGRFTISRDNAKNTVVLQMNSLKPEDTALYYCAAD |
| | RKSVMSIRPDYWGQGTQVTVSS |
| 10 | AVQLVDSGGGLVQPGGSLRLSCVASGGIFVINAMGWYRQAPGKQREL |
| | VASIRGLGRTNYDDSVKGRFTISRDNANNTVYLQMNSLEPEDTAVYYC |
| | TVYVTLLGGVNRDYWGQGTQVTVSS |
| 11 | EVQLVESGGGLVQAGGSLRLSCAASGRTFSSIVMGWFRQAPGKEREFV |
| | GAIMWNDGITYLQDSVKGRFTIFRDNAKNTVYLQMNSLKLEDTAVYY |
| | CAASKGRYSEYEYWGQGTQVTVSS |
| 12 | EVQLVESGGGVVQAGGSLTVSCTASGFTFDRAVIVWFRQAPGKGREG |
| | VSFIKPSDGTIYYIDSLKGRFTISSDIAKNTVYLQMKSLESEDSAVYYCA |
| | ASPEDWYTDWIDWSIYRWQHWGQGTQVTVSS |
| 13 | EVQLVESGGGMVQAGDSLRLSCVQSTYTVNSDVMGWFRQAPGKEREF |
| | VGAIMWNDGITYLQDSVKGRFTIFRDNAKNTVYLQMNSLKLEDTAVY |
| | YCAASKGRYSEYEYWGQGTQVTVSS |
| 14 | AVQLVESGGGLVQAGDSLRLSCTASGATLTNDHMAWFRQAPGKGREF |
| | VAAIDWSGRTTNYADPVEGRFTISRNNAKNTVYLEMNSLKLEDTAVY |
| | YCAVLRAWISYDNDYWGQGTQVTVSS |
| 15 | QVQLVESGGGLVQAGGSLRLSCAASGGTLSKNTVAWFRQAPGKERGF |
| | VASITWDGRTTYYADSVKGRFTISRDNAKNTVYLQMNSLKPEDTAVY |
| | VCADLGKWPAGPADYWGQGTQVTVSS |
| 16 | QVKLEESGGGLVQAGRSLRLSCAASEHTFSSHVMGWFRQAPGKERESV |
| | AVIGWRDISTSYADSVKGRFTISRDNAKKTLYLQMNSLKPEDTAVYYC |
| | AARRIDAADFDSWGQGTQVTVSS |
| 17 | AVQLVESGGGLVQAGDSLRLTCTASGRAFSTYFMAWFRQAPGKEREF |
| | VAGIAWSGGSTAYADSVKGRFTISRDNAKNTVYLQMNSLKSEDTAVY |
| | YCASRGIEVEEFGAWGQGTQVTVSS |

TABLE 3
The sequences of the anti-CD19 V_{H}H

| SEQ ID NO | Sequence |
|---|---|
| 18 | QVKLEESGGELVQPGGPLRLSCAASGNIFSINRMGWYRQAPGKQRAFV |
| | ASITVRGITNYADSVKGRFTISVDKSKNTIYLQMNALKPEDTAVYYCNA |
| | VSSNRDPDYWGQGTQVTVSS |
| 19 | QVKLEESGGGLVQAGESLRLSCAASGHTLSAYTMGWFRQAPEREREFV |
| | AAITRSGGRTSY |
| | GDSVKGRFTISRDTAKNTVYLQMNSLKPEDTAVYYCAADLRYRTVVN |
| | GLADYWGQGTQVTVSS |
| 20 | QVKLEESGGGLVQAGGSLRLSCAASGRSFSNYDMGWFRQAPGKEREF |
| | VARISRRGDSTYYADSVKGRFIISRDNAKNTVYLQMNSLKPEDTAVYY |
| | CAARWRGSREIDYWGQGTQVTVSS |

TABLE 4
The sequences of the anti-CD33 V_{H}H

| SEQ ID NO | Sequence (CDRs are underlined) |
|---|---|
| 21 | QVQLAESGGGSVQAGGSLRLSCAASGYTYSSKRMGWFRQAPGKKREG |
| | VAGIVTEDGTTYYADSVKGRFTISQDNAKNTVYLQMNSLKPEDTATYY |
| | CAAGTTYGGLNDWWYTLHAGGYNYWGQGTQVTVSS |
| 22 | EVQLAESGGGLVQAGGSLRLSCTASGFTFDDYVMGWFRQAPGKEREG |
| | VSCISWSGDTTYYADSVKGRFTASRDNAKNTLYLQMNSLKPEDTAMY |
| | YCAADQGKCSLASAEPDDMDYWGRGTLVTVSS |
| 23 | EVQLAESGGGSVQAGGSLRLSCAASGVTFSSTSVAWFRQAPGKEREGV |
| | AYIYTGDSSTYYADSVKGRFTIAQDNAKNAVYLQMNSLKPEDTAMYY |
| | CAADGFLLNHRSYQYWGQGTQVTVSS |
| 24 | EVQLVESGGGSVQAGGSLRLSCAASGYTYSINCMGWFRQAPGKEREG |
| | VAVISTGGGRTDYRDSVKGRFTISQDNAKNTVYLQMNSLKPEDTAMY |
| | YCAGKTTYPGYGCGLGRSAYNYWGQGTQVTVSS |
| 25 | QIQLVESGGGSVQAGGSLRLSCVASGYIGGHYYMGWFRQAPGKEREG |
| | VAAIDIDSDGRTRYAGSVQGRFTISQDNAKNTLHLQMSSLKPEDTGMY |
| | YCAVGVGWVPARLTPQAVSYWGKGTLVTVSS |
| 26 | EVQLVESGGGSVQAGGSLRLSCVASGYTYGINCMGWFRQAPGKEREGI |
| | AAISTGGGTTGVADSVKGRFTISQDNAKNTVYLQMNSLKPEDTAMYY |
| | CAARSTYSGYACVYSEVNGYNYRGQGTQVTVSS |
| 27 | EVQLVESGGGSVQAGGSLRLSCVASGYTWCRYDMSWYRQAPGKEREF |
| | VSGFDNDGTTSYADSVKGRFTISQDNDKNTVYLQMNSLKPEDTAMYY |
| | CKEDRSRGSLDGRVCPLSYDNWGQGTQVTVSS |
| 28 | EVQLVESGGGLVQAGGSLRLSCTASGFTFDDYVMGWFRQAPGKEREG |
| | VSCISWSGDTTYYADSVKGRFTVSRDNAKNTLYLQMNSLKPEDTAMY |
| | YCAADQSLCSLAPPYNYAYWGQGTQVTVSS |
| 29 | QVQLVESGGGLVQAGGSLRLSCTASGFTFDNYVMGWFRQAPGKEREG |
| | VSCIGWSGGSTYYADSVKGRFTISRDNAKNTLYLQMNSLKPEDTAMY |
| | YCAADQGKCSLGSAGADDMDYWGRGTLVTVSS |
| 30 | EVQLVESGGGSVQAGGSLRLSCAAPGYTYCTYDMMWYRQALGKERE |
| | FVSAVYTDGSTLYADSVKGRFTISQDNAKNTLYLQMNSLKPEDTAMY |
| | YCKTETAVTYDKPCDFWGQGTQVTVSS |
| 31 | QVQLVESGGGLVQPGGSLRLSCAASGSIFSITAMGWYRQAPGKQRELV |
| | ATVTSGGSTKYVESVKGRFTISNDNAKNTVYLQMNSLKPEDTAVYYC |
| | WADIVARWNSDYNVYDDYWGQGTQVTVSS |
| 32 | QVKLEESGGGLVQEGGSLRLSCAASGTIYTDSTIYWYRQAPGKQRELV |
| | ASITRGGDTKYVDSVKGRFAISSDNAKNTVYLQMNSLKPEDTAVYYCN |
| | LYYRTGPSSYYRNDWGQGTQVTVSS |
| 33 | AVQLVESGGGLVQPGGSLRLSCAVSGIYYSINLMGWYRQAPGKPRELA |
| | ASITDSGITNYADSVKGRFTISRDNAKNTVHLQMNNLKPEDTAVYYCK |
| | PMGVNWGQGIQVTVSS |
| 34 | AVQLVESGGGLVQAGGSLRLSCTASGSILRTYEMGWYRQAPGSQRELV |
| | ASITSDGDTNYVDSVKGRFTISRDNGKNMVYLQMNSLKPDDTAVYYC |
| | NAVILSPSYSMRTSHWGQGTQVTVSS |
| 35 | QVQLVESGGGLVQAGGSLRLSCAASGNVFRFNIMGWYRQAPGNQREL |
| | VASIDDGGDRSYADSVEGRFTISRENGKKIMYLQMNSLKPEDTAVYYC |
| | AAGLGTYLNGRVSMATNYWGQGTQVTVSS |
| 36 | EVQLVESGGGLVQAGGSLRLSCAASGDTFSFAAMGWYRQAPGKQREL |
| | VASISFSGGESTVYANSVRGRFTISGDNAKNRVSLEMNNLKPEDTAVY |
| | YCTAKRGPYLPGPEYWGQGTQVTVSS |

TABLE 5
The sequences of the anti-CLL-1 V_{H}H

| SEQ ID NO | Sequence (CDRs are underlined) |
|---|---|
| 37 | QVQLVESGGDLVRPGGSLRLSCAASGFTFSIYDMNWVRQAPGKGLEW |
| | VAGISGNGYSTSYAESVKGRFTISRDNAKNTVYLQLSSLKFEDTAMYY |
| | CVRDAERWDENDLRRKGQGTQVTVSS |
| 38 | EVQLVESGGGSVQAGGSLRLSCAASGVTYSSACMGWFRQAPGKGREV |
| | VAVLYAGGSTTHYASSVKERFTISQDNAKNTVYLQMNSLKPEDTAVY |
| | YCAAALGDRSSCEWRYWGQGTQVTVSS |
| 39 | QVQLVESGGGLVQPGGSLRLSCAASGFTFSVYDMNWFRQAPGKGLEW |
| | VSGITGNGYTTSYADSVKGRFTISRDNAKNTLYLQLNSLKSEDTAMYY |
| | CAKETNRGQGTQVTVSS |
| 40 | QVQLAESGGGLVQPGGSLRLSCVASGFTFSSYDMSWVRQAPGKGVEW |
| | VSTINSGGGSTYYAESAKGRFTISRDNAKNTLYLQLNSLKTEDTAMYY |
| | CVKGFPDDDGPGELSREYNYWGQGTQVTVSS |
| 41 | EVQLVESGGALVQPGGSLRLSCTASGFLFRVYDMNWVRQAPGKGVEW |
| | IVGITNNGYTTAYADSVKGRFTISRDNTENTLFLQMNSLKPEDTAMYY |
| | CQTDNGRVRGQGTQVTVSS |
| 42 | QVQLVESGGGSVQAGGALSLSCAASGYTVRIDYMGWYRQTPGKGREP |
| | VATIASNGGTAYADSVEGRFTISQDNAKNSVYLQMNTLKPGDTAMYY |
| | CAAGTWPTLTYFGQGTQVTVSS |
| 43 | QVQLAESGGGLVQTGGSLRLSCTASGLNFGLYAMGWFRQAPGKEREG |
| | VSCINGGGGITVYSDFVKSRFTISRDNAKNTLYLQMNSLKPDDTATYYC |
| | AADRSPFGSCSSDWSRSSDWSRMAEKFGYWGQGTQVTVSS |
| 44 | QVQLVESGGGSVQAGGSLRLSCVVSAATNCRYIAWYRQAPGKAREFV |
| | STLGSDGNTNYADSVKGRFTISQGNIKNMAYLEMNSLKPEDTGMYYC |
| | GTRCQIGDDWRSSDWAQGTQVTVSS |
| 45 | QVHLVESGGGSVQSGGSLRLSCAASGYAYRSYCMGWFRQAPGKVLEG |
| | VAAIESDGTTTYADSVMGRFTISQDNAKNALYLQMNSLKPEDTAMYH |
| | CAAVKGSCDSASSDTPSYWGQGTQVTVSS |
| 46 | EVQLVESGGDLVRPGGSLRLSCAASGFTFSIYDMNWVRQAPGKGLEW |
| | VAGISGNGYSTSYAESVKGRFTISKDNAKNTVYLQLSSLKFEDTAMYY |
| | CVRGGEKWDENDLRRKGQGTQVTVSS |
| 47 | QVRLVESGGGSVQSGGSLRLSCAASGYARSSTCLGWFRQAPGKEVEGV |
| | AIIGRDGSTGYADSVKGRFTISQDNAKNTLYLHMDSLKPEDTAMYYCA |
| | AVEGGCEVSEGTGEQQLAYWGQGTQVTVSS |
| 48 | QVHLMESGGGLVQPGESLRLSCAASGFIFANYEMSWVRQAPGKVLEW |
| | VSGINSRGNATYYADSVKGRFTISRDNAEHTLYLQMNSLKPEDTAMYH |
| | CVVGGMTTDQGSPDFYWGQGTQVTVSS |
| 49 | QVKLVESGGGLVQPGGSLRLSCVASGFAFSSADMSWVRQAPGKGVEA |
| | VSVINRDGASTYYADSVKGRFTISRDNAKSTLYLQMNSLKPEDTAMYH |
| | CAVVPENEYESGSYNYWGQGTQVTVSS |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A system for inducing activity of an immune cell, comprising:
(a) a chimeric antigen receptor (CAR) comprising a first antigen binding domain which exhibits specific binding to a first epitope, a transmembrane domain, and an intracellular signaling domain devoid of a signaling domain of CD3 zeta; and
(b) a modified T cell receptor (TCR) complex comprising a second antigen binding domain that exhibits specific binding to a second epitope, wherein said second antigen binding domain is linked to:
(i) at least one TCR chain selected from an alpha chain, a beta chain, a gamma chain, and a delta chain of a T cell receptor,
(ii) an epsilon chain, a delta chain, and/or a gamma chain of cluster of differentiation 3 (CD3), or
(iii) a CD3 zeta chain.

2. The system of claim 1, wherein binding of the first antigen binding domain to the first epitope, and/or binding of the second antigen binding domain to the second epitope activates an immune cell activity of an immune cell expressing the system.

3. The system of claim 1, wherein two or more antigen binding domains are linked to, optionally in tandem, (i) at least one TCR chain selected from an alpha chain, a beta chain, a gamma chain, and a delta chain of a T cell receptor, (ii) an epsilon chain, a delta chain, and/or a gamma chain of cluster of differentiation 3 (CD3), (iii) a CD3 zeta chain, and wherein binding of the two or more antigen binding domains to their respective epitopes activates an immune cell activity of an immune cell expressing the system.

4. (canceled)

5. The system of claim 1, wherein
(i) the first epitope and the second epitope are the same;
(ii) the first epitope and the second epitope are different;
(iii) said first epitope and said second epitope are present on different antigens;
(iv) said first epitope and said second epitope are present on a common antigen;
(v) said first epitope and/or said second epitope is present on a universal antigen;
(vi) said first epitope and/or said second epitope is present on a neoantigen;
(vii) said first epitope and/or said second epitope is a neoepitope; or
(viii) said first epitope and/or said second epitope are present on one or more cell surface antigens.

6. (canceled)

7. The system of claim 1, wherein
(i) the first antigen binding domain and the second antigen binding domain comprise the same amino acid sequence;
(ii) the first antigen binding domain and the second antigen binding domain comprise different amino acid sequences;
(iii) the second antigen binding domain comprises a heterologous sequence exhibiting binding to the second epitope;
(iv) said first antigen binding domain and/or said second antigen binding domain comprises a receptor or a ligand for a receptor; or
(v) said first antigen binding domain and/or said second antigen binding domain comprises a Fab, a Fab′, a F(ab′)₂, an Fv, a single-chain Fv (scFv), minibody, a diabody, a single-domain antibody, a light chain variable domain (VL), or a variable domain (V_{H}H) of camelid antibody.

8. (canceled)

9. (canceled)

10. The system of claim 1, wherein said modified TCR comprises a third antigen binding domain linked to:
(i) said second antigen binding domain,
(ii) at least one TCR chain selected from the alpha chain, the beta chain, the gamma chain, and the delta chain of a T cell receptor,
(iii) the epsilon chain, the delta chain, and/or the gamma chain of cluster of differentiation 3 (CD3), or
(iv) the CD3 zeta chain.

11. The system of claim 1, wherein said intracellular signaling domain of said CAR is devoid of an immunoreceptor tyrosine-based activation motif (ITAM).

12. The system of claim 1, wherein said CAR further comprises a co-stimulatory domain.

13.-24. (canceled)

25. The system of claim 1, wherein said first epitope and/or said second epitope is present on a tumor-associated antigen.

26. The system of claim 25, wherein the tumor-associated antigen is selected from the group consisting of: 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, BCMA, b-Catenin, bcr-abl, bcr-abl p190 (e1a2), bcr-abl p210 (b2a2), bcr-abl p210 (b3a2), BING-4, CA-125, CAG-3, CAIX, CAMEL, Caspase-8, CD171, CD19, CD20, CD22, CD23, CD24, CD30, CD33, CD38, CD44v7/8, CD70, CD123, CD133, CDC27, CDK-4, CEA, CLCA2, CLL-1, CTAG1B, Cyp-B, DAM-10, DAM-6, DEK-CAN, DLL3, EGFR, EGFRvIII, EGP-2, EGP-40, ELF2, Ep-CAM, EphA2, EphA3, erb-B2, erb-B3, erb-B4, ES-ESO-1a, ETV6/AML, FAP, FBP, fetal acetylcholine receptor, FGF-5, FN, FR-α, G250, GAGE-1, GAGE-2, GAGE-3, GAGE-4, GAGE-5, GAGE-6, GAGE-7B, GAGE-8, GD2, GD3, GnT-V, Gp100, gp75, GPC3, GPC-2, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, L1-CAM, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, and κ-light chain.

27. The system of claim 1, wherein
(i) said first epitope and/or said second epitope is present on an immune checkpoint receptor or immune checkpoint receptor ligand;
(ii) said first epitope and/or said second epitope is present on a cytokine or a cytokine receptor; or
(iii) said first epitope and/or said second epitope is present on an antigen presented by a major histocompatibility complex (MHC).

28.-33. (canceled)

34. An isolated host cell comprising the system of claim 1.

35.-65. (canceled)

66. A method of inducing activity of an immune cell, comprising:
(a) expressing a system of claim 1 in an immune cell; and
(b) contacting a target cell with the immune cell under conditions that induce said activity of the immune cell and/or the target cell.

67.-79. (canceled)

80. A composition comprising one or more polynucleotides that encodes:
(a) a chimeric antigen receptor (CAR) comprising a first antigen binding domain having binding specificity for a first epitope, a transmembrane domain, and an intracellular signaling domain that is devoid of signaling domain of CD3 zeta; and
(b) a modified T cell receptor (TCR) complex comprising a second antigen binding domain which exhibits specific binding to a second epitope, wherein said second antigen binding domain is linked to:
(i) at least one TCR chain selected from an alpha chain, a beta chain, a gamma chain, and a delta chain of a T cell receptor,
(ii) an epsilon chain, a delta chain, and/or a gamma chain of cluster of differentiation 3 (CD3), or
(iii) a CD3 zeta chain.

81.-82. (canceled)

83. A method of producing a modified immune cell, comprising:
genetically modifying the immune cell by expressing the composition of claim 80 in said immune cell, thereby producing said modified immune cell.

84. A method of treating a cancer of a subject, said subject comprising a target cell expressing one or more antigens, the method comprising:
(a) administering to the subject an antigen-specific immune cell comprising a system of claim 1, wherein the expressed one or more antigens are recognized by the first and/or second antigen binding domain, and
(b) contacting the target cell with the antigen-specific immune cell via the first and/or second antigen binding domains under conditions that induces an immune cell activity of the immune cell against the target cell, thereby inducing death of the target cell of the cancer.

85. (canceled)

86. The method of claim 84, wherein said cancer is selected from: bladder cancer, bone cancer, brain cancer, breast cancer, cervical cancer, colon cancer, esophageal cancer, gastric cancer, glioma, head and neck cancer, kidney cancer, leukemia, acute myeloid leukemia (AML), multiple myeloma, liver cancer, lung cancer, lymphoma, melanoma, mesothelioma, medulloblastoma, ovarian cancer, pancreatic cancer, prostate cancer, rectal cancer, skin cancer, testicular cancer, tracheal cancer, and vulvar cancer.

87. The system of claim 1, wherein the system comprises:
(1) CD3ε leading peptide-anti-CLL-1 sdAb-(G4S)3-CD3ε-T2A-CD8 leading peptide-anti-CD33 sdAb-CD8 hinge-CD8 transmembrane-CD27 costimulatory domain;
(2) CD3γ leading peptide-anti-CLL-1 sdAb-(G4S)3-CD3γ-T2A-CD8 leading peptide-anti-CD33 sdAb-CD8 hinge-CD8 transmembrane-CD27 costimulatory domain;
(3) CD3ε leading peptide-anti-CLL-1 sdAb-(G4S)3-anti-CLL-1 sdAb-(G4S)3-CD3ε-T2A-CD8 leading peptide-anti-CD33 sdAb-CD8 hinge-CD8 transmembrane-CD27 costimulatory domain;
(4) CD3γ leading peptide-anti-CLL-1 sdAb-(G4S)3-anti-CLL-1 sdAb-(G4S)3-CD3γ-T2A-CD8 leading peptide-anti-CD33 sdAb-CD8 hinge-CD8 transmembrane-CD27 costimulatory domain;
(5) CD3ε leading peptide-anti-CLL-1 sdAb-(G4S)3-CD3ε-T2A-CD8 leading peptide-anti-CD33 sdAb-(G4S)3-anti-CD33 sdAb-CD8 hinge-CD8 transmembrane-CD27 costimulatory domain;
(6) CD3γ leading peptide-anti-CLL-1 sdAb-(G4S)3-CD3γ-T2A-CD8 leading peptide-anti-CD33 sdAb-(G4S)3-anti-CD33 sdAb-CD8 hinge-CD8 transmembrane-CD27 costimulatory domain;
(7) CD3δ leading peptide-anti-CLL-1 sdAb-(G4S)3-anti-CLL-1 sdAb-(G4S)3-CD3δ-T2A-CD8 leading peptide-anti-CD33 sdAb-CD8 hinge-CD8 transmembrane-CD27 costimulatory domain;
(8) CD3ε leading peptide-anti-CLL-1 sdAb-(G4S)3-CD3ε-T2A-CD3δ leading peptide-anti-CLL-1 sdAb-(G4S)3-CD3δ-T2A-CD8 leading peptide-anti-CD33 sdAb-CD8 hinge-CD8 transmembrane-CD27 costimulatory domain;
(9) CD3ε leading peptide-anti-CLL-1 sdAb-(G4S)3-CD3ε-T2A-CD3γ leading peptide-anti-CLL-1 sdAb-(G4S)3-CD3γ-T2A-CD8 leading peptide-anti-CD33 sdAb-CD8 hinge-CD8 transmembrane-CD27 costimulatory domain;
(10) CD3γ leading peptide-anti-CLL-1 sdAb-(G4S)3-CD3γ-T2A-CD3δ leading peptide-anti-CLL-1 sdAb-(G4S)3-CD3δ-T2A-CD8 leading peptide-anti-CD33 sdAb-CD8 hinge-CD8 transmembrane-CD27 costimulatory domain;
(11) CD3δ leading peptide-anti-CLL-1 sdAb-(G4S)3-CD3δ-T2A-CD8 leading peptide-anti-CD33 sdAb-(G4S)3-anti-CD33 sdAb-CD8 hinge-CD8 transmembrane-CD27 costimulatory domain; or
(12) CD3δ leading peptide-anti-CLL-1 sdAb-(G4S)3-CD3δ-T2A-CD8 leading peptide-anti-CD33 sdAb-CD8 hinge-CD8 transmembrane-CD27 costimulatory domain.

88. The system of claim 1, wherein the first antigen binding domain and the second antigen binding domain comprises an amino acid sequence selected from the group consisting of SEQ ID NOs: 23, 41, 7-22, 24-40 and 42-49.

89. The host cell of claim 34, wherein the cell is a T cell or a natural killer (NK) cell.
